(19) United States
(12) Patent Application Publication
(10) Pub. No.: US 2015/0119327 A1
(43) Pub. Date: Apr. 30, 2015

(54) DRUG SCREENING PLATFORM FOR RETT SYNDROME

(71) Applicant: THE REGENTS OF THE UNIVERSITY OF CALIFORNIA, Oakland, CA (US)

(72) Inventors: Alysson Renato Muotri, La Jolla, CA (US); Cassiano Carromeu, La Jolla, CA (US); Allan Acab, La Jolla, CA (US)

(21) Appl. No.: 14/396,697

(22) PCT Filed: Apr. 25, 2013
(86) PCT No.: PCT/US13/38260

§ 371 Date: Oct. 23, 2014

Related U.S. Application Data
(60) Provisional application No. 61/638,043, filed on Apr. 25, 2012.

(51) Int. Cl. C12Q 1/68; G01N 33/68; G01N 33/50; A61K 38/30; A61K 48/00

(52) U.S. Cl. CPC C12Q 1/6883; A61K 38/30; A61K 48/00; G01N 33/5058; G01N 33/6896; C12Q 2600/156; G01N 2800/2814; G01N 2800/50
USPC 514/8.6; 514/44 A; 435/29; 435/6.13; 506/9; 506/10; 435/6.12; 435/7.21; 435/7.1; 435/6.11

(57) ABSTRACT

The invention provides a method for restoring a neural cell having a deficiency or alteration in glutamatergic pathway affecting neuron and/or glial function comprising contacting the cell with a NMDA receptor antagonist(s) and/or modulator(s) of a glutamatergic pathway, thereby restoring the neural cell having a deficiency or alteration in glutamatergic pathways affecting neuron and/or glial function.

This invention was made with government support under Grant No. 1 DP2 OD006495-01 awarded by NIH. The government has certain rights in the invention.

Throughout this application various publications are referenced. The disclosures of these publications in their entireties are hereby incorporated by reference into this application in order to more fully describe the state of the art to which this invention pertains.

### BACKGROUND OF THE INVENTION

The study of autism spectrum disorder (ASD) risk variants is critical for the understanding of autism pathophysiology. Induced pluripotent stem cells (iPSCs) provide a valuable strategy to study the effects of these variants in living patient cells. While models have been developed for monogenic forms of ASD, no models of idiopathic ASD using iPSCs have heretofore been reported.

Rett syndrome (RTT) is a devastating disease that affects 1 in every 10,000 children born in the United States, primarily females. RTT patients undergo apparently normal development until 6-18 months of age, followed by impaired motor function, stagnation and then regression of developmental skills, hypotonia, seizures and a spectrum of autistic behaviors². Rett syndrome is a rare disease, that share routes with major developmental disorders such as autism and schizophrenia, increasing the potential impact. There is no cure for Rett syndrome and the animal model does not entirely recapitulate the human disease. Thus, having the possibility to screen drugs directly in human neurons is a major milestone.

The invention herein includes methods and compositions that involve the discovery that TRPC6 expression is regulated by MeCP2, and that mutations in TRPC6 cause Rett syndrome, revealing common pathways among ASDs. Additionally, the methods and compositions of the invention herein take into account the discovery that glutamate signaling is impaired in RTT and in a subset of idiopathic autistic patients that carry genetic alterations in this pathway.

### SUMMARY OF THE INVENTION

The invention provides a method for restoring a neural cell having a deficiency or alteration in glutamatergic pathway affecting neuron and/or glial function comprising contacting the cell with a NMDA receptor antagonist(s) and/or modulator(s) of a glutamatergic pathway, thereby restoring the neural cell having a deficiency or alteration in glutamatergic pathways affecting neuron and/or glial function.

The invention also provides a method for correcting a deficiency or alteration in glutamatergic pathway of a neural cell affecting neuron and/or glial function comprising contacting the cell with a NMDA receptor antagonist(s) and/or modulator(s) of a glutamatergic pathway, thereby correcting the deficiency or alteration in glutamatergic pathways affecting neuron and/or glial function.

The invention also provides a method for inhibiting a neurological disease or disorder in a subject having a deficiency or alteration in a glutamatergic pathway affecting neuron and/or glial function comprising administering an effective amount of a NMDA receptor antagonist(s) and/or modulator(s) of a glutamatergic pathway to the subject, thereby inhibiting the disease or disorder.

The invention also provides methods for screening candidate drugs that inhibit a neurological disease or disorder associated with a MeCP2 mutation, haploid insufficiency or a X-linked gene mutation or aberrant activity. The method comprises inducing iPSC from a male subject to undergo neuronal differentiation. The method further comprises contacting or exposing the neuronal differentiated iPSC-derived cells or neurons with candidate drugs. Further, the method comprises analyzing the eye PSC-derived cells or neurons treated with candidate drugs for an increase in neuronal networks, dendritic spine density synapses, Soma size, neuronal excitation, or calcium signaling. A decrease may be indicative of inhibiting the neurological disease when compared to mock treated cells or when compared to treated differentiated iPSC-derived cells or neurons from a wildtype or unaffected male subject.

In another embodiment, the method comprises inducing iPSC from a male subject to undergo glial cell differentiation; contacting the glial differentiated iPSC-derived cells or astrocytes with candidate drugs; and analyzing the treated cells for a decrease in calcium signaling or calcium wave propagation upon mechanical stimulation of a cell or normalizing cytokine gene expression. A decrease may be indicative of inhibiting the neurological disease when compared to mock treated cells or when compared to treated differentiated iPSC-derived cells or astrocytes from a wild-type or unaffected male subject.

The invention also provides methods for screening candidate drugs that inhibit a neurological disease or disorder associated with a TRPC6 mutation, haploid insufficiency or a X-linked gene mutation or aberrant activity. The method comprises inducing iPSC from a subject to undergo neuronal differentiation. The method further comprises contacting or exposing the neuronal differentiated iPSC-derived cells or neurons with candidate drugs. Further, the method comprises analyzing the eye PSC-derived cells or neurons treated with candidate drugs for an increase in neuronal networks, dendritic spine density synapses, Soma size, neuronal excitation, or calcium signaling. A decrease may be indicative of inhibiting the neurological disease when compared to mock treated cells or when compared to treated differentiated iPSC-derived cells or neurons from a wildtype or unaffected male subject.

In an additional embodiment, the method comprises inducing iPSC from a subject to undergo glial cell differentiation; contacting the glial differentiated iPSC-derived cells or astrocytes with candidate drugs; and analyzing the treated cells for an increase in calcium signaling or calcium wave propagation upon mechanical stimulation of a cell or normalizing cytokine gene expression. An increase being indicative of inhibiting the neurological disease when compared to mock treated cells or when compared to treated differentiated iPSC-derived cells or astrocytes from a wild-type or unaffected male subject.

The invention further provides a method for correcting a defect associated with X-chromosome gene mutation in glial cells affecting neuronal network formation or function in a subject, such method comprising transplantation of a population of glial cells enriched with an active X-chromosome expressing non-mutant allele to the subject, thereby correcting a defect associated with X-chromosome gene mutation in glial cells affecting neuronal network formation or function in a subject.

The invention also provides a method for restoring PSD-95 expression levels in a subject with altered PSD-95 expression, wherein the subject is treated with an effective amount of any of Acetazolamide (also referred as N-(5-sulfamoyl-1,3,4-thiadiazol-2-yl)acetamide), a carbonic anhydrase inhibitor), BIX-01294 (having the formula C₂₈H₃₈N₆O₂.3HCl (CAS No. 1392399-03-9, 935693-62-2 (free base)), Zonisamide (also referred as benzo[d]isoxazol-3-ylmethanesulfonamide), Forskolin (also referred as (3R,4aR,5S,6S,6aS,10S,10aR,10bS)-6,10,10b-trihydroxy-3,4-a,7,7,10a-pentamethyl-1-oxo-3-vinyldodecahydro-1H-benzo chromen-5-yl acetate), Tubastatin A (also referred as N-Hydroxy-4-(2-methyl-1,2,3,4-tetrahydro-pyrido[4,3-b]indol-5-ylmethyl)-benzamide), 7,8 Dihydroxyflavone, Topiramate (also referred as 2,3:4,5-Bis-O-(1-methylethylidene)-beta-D-fructopyranose sulfamate), AR-A014418 (also referred as N-[(4-Methoxyphenyl)methyl]-N′-(5-nitro-2-thiazolyl)urea), Amitriptyline (also referred as 3-(10,11-dihydro-5H-dibenzo[a,d]cycloheptene-5-ylidene)-N,N-dimethylpropan-1-amine), LM22A-3, Aripiprazole (also referred as 7-{4-[4-(2,3-Dichlorophenyl)piperazin-1-yl]butoxy}-3,4-dihydroquinolin-2(1H)-one), Hyperzine A or huperzine A (also referred as (1R,9S,13E)-1-Amino-13-ethylidene-11-methyl-6-azatricyclo[7.3.1.0^{2,7}]trideca-2(7),3,10-trien-5-one), MK-677 (also referred to as Ibutamoren or (R)-1′-(2-methylalanyl-O-benzyl-D-seryl)-1-(methylsulfonyl)-1,2-dihydrospiro[indole-3,4′-piperidine]), Chlormezanone (also referred to as 2-(4-chlorophenyl)-3-methyl-1,1-dioxo-1,3-thiazinan-4-one), Cyctothiazide (also referred to as 3-(bicyclo[2.2.1]hept-5-en-2-yl)-6-chloro-3,4-dihydro-2H-1,2,4-benzothiadiazine-7-sulfonamide 1,1-dioxide), Pioglitazone (also referred to as (RS)-5-(4-[2-(5-ethylpyridin-2-yl)ethoxy]benzyl)thiazolidine-2,4-dione), Memantine (also referred to as 3,5-dimethyltricyclo[3.3.1.1^{3,7}]decan-lamine or 3,5-dimethyladamantan-1-amine), No Glutamine, DON, CBX, Valproic Acid (VPA), DAPT (also referred to as LY-374973, N—[N-(3,5-Difluorophenacetyl)-L-alanyl]-S-phenylglycine t-butyl ester), Aminoguanidine, Dizocilpine (INN) (also referred to as MK-801 or [5R,10S]-[+]-5-methyl-10,11-dihydro-5H-dibenzo[a,d]cyclohepten-5,10-imine), Curcumin (also referred to as (1E,6E)-1,7-Bis(4-hydroxy-3-methoxyphenyl)-1,6-heptadiene-3,5-dione), Resveratrol (3,5,4′-trihydroxy-trans-stilbene), Ceftriaxone (also referred to as (6R,7R)-7-{[(2Z)-2-(2-amino-1,3-thiazol-4-yl)->2-(methoxyimino)acetyl]amino}-3-[(2-methyl-5,6-dioxo-1,2,5,6-tetrahydro-1,2,4-triazin-3-yl)thio]methyl-8-oxo-5-thia-1-azabicyclo[4.2.0]oct-2-ene-2-carboxylic acid), Epigallocatechin (also referred to as Gallocatechol or gallocatechin), Gingerol (also referred to as (S)-5-hydroxy-1-(4-hydroxy-3-methoxyphenyl)-3-decanone), Gly-Pro-Glu, and/or Insulin-like growth factor 1 (IGF-1) (also referred to as somatomedin C).

The invention also provides a method for identifying subjects with an increased risk for developing a disease or disorder selected from a group consisting of Rett Syndrome (RTT), idiopathic autism, severe neonatal encephalopathy, schizophrenia, X-linked mental retardation, deficiency in glutamatergic pathways of the glial cells, neuronal networks with a deficiency in glutamatergic pathways affecting the formation of excitatory synapses, and/or a subset of neurological disorders with a deficiency in glutamatergic pathways affecting the formation of excitatory synapses comprising detecting in cells from the subject a mutation in a TRPC6 gene participating in a glutamatergic pathway in neuronal or glial cells, the presence of the mutation in the TRPC6 gene being indicative of an increased risk for the disease or disorder.

The invention also provides a method for diagnosing a subject with an increased risk for developing Rett Syndrome (RTT), idiopathic autism, severe neonatal encephalopathy, schizophrenia, X-linked mental retardation, deficiency in glutamatergic pathways of the glial cells, neuronal networks with a deficiency in glutamatergic pathways affecting the formation of excitatory synapses, and/or a subset of neurological disorders with a deficiency in glutamatergic pathways affecting the formation of excitatory synapses, comprising detecting in the cells from the subject a mutation in a TRPC6 gene and having decreased neuronal gene expression affecting one or more pathways comprising neurotrophin signaling pathway, IGF signaling pathway, pathway with synaptic protein, NeuN gene pathway, and glutamate-glutamine transport pathway.

The invention also provides a method for inhibiting idiopathic autism associated with a deficiency or alteration in glutamatergic pathways affecting neuron and/or glial function or TRPC6 haploinsufficiency or TRPC6 gene mutation comprising administering an effective amount of hyperforin and/or flufenamic acid (FFA) or equivalents thereof to a subject, thereby treating, inhibiting, or preventing the development of idiopathic autism associated with a deficiency or alteration in glutamatergic pathways affecting neuron and/or glial function or TRPC6 haploinsufficiency or TRPC6 gene mutation.

The invention also provides a method for reversing TRPC6 haplo-insufficiency leading to altered expression of TRPC6-responsive gene(s) comprising administering an effective amount of hyperforin and/or flufenamic acid (FFA) or equivalents thereof to a subject, thereby reversing TRPC6 haploinsufficiency and normalizing expression of TRPC6-responsive gene(s).

The invention also provides a method for diagnosing or identifying a subject with an increased risk of developing a neurological disease or disorder. The method comprises inducing iPSC from a subject to undergo neuronal or glial cell differentiation; and analyzing the neuronal or glial cells for one or more of the following: synaptic deficiency, reduced dendritic spine density, reduced glutamatergic synapses, decreased neurite soma size, reduced neurite length, reduced number of glutamate vesicles, reduced number of VGLUT1 puncta or cluster along MAP2-positive processes of neurons, reduced dendritic complexity measured as a function of number of crossings for each distance from the cell body, decreased neuronal nuclei size, reduced neuronal nuclei sphericity, reduced neuronal spike frequency, decreased transient Ca²⁺ concentration, reduced repetitive intracellular Ca²⁺ concentration, decreased amplitude of Ca²⁺ oscillation, reduced Na⁺ current density, decreased action potential, reduced action potential burst trains, reduced firing rate of neurons in whole cell patch clamp recording, reduced number of synapsin puncta or cluster along MAP2-positive processes of neurons, reduced PSD-95 expression level, reduced neuronal networks, reduced astrocyte networks, reduced calcium signaling, reduced calcium wave propagation to surrounding cells upon mechanical stimulation of an individual cell, or disregulated gene expression in the neuronal or glial cell compared to neuronal differentiated iPSC-derived cells or neurons or glial differentiated iPSC-derived cells or astrocytes from an unaffected subject. The presence of one or more of the above being indicative of an increased risk of developing a neurological disease or disorder thereby, identifying a subject with an increased risk of developing a neurological disease or disorder.

### BRIEF DESCRIPTION OF THE FIGURES

FIG. 1. Mapping the breakpoints in the patient with the 46, XY, t(3;11)(p21;q22) karyotype. (A) Allele frequency distribution plot for chromosomes 3 and 11 generated by SNP array genotyping showing no gain or loss of genetic material on these chromosomes. (B) Schematic view of the BAC probes used and the surrounding breakpoint area on chromosome 3. RP11 probes marked in red span the breakpoint, while the black ones do not. The shared region between probes RP11-780O20 and RP11-109N8 narrow the breakpoint area to a region inside VPRBP gene. Blue arrows indicate open reading frames. (C) FISH imaging showing that RP11-780O20 probe (green signal) binds to normal and derivative chromosome 3 and to derivative chromosome 11, indicating that the probe spans the breakpoint (arrowheads). (D) Schematic view of BAC probes used and surrounding areas on chromosome 11. Shared region between probes RP11-153K15 and RP11-141E21 places the breakpoint into TRPC6. (E) FISH image showing BAC probe RP11-153K15 (arrowheads) bound to normal chromosome 11 and both derivatives chromosomes 3 and 11.

FIG. 2. TRPC6 channels regulate expression of neuronal development genes. (A) Decreasing expression of candidate genes upon TRPC6 stimulation with hyperforin/FFA. (B) Genes that were up-regulated in the TRPC6-mut genetic background after hyperforin/FFA treatment. (C) CREB phosphorylation increase after 15 and 30 minutes of hyperforin stimulation normalized to non-stimulated cells. The levels of CREB phosphorylation reached in DPSC from TRPC6-mut patient after TRPC6 activation with hyperforin is significantly lower compared to control sample (n=3, p<0.05).

FIG. 3. Derivation of NPCs and neurons from iPSCs. (A) Representative images depicting morphological changes during neuronal differentiation. Bar=100 μm. (B) NPCs are positive for neural precursor markers Musashi-1 and Nestin. Bar=50 μm. (C) Representative images of cells after neuronal differentiation. iPSC-derived neurons express mature neuronal markers such as GABA, MAP2 and Synapsin. (D) Examples of distinct cortical neuronal subtypes present differentiating cultures after 3 weeks. Bar=30 μm. (E) The percentage of neurons obtained with this protocol is ˜30%, measured by MAP2 staining and infection with the syn::EGFP lentiviral vector. Most of the MAP2-positive cells expressed VGLUT1 in contrast with 12% of neurons expressing GABA. Ctip2-positive neurons were more abundant (˜15%) whereas Tbr1-positive neurons were present in a small percentage in the population (˜5%) at the end of the differentiation protocol. (F) Morphology of neurons patched for electrophysiological recording. (G) Representative recordings of evoked action potentials in iPSC-derived neurons in response to current steps under current patch clamps. (H) A representative Na⁺ and K⁺ currents in iPSC-derived neurons.

FIG. 4. Alterations in TRPC6-mutant patient's neural cells. (A) Ca²⁺ influx dynamics through TRPC6 channels activated by hyperforin plus FFA are reduced in patient's cells. Oscillations generated by hyperforin and FFA treatment were normalized to the fluorescence of the resting level (F₀), synchronized and averaged. (B) The average peak of Ca²⁺ influx in the approximately 100 cells analyzed is reduced by about 40% in the patient's NPCs compared to the control sample when cells were stimulated by hyperforin and FFA (p<0.001). (C) NPCs cell cycle analysis indicates that there is no significant difference between patient and control regarding the percentage of cells in G1, S and G2 phases of cell cycle (n=3 for each clone analyzed). (D) Representative images of TRPC6-mutant and control neurons before and after neurite tracing. Neuronal morphology was visualized using the syn::EGFP lentiviral vector. Bar graphs show that the number of vertices and the total length of TRPC6-mutant neurites are reduced compared to control and another idiopathic ASD patient. Bar=50 μm. (E) Representative images of neuronal spines in control and TRPC6-mutant neurons. Bar graphs show that spine density in TRPC6-mutant neurons is reduced compared to controls. Specific shRNA against TRPC6 (shTRPC6) was used to confirm that the phenotype was caused by loss of TRPC6 function. (F) Representative images of neurons stained for VGLUT1 and MAP2. Bar graphs show that the number of glutamate vesicles in TRPC6-mutant neurons is significantly reduced compared to controls. Data shown as mean±s.e.m. (G) Control neurons expressing the shRNA against TRPC6 showed reduced numbers of VGLUT1 puncta when compared to neurons expressing a scrambled shRNA (control). Bar=5 μm. Data shown as mean±s.e.m. (H) Whole cell Na⁺ current of TRPC6-mutant neurons is significantly less than that of control. (I) Na⁺ current density of TRPC6-mutant neurons is also significantly less than that of control. (J) TRPC6 protein levels are reduced in neurons derived from an RTT iPSC clone expressing a non-functional version of MeCP2. Numbers of neurons analyzed (n) are shown within the bars in graphs D-I. For all iPSC clones used refer to Table S3.

FIG. 5. TRPC6 regulates neural development of adult-born neurons in the dentate gyrus of hippocampus. (A) Sample confocal images of neurons expressing shRNA-control and shRNA-TRPC6-1 at 14 dpi (days post retroviral injection), Green: GFP and blue: DAPI. Scale bar 20 μm. Also shown are divided areas of dentate gyrus. 1: inner granule cell layer; 2: middle granule cell layer; 3: upper granule cell layer; 4: molecular layer. (B) Summary of cell body localization of GFP⁺ newborn neurons under different experimental conditions. Values represent mean±SEM (n=3; *: p<0.01; ANOVA). (C) Three-dimensional confocal reconstruction of dendritic trees of GFP⁺ dentate granule cells expressing shRNA-control or shRNA-TRPC6-1 at 14 dpi. Scale bar: 20 μm. (D) Sholl analysis of dendritic complexity of GFP⁺ neurons at 14 dpi. Values represent mean±SEM (n=3; *: p<0.05; ANOVA). (E) A sample DIC image of a newborn neuron patched in whole-cell configuration in an acute slice of the hippocampus (Top panel). (F) Firing rate of repetitive action potentials of GFP⁺ neurons under current clamp in response to a 1 s current injection steps at 21 dpi. Shown in left is a sample trace of a GFP⁺ neuron expressing shRNA-control and in right expressing shRNA-TRPC6#-1. (G) Summary of the mean firing rate of newborn neurons. Values represent mean±SD (n=3; *: p<0.01; ANOVA).

FIG. 6. Confirmation of TRPC6 disruption. (A) qPCR data showing the levels of expression of TRPC6 exons 6, 12 and 13 relative to exon 4 for the patient, parents and mean of 6 controls. The patient is the only individual that has a decrease of ˜50% on the levels of expression of exons 12 and 13. (B) Genotyping of rs12366144 SNP in TRPC6 exon 6 (left) and rs12805398 SNP in exon 13 (right). The control sample maintains heterozygosis for both SNPs at the transcriptional level (arrows). On the other hand, the patient does not present one of the alleles in exon 13 when the cDNA is sequenced, indicating that TRPC6 is transcribed until the breakpoint on the disrupted chromosome. (C) Examples of microsatellite genotyping for parenthood confirmation.

FIG. 7. Generation and characterization of iPSCs. (A) Cells emerging from the dental pulp. (B) Established dental pulp stem cells lineage. (C) iPSC colony emerging from the co-culture system with mEFs. (D) Isolated iPSC colony. (E) iPSC colony stained for pluripotency markers SOX2 and Lin28. (F) iPSC colony stained for pluripotency markers TRA-1-81 and Nanog. Bar=100 μm. (G) Karyotypes of TRPC6mut iPSCs and WT iPSCs showing the stable karyotype of these cells after more than 20 passages. Arrows point to the de novo translocation between chromosomes 3 and 11. (H) Hematoxin-eosin stained slices of the teratomas obtained after iPSC injection in nude mice. The presence of tissues containing the three different embryonic layers indicates that the DPSCs were fully reprogrammed to a pluripotent state. Bar=250 μm. (I) Pearson's correlation coefficients of microarray profiles of triplicate WT DPSC, TRPC6mut DPSC, WT-iPSC clone 1, TRPC6-mut iPSC clones 4 and 6 and the hESC line HUES6. Color bar indicates the level of correlation (from 0 to 1), with color bar reporting log 2 normalized expression values (red/blue indicates high/low relative expression). (J) Hierarchical cluster obtained from expression microarray data: the iPSCs lineages obtained clustered with hESCs, indicating that these cells have a similar expression profile, while DPSCs lineages clustered all together in a separate group. Three different clones from the patient were used for microarray analysis and all the samples were run in triplicate.

FIG. 8. Electrophysiological recordings and morphological phenotypes of iPSC-derived cortical neurons. (A) Representative recordings of Na⁺ current from an iPSC-derived neuron was blocked by 10 μM Tetrodotoxin (TTX). (B) K⁺ current was blocked using 20 mM tetraethalammonium (TEA). (C) Bar graphs show that spine density in TRPC6-mut neurons (black bars) is reduced compared to controls. (D) Bar graphs show that the number of glutamate vesicles (measured by VGLUT1 puncta along MAP2-positive processes) in TRPC6-mut neurons (black bars) is significantly reduced compared to controls. WT controls and the human ES cell line HUES6 data (white bars) were re-plotted from Marchetto et al, 2010 (Marchetto et al., 2010). Data shown as mean±s.e.m.

FIG. 9. In vivo validation of TRPC6 knockdown. (A) Validation of the efficacy of shRNAs against TRPC6 in vitro. Retroviral constructs expressing different shRNAs were co-transfected with an expression construct for TRPC6 into HEK-293 cells. Lysates were subjected to Western blot analysis for TRPC6 and GAPDH (a sample image is shown on top). Also shown is the quantification of knockdown efficacy. The densitometry measurement was first normalized to that of GAPDH and then to that of shRNA-control expression sample. Values represent mean±SD (n=3; *: p<0.01; ANOVA). (B) Sample confocal images of neurons expressing shRNA-control and shRNA-TRPC6-1 at 28 dpi. Green: GFP and blue: DAPI. Scale bar 50 μm. Also shown are sample images of three-dimensional confocal reconstruction of dendritic trees of GFP⁺ dentate granule cells at 28 dpi. Scale bar: 50 μm. (C) Summary of cell body localization of GFP⁺ newborn neurons under different experimental conditions at 28 dpi. Values represent mean±SEM (n=3; *: p<0.01; ANOVA). (D) Sholl analysis of dendritic complexity of GFP⁺ neurons at 28 dpi. Values represent mean±SD (n=3). (E) Western analysis of TRPC6 protein level under different conditions. Retroviral constructs expressing different shRNAs were co-transfected with an expression construct for an shRNA-resistant form of TRPC6-WT (TRPC6-WT^{R}) into HEK-293 cells. Lysates were subjected to Western blot analysis for TRPC6 and GAPDH. (F) Rescue of cell migration phenotype by expression of TRPC6-WT^{R} at 14 dpi. Retroviruses co-expressing GFP and TrpC6-WT^{R} were co-injected with retroviruses co-expressing dsRed and shRNA-TRPC6-1 into adult mouse dentate gyri. The cell body localization of GFP⁺, DsRed⁺ and GFP⁺ DsRed⁺ neurons are quantified. Values represent mean±SD (n=3; *: p<0.01; ANOVA). (G) Behavioral analysis on Trpc6 KO and HET mice. Mean of body weight, defecation and urination episodes during the test, showing that wild type (WT), heterozygote (HET) and knockout (KO) Trpc6 animals are not physiologically different in these regards. Evaluation of time spent in freezing behavior and in grooming behavior showed no significant different between the groups. Social interaction was assessed through the evaluation of time spent with novel object or strange animal and nose-to-nose contact. Adult mice (6-8 weeks old, male) in a C57BL/6 background were used for the study. At least 12 animals per group were utilized in biological replicates. Experimenter was blind to the genotypes. The data were analyzed using the non-parametric ANOVA test Kruskal-Wallis. All procedures followed the institutional guidelines.

FIG. 10. (A) Principal component analysis. Screen plot of the first 200 components identifies the first three as contributing the greatest amount of variation. (B) Population stratification control plots. The three largest principal components of genotypes for SSC cases (green) and NINDS controls (blue) were plotted against one another (PC=principal component, EV=Eigenvalue). (C) Removal of ancestral outliers. The interquartile range (IQR) distance around the median of the study population cluster was calculated. A threshold that included all the NINDS controls was determined to lie at 6 IQRs from the third quartile, and SSC cases beyond this threshold were excluded as ancestral outliers. Included samples are in blue, and excluded samples (outliers) are in green.

FIG. 11. (Top) A schematic diagram showing that fibroblasts from patients are reprogrammed to a pluripotent state and further differentiated into neuro progenitor cells (NPCs). These NPCs can be expanded in appropriated culture conditions and, under the right signals, induced to differentiated into postmitotic neurons. (Bottom) A 96-well plate of an in-cell western assay. An Odyssey machine was used to estimate the number of synapses by detecting specific wavelengths in a 96- or 24-well plates.

FIG. 12. A table identifying genes that are downregulated in the absence of MeCP2. These genes are located downstream of MeCP2 and validated by qPCR (tables) or by protein levels. This data shows that many pathways are affected, including the glutamate pathway, responsible for the formation of excitatory synapses in the brain.

FIG. 13. A table identifying genes that are downregulated in the absence of MeCP2. These genes are located downstream of MeCP2 and validated by qPCR (tables) or by protein levels. This data shows that many pathways are affected, including the glutamate pathway, responsible for the formation of excitatory synapses in the brain.

FIG. 14. Bar graphs showing two readouts for a drug screening platform. The first two graphs show the quantification of protein levels using infrared detection (in cell western) in neurons derived from a control (WT83), a RTT patient (Q83X) and a patient with MePC2 duplication (2M). The graph on the bottom shows the PSD95 levels after treatment with therapeutic agents listed in FIG. 15. The horizontal bar is the control level. Drugs that increase the amount of PSD95 in RTT neurons were those that reached control level.

FIG. 15. A table identifying therapeutic agents from FIG. 14. The intensity of the PSD95 level is discriminated by no effect (0), mild (+) or strong (++).

FIG. 16. Validation of the drug screening by Western blot, showing that positive therapeutic agents increase both PSD95 and synapsin protein levels in RTT neurons.

FIG. 17. Graphs of a multielectrode array (MEA) designated MED64. The field activity of 64 channels was measured in a neuronal network derived from a non-affected individual (control) and patient with Rett syndrome (RTT) treated with or without Memantine (Mem). The bar graph compares the number of synchronized burst activities recorded in a 5 minute time frame.

FIG. 18. A readout showing a single channel from the MEA and comparing the conditions of wildtype cells, RTT cells untreated with memantine and RTT cells treated with memantine.

FIG. 19. A bar graph showing gene expression measured by quantitative PCR showed differences between astrocytes derived from controls (WT) and RTT patients. Rett astrocytes show an increased expression of the main astrocyte-related genes, GFAP, S100b, Aquaporin4 and vimentin which is indicative of a hyper-reactive astrocyte. BMP2, BMP4 and GDNF are downregulated in Rett astrocytes thereby indicating a possible defective synthesis of those molecules.

FIG. 20. Photograph of astrocytes. Astrocytes are known to propagate a calcium wave when mechanically stimulated (circle) indicating cell communication. Stimulating a set of control WT astrocytes mechanically by the use of the Fluo4AM calcium dye shows live spread of this calcium wave over time.

FIG. 21. A line graph and a photograph showing that Rett astrocytes did not propagate a calcium wave.

FIG. 22. A diagram showing the affect of astrocytes on RTT neurons.

FIG. 23. A photograph showing a neuron stained in green and astrocytes stained in red in a layer under the neuron.

FIG. 24. A photograph showing the effects of co-culturing WT neurons, healthy control astrocytes, RTT neurons, and RTT astrocytes.

FIG. 25. Morphometric neuronal quantifications were performed with the use of the neurolucida software. A significant rescue was achieved related to the number of dendrites in RTT neurons, with numbers similar to that of the controls.

FIG. 26. Graphs and tables showing cytokine expression of RTT and WT astrocytes. IL-8 and IL-10 showed the most dramatic differences between RTT and control WT astrocytes among the 40 differentially expressed cytokines.

FIG. 27. Top: Gene expression of iPSC-derived astrocytes compared to primary astrocytes. Bottom: Calcium waves induced by mechanical stimulation of single cell in a monolayer of iPSC-derived astrocytes.

FIG. 28. Presence of A2B5 progenitor cells in early passages of iPSC-derived astrocytes. NG2-positive cells migrating out of neurospheres. FACS analyses of iPSC-derived astrocytes using CD44 and GFAP antibodies.

FIG. 29. Photographs showing early stages of astrocyte differentiation from iPSC. Most of the cells are GFAP-positive and a few are S100B positive.

FIG. 30. Photographs of iPSC-derived astrocytes which have been passaged thereby resulting in a homogenous population of GFAP/S100B positive cells.

FIG. 31. A photograph of astrocytes derived from H9 cells.

### DETAILED DESCRIPTION OF THE INVENTION

Unless defined otherwise, all technical and scientific terms used herein have the same meaning as is commonly understood by one of ordinary skill in the art to which this invention belongs. All patents, applications, published applications and other publications referred to herein are incorporated by reference in their entirety.

The phrase “pharmaceutically acceptable carrier” refers to any carrier known to those skilled in the art to be suitable for the particular mode of administration.

### I. Methods of the Invention

The invention provides a method for restoring a neural cell having a deficiency or alteration in glutamatergic pathway affecting neuron and/or glial function (e.g. malfunction) comprising contacting the cell with a NMDA receptor antagonist(s) and/or modulator(s) of a glutamatergic pathway, thereby restoring the neural cell having a deficiency or alteration in glutamatergic pathways affecting neuron and/or glial function. Restoring a neural cell having a deficiency includes any one or more of restoring synaptic deficiency, restoring neurite soma size, restoring neurite length, restoring the number of glutamate vesicles, restoring VGLUT1 puncta or cluster along MAP2-positive processes of neurons, restoring dendritic complexity measured as a function of number of crossings for each distance from the cell body, restoring neuronal nuclei size, restoring neuronal nuclei sphericity, restoring neuronal spike frequency, restoring Na⁺ current density, restoring action potential, restoring synapsin puncta or cluster along MAP2-positive processes of neurons, restoring PSD-95 expression level, restoring neuronal networks, restoring astrocyte networks, restoring calcium signaling, restoring calcium wave propagation to surrounding cells upon mechanical stimulation of an individual cell, and/or normalizes gene expression in the neuronal or glial cell. Restoring a neural cell may be partial or complete.

The invention also provides a method for correcting a deficiency or alteration in glutamatergic pathway of a neural cell affecting neuron and/or glial function (e.g. malfunction) comprising contacting the cell with a NMDA receptor antagonist(s) and/or modulator(s) of a glutamatergic pathway, thereby correcting the deficiency or alteration in glutamatergic pathways affecting neuron and/or glial function. Correcting a deficiency or alteration in glutamatergic pathway may be partial or complete.

The invention also provides methods for inhibiting a neurological disease or disorder in a subject having a deficiency or alteration in a glutamatergic pathway neuron and/or glial function. The subject may include, but is not limited to, human, monkey, pig, horse, cow, dog and cat.

The method comprises administering an effective amount of a NMDA receptor antagonist to the subject thereby inhibiting the disease or disorder. Additionally, in one embodiment of the invention, the subject may be further administered with a modulator of a glutamatergic pathway. In the embodiment where more than one agent is administered, administration may be made concurrently or sequentially. Further, the agents may be administered together or separately.

In an alternative embodiment of the invention, the method comprises administering an effective amount of a modulator of a glutamatergic pathway to the subject thereby inhibiting the disease or disorder.

The progression of the disease or disorder may be inhibited by any of the methods of the invention. In another embodiment, the neurological disease or disorder may be treated by any of the methods of the invention. In yet a further embodiment, the neurological disease or disorder may be prevented by any of the methods of the invention.

In one embodiment of the invention, the deficiency or alteration in a glutamatergic pathway is associated with TRPC6 mutation or haploid insufficiency. Further, in another embodiment the deficiency or alteration in a glutamatergic pathway is associated with a TRPC6 mutation or haploid insufficiency and a MeCP2 mutation or haploid insufficiency. TRPC6 is a human transient receptor potential cation channel, subfamily C, member 6, for example as exemplified in NCBI Gene ID: 7225, the sequence for which is incorporated by reference herein.

Examples of neurological disease or disorders include, but are not limited to, Rett syndrome (RTT), idiopathic autism, severe neonatal encephalopathy, schizophrenia, autism spectrum disorder (ASD) and X-linked mental retardation.

In accordance with the practice of the invention, inhibiting the neurological disease or disorder may include any one, two, three, four, five or more of (1) restoring synaptic deficiency, (2) increasing dendritic spine density, (3) increasing glutamatergic synapses, (4) restoring neurite soma size, (5) restoring neurite length, (6) restoring the number of glutamate vesicles, (7) restoring VGLUT1 puncta or cluster along MAP2-positive processes of neurons, (8) restoring dendritic complexity measured as a function of number of crossings for each distance from the cell body, (9) restoring neuronal nuclei size, restores neuronal nuclei sphericity, (10) restoring neuronal spike frequency, (11) increasing transient Ca²⁺ concentration, (12) increasing repetitive intracellular Ca²⁺ concentration, (13) increasing amplitude of Ca²⁺ oscillation, (14) restores Na⁺ current density, (15) restores action potential, (16) increases action potential burst trains, (17) restoring firing rate of neurons in whole cell patch clamp recording, (18) restoring synapsin puncta or cluster along MAP2-positive processes of neurons, (19) restoring PSD-95 expression level, (20) restoring neuronal networks, (21) restoring astrocyte networks, (22) restoring calcium signaling, (23) restoring calcium wave propagation to surrounding cells upon mechanical stimulation of an individual cell, or (24) normalizing gene expression in the neuronal or glial cell within the central nervous system or peripheral nervous system of a subject.

Examples of NMDA receptor antagonist(s) include, but are not limited to, 3,5-Dimethyl-tricyclo[3.3.1.13,7]decan-1-amine hydrochloride (Memantine hydrochloride), 1-Aminocyclobutane-1-carboxylic acid (ACBC), D-(−)-2-Amino-5-phosphonopentanoic acid (D-AP5), L-(+)-2-Amino-5-phosphonopentanoic acid (L-AP5), D-(−)-2-Amino-7-phosphonoheptanoic acid (D-AP7), N,N′-1,4-Butanediylbisguanidine sulfate (arcaine sulfate), (R)-4-Carboxyphenylglycine ((R)-4CPG), (S)-4-Carboxyphenylglycine ((S)-4CPG), (E)-(±)-2-Amino-4-methyl-5-phosphono-3-pentenoic acid (CGP 37849), (E)-(±)-2-Amino-4-methyl-5-phosphono-3-pentenoic acid ethyl ester (CGP 39551), [(1S)-1-[[(7-Bromo-1,2,3,4-tetrahydro-2,3-dioxo-5-quinoxalinyl)methyl]amino]ethyl]phosphonic acid hydrochloride (CGP 78608 hydrochloride), cis-4-[Phosphomethyl]-piperidine-2-carboxylic acid (CGS 19755), 7-Chloro-4-hydroxyquinoline-2-carboxylic acid (7-Chlorokynurenic acid), (2R,3S)-β-p-Chlorophenylglutamic acid ((2R,3S)-Chlorpheg), 6-Cyano-7-nitroquinoxaline-2,3-dione (CNQX), 1-[2-(4-Hydroxyphenoxy)ethyl]-4-[(4-methylphenyl)methyl]-4-piperidinol hydrochloride (Co 101244 hydrochloride; PD 174494; or Ro 63-1908), GEXXVAKMAAXLARXNIAKGCKVNCYP (Conantokin-R), GEXXYQKMLXNLRXAEVKKNA (Conantokin-T), 3-((R)-2-Carboxypiperazin-4-yl)-propyl-1-phosphonic acid ((R)-CPP), (RS)-3-(2-Carboxypiperazin-4-yl)-propyl-1-phosphonic acid ((RS)-CPP), D-4-[(2E)-3-Phosphono-2-propenyl]-2-piperazinecarboxylic acid (D-CPP-ene; Midafotel; or SDZ EAA 494), (9α,13α,14α)-3-Methoxy-17-methylmorphinan hydrobromide (Dextromethorphan hydrobromide), 5,7-Dichloro-4-hydroxyquinoline-2-carboxylic acid (5,7-Dichlorokynurenic acid), (±)-1-(1,2-Diphenylethyl)piperidine maleate, α-(4-Chlorophenyl)-4-[(4-fluorophenyl)methyl]-1-piperidineethanol (Eliprodil), 2-Phenyl-1,3-propanedioldicarbamate (Felbamate), N-[2-Amino-6-[[4-fluorophenyl)methyl]amino]-3-pyridinyl]carbamic acid ethyl ester maleate (Flupirtine maleate), 4,6-Dichloro-3-[(1E)-3-oxo-3-(phenylamino)-1-propenyl]-1H-indole-2-carboxylic acid sodium salt (Gavestinel; GV 150526A), (S)-(−)-3-Amino-1-hydroxypyrrolidin-2-one ((S)-(−)-HA-966), (R)-(+)-3-Amino-1-hydroxypyrrolidin-2-one ((R)-(+)-HA-966), (6aS,10aS)-3-(1,1-Dimethylheptyl)-6a,7,10,10a-tetrahydro-1-hydroxy-6,6-dimethyl-6H-dibenzo[b,d]pyran-9-methanol (HU 211; or Dexanabinol), N—(N-(4-Hydroxyphenylacetyl)-3-aminopropyl)-(N′-3-aminopropyl)-1,4-butanediamine (N-(4-Hydroxyphenylacetyl)spermine), (1R*,2S*)-erythro-2-(4-Benzylpiperidino)-1-(4-hydroxyphenyl)-1-propanol hemitartrate (Ifenprodil hemitartrate), (1S*,2S*)-threo-2-(4-Benzylpiperidino)-1-(4-hydroxyphenyl)-1-propanol hemitartrate (threo Ifenprodil hemitartrate), 2-(2-Chlorophenyl)-2-(methylamino)cyclohexanone hydrochloride (Ketamine hydrochloride), (S)-(+)-2-(2-Chlorophenyl)-2-(methylamino)cyclohexanone hydrochloride ((S)-(+)-Ketamine hydrochloride), trans-2-Carboxy-5,7-dichloro-4-phenylaminocarbonylamino-1,2,3,4-tetrahydroquinoline (L-689,560), 7-Chloro-3-(cyclopropylcarbonyl)-4-hydroxy-2(1H)-quinolinone (L-701,252), 7-Chloro-4-hydroxy-3-(3-phenoxy)phenyl-2(1H)-quinolinone (L-701,324), 4-(4-Chlorophenyl)-4-hydroxy-N,N-dimethyl-α,α-diphenyl-1-piperidinebutanamide hydrochloride (Loperamide hydrochloride), (2R*,4S*)-4-(1H-Tetrazol-5-ylmethyl)-2-piperidinecarboxylic acid (LY 233053), [3S-(3α,4aα,6β,8aα)]-Decahydro-6-(phosphonomethyl)-3-isoquinolinecarboxylic acid (LY 235959), (5R,10S)-(+5-Methyl-10,11-dihydro-5H-dibenzo[a,d]cylcohepten-5,10-imine maleate ((−)-MK 801 maleate), (5S,10R)-(+)-5-Methyl-10,11-dihydro-5H-dibenzo[a,d]cyclohepten-5,10-imine maleate ((+)-MK 801 maleate; MK-801; or Dizocilpine), 2-Amino-2-(2-chlorophenyl)cyclohexanone hydrochloride (Norketamine hydrochloride), D-[[1-(2-Nitrophenyl)ethyl]carbamoyl]-2-amino]-5-phosphonopentanoic acid (NPEC-caged-D-AP5), 4,4′-(Pentamethylenedioxy)dibenzamidine bis-2-hydroxyethanesulfonate salt (Pentamidine isethionate), 1-(1-Phenylcyclohexyl)piperidine hydrochloride (Phencyclidine hydrochloride), 4-(Phosphonomethyl)-2-piperazinecarboxylic acid (PMPA), (2S*,3R*)-1-(Phenanthren-2-carbonyl)piperazine-2,3-dicarboxylic acid (PPDA), (2R*,4S*)-4-(3-Phosphonopropyl)-2-piperidinecarboxylic acid (PPPA; or LY 257883), 2-Amino-N-(1-methyl-1,2-diphenylethyl)acetamide hydrochloride (Remacemide hydrochloride; or FPL 12924AA), 1-[2-(4-Chlorophenyl)ethyl]-1,2,3,4-tetrahydro-6-methoxy-2-methyl-7-isoquinolinol hydrochloride (Ro 04-5595 hydrochloride), (αR,βS)-α-(4-Hydroxyphenyl)-β-methyl-4-(phenylmethyl)-1-piperidinepropanol maleate (Ro 25-6981 maleate), 3,4-Dimethoxy-N-[4-(3-nitrophenyl)-2-thiazolyl]benzenesulfonamide (Ro 61-8048), 4-[3-[4-(4-Fluorophenyl)-1,2,3,6-tetrahydro-1(2H)-pyridinyl]-2-hydroxypropoxy]benzamide hydrochloride (Ro 8-4304 hydrochloride), (S)-α-Amino-2′,4′-dichloro-4-hydroxy-5-(phosphonomethyl)-[1,1′-biphenyl]-3-propanoic acid (SDZ 220-040), (S)-α-Amino-2′-chloro-5-(phosphonomethyl) [1,1′-biphenyl]-3-propanoic acid (SDZ 220-581), N,N′-1,10-Decanediylbisguanidine sulfate (Synthalin sulfate), 3-Chloro-4-fluoro-N-[4-[[2-(phenylcarbonyl)hydrazino]carbonyl]benzyl]benzenesulfonamide (TCN 201), N-(Cyclohexylmethyl)-2-[(5-[(phenylmethyl)amino]-1,3,4-thiadiazol-2-yl thio]acetamide (TCN 213), 1,3-Dihydro-5-[3-[4-(phenylmethyl)-1-2H-benzimidazol-2-one (TCS 46b), nitrous oxide (N₂O), Dextrorphan, Selfotel, Amantadine, Dextrallorphan, Eticyclidine, Gacyclidine, Ibogaine, Ethanol, Methoxetamine, Rolicyclidine, Tenocyclidine, Methoxydine (4-meo-pcp), Tiletamine, Xenon, Neramexane, Etoxadrol, Dexoxadrol, NEFA, Delucemine, 8A-PDHQ, Aptiganel (Cerestat; or CNS-1102), HU-211, Rhynchophylline, 1-Aminocyclopropanecarboxylic acid, 7-Chlorokynurenate, Kynurenic acid, and Lacosamide, and/or derivatives thereof, including any acid and/or salt forms thereof.

Further, the administration of NMDA receptor antagonist(s) may restore one or more neuronal phenotypes, such as restore synaptic deficiency, increase dendritic spine density, increase glutamatergic synapses, restore neurite soma size, restore neurite length, restore number of glutamate vesicles, restore VGLUT1 puncta or cluster along MAP2-positive processes of neurons, restore dendritic complexity measured as a function of number of crossings for each distance from the cell body, restore neuronal nuclei size, restore neuronal nuclei sphericity, restore neuronal spike frequency, increase transient Ca²⁺ concentration, increase repetitive intracellular Ca²⁺ concentration, increase amplitude of Ca²⁺ oscillation, restore Na⁺ current density, restore action potential, increases action potential burst trains, restore firing rate of neurons in whole cell patch clamp recording, restores synapsin puncta or cluster along MAP2-positive processes of neurons, restore PSD-95 expression level, restore neuronal networks, restore astrocyte networks, restore calcium signaling, restore calcium wave propagation to surrounding cells upon mechanical stimulation of an individual cell, or normalize gene expression in the neuronal or glial cell within the central nervous system or peripheral nervous system of a subject.

Examples of modulators of a glutamatergic pathway include, but are not limited to, TRPC6 modulators and MeCP2 modulators. These modulators may modulate the phosphorylation state of a CREB transcription factor so as to normalized neuronal or glial gene expression.

Further examples of modulators of a glutamatergic pathway include, but are not limited to, Acetazolamide (also referred to as N-(5-sulfamoyl-1,3,4-thiadiazol-2-yl)acetamide), a carbonic anhydrase inhibitor), BIX-01294 (having the formula C₂₈H₃₈N₆O₂.3HCl (CAS No. 1392399-03-9, 935693-62-2 (free base)), a G9a histone methyltransferease (G9aHMTase) inhibitor, Zonisamide (also referred to as benzo[d]isoxazol-3-ylmethanesulfonamide), a sulfonamide anticonvulsant, Forskolin (also referred to as (3R,4aR,5S,6S,6aS,10S,10aR,10bS)-6,10,10b-trihydroxy-3,4-a,7,7,10a-pentamethyl-1-oxo-3-vinyldodecahydro-1H-benzo[f]chromen-5-yl acetate), a labdane diterpene, an adenylyl cyclase activator, Tubastatin A (also referred to as N-Hydroxy-4-(2-methyl-1,2,3,4-tetrahydro-pyrido[4,3-b]indol-5-ylmethyl)-benzamide), 7,8 Dihydroxyflavone, Topiramate (also referred to as 2,3:4,5-Bis-O-(1-methylethylidene)-beta-D-fructopyranose sulfamate), a histone deacetylase HDAC6 inhibitor, AR-A014418 (also referred to as N-[(4-Methoxyphenyl)methyl]-N-(5-nitro-2-thiazolyl)urea), a glycogen synthase kinase 3 (GSK3) inhibitor, Amitriptyline (also referred to as 3-(10,11-dihydro-5H-dibenzo[a,d]cycloheptene-5-ylidene)-N,N-dimethylpropan-1-amine), a serotonin-norepinephrine reuptake inhibitor, a 5-HT_{2A}, 5-HT_{2C}, 5-HT₃, 5-HT₆, 5-HT₇, α1-adrenergic, H₁, H₂, H₄, and mACh receptor antagonist, σ1 receptor agonist, a sodium, calcium, and potassium channel blocker, a TrkA and TrkB receptor agonist, LM22A-3, a BDNF mimic, NBI-31772, an insulin-like growth factor 1 (IGF-1) potentiator, ING-135, an ampakine, an α-amino-3-hydroxy-5-methyl-4-isoxazolepropionic acid (AMPA) receptor potentiator, CX-546, LM22A-4, Aripiprazole (also referred to as 7-{4-[4-(2,3-Dichlorophenyl)piperazin-1-yl]butoxy}-3,4-dihydroquinolin-2(1H)-one), a dopamine and serotonin receptor modulator, Hyperzine A or huperzine A (also referred to as (1R,9S,13E)-1-Amino-13-ethylidene-11-methyl-6-azatricyclo[7.3.1.0^{2,7}]trideca-2 (7),3,10-trien-5-one), an acetylcholinesterase and NMDA inhibitor, MK-677 (also referred to as Ibutamoren or (R)-1′-(2-methylalanyl-O-benzyl-D-seryl)-1-(methylsulfonyl)-1,2-dihydrospiro[indole-3,4′-piperidine]), a growth hormone secretagogue, Chlormezanone (also referred to as 2-(4-chlorophenyl)-3-methyl-1,1-dioxo-1,3-thiazinan-4-one), a GABA receptor potentiator, Cyctothiazide (also referred to as 3-(bicyclo[2.2.1]hept-5-en-2-yl)-6-chloro-3,4-dihydro-2H-1,2,4-benzothiadiazine-7-sulfonamide 1,1-dioxide), a positive modulator of AMPA receptor, Pioglitazone (also referred to as (RS)-5-(4-[2-(5-ethylpyridin-2-yl)ethoxy]benzyl)thiazolidine-2,4-dione), an agonist of the peroxisome proliferator activated receptor gamma (PPARγ), Memantine (also referred to as 3,5-dimethyltricyclo[3.3.1.1^{3,7}]decan-1 amine or 3,5-dimethyladamantan-1-amine), an NMDA receptor antagonist, a glutamate antagonist, a medium, plasma or extracellular fluid deficient in glutamine or glutamate, a glutamate antagonist, DON (also referred to as 6-Diazo-5-oxo-L-norleucine or (Z,5 S)-5-Amino-1-diazonio-6-hydroxy-6-oxohex-1-en-2-olate; chemical formula C6H9N3O3 (CAS No. 157-03-9), a glutamine antagonist, CBX (also referred to as carbenoxolone, (3β)-3-[(3-carboxypropanoyl)oxy]-11-oxoolean-12-en-30-oic acid, or (2S,4aS,6aS,6bR,8aR,10S,12aS,12bR,14bR)-10-(3-carboxypropanoyloxy)-2,4-a,6a,6b,9,9,12a-heptamethyl-13-oxo-1,2,3,4,4a,5,6,6a,6b,7,8,8a,9,10,11,12,12a,12b,13,14b-icosahydropicene-2-carboxylic acid); chemical formula C₃₄H₅₀O₇ (CAS No. 5697-56-3)), a gap junction blocker, Valproic Acid (VPA), a histone deacetylase (HDAC) inhibitor, DAPT (also referred to as LY-374973, N—[N-(3,5-Difluorophenacetyl)-L-alanyl]-S-phenylglycine t-butyl ester), a gamma-secretase inhibitor, Aminoguanidine, an iNOS inhibitor, Dizocilpine (INN) (also referred to as MK-801 or [5R,10S]-[+]-5-methyl-10,11-dihydro-5H-dibenzo[a,d]cyclohepten-5,10-imine), a NMDA receptor antagonist, Curcumin (also referred to as (1E,6E)-1,7-Bis(4-hydroxy-3-methoxyphenyl)-1,6-heptadiene-3,5-dione), Resveratrol (3,5,4′-trihydroxy-trans-stilbene), a curcuminoid, a natural phenol, an anti-inflammatory agent, Ceftriaxone (also referred to as (6R,7R)-7-{[(2Z)-2-(2-amino-1,3-thiazol-4-yl)->2-(methoxyimino)acetyl]amino}-3-{[(2-methyl-5,6-dioxo-1,2,5,6-tetrahydro-1,2,4-triazin-3-yl)thio]methyl}-8-oxo-5-thia-1-azabicyclo[4.2.0]oct-2-ene-2-carboxylic acid), Epigallocatechin (also referred to as Gallocatechol or gallocatechin), Gingerol (also referred to as (S)-5-hydroxy-1-(4-hydroxy-3-methoxyphenyl)-3-decanone), Gly-Pro-Glu, and/or Insulin-like growth factor 1 (IGF-1) (also referred to as somatomedin C).

The chemical structure of LM22A-3 is as follows:

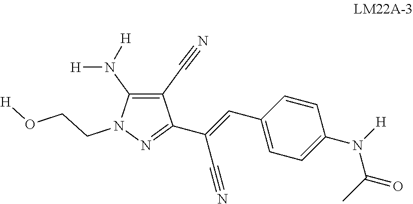 [SMILES: CC(=O)Nc1ccc(/C=C(\C#N)c2nn(CCO)c(N)c2C#N)cc1]

In one embodiment, the method further comprises administering an effective amount of a cytokine(s) and/or modulator(s) of cytokine activity to the subject.

In one embodiment, the administration of cytokine(s) and/or modulator(s) of cytokine activity may result in one or more of the following: restores synaptic deficiency, increases dendritic spine density, increases glutamatergic synapses, restores neurite soma size, restores neurite length, restores number of glutamate vesicles, restores VGLUT1 puncta or cluster along MAP2-positive processes of neurons, restores dendritic complexity measured as a function of number of crossings for each distance from the cell body, restores neuronal nuclei size, restores neuronal nuclei sphericity, restores neuronal spike frequency, increases transient Ca²⁺ concentration, increases repetitive intracellular Ca²⁺ concentration, increases amplitude of Ca²⁺ oscillation, restores Na⁺ current density, restores action potential, increases action potential burst trains, restores firing rate of neurons in whole cell patch clamp recording, restores synapsin puncta or cluster along MAP2-positive processes of neurons, restores PSD-95 expression level, restores neuronal networks, restores astrocyte networks, restores calcium signaling, restores calcium wave propagation to surrounding cells upon mechanical stimulation of an individual cell, or normalizes gene expression in the neuronal or glial cell within the central nervous system or peripheral nervous system of a subject or a combination thereof.

Examples of cytokine include, but are not limited to, Bone morphogenetic protein (BMP) 2 (BMP2), BMP3, BMP4, CD70, interleukin (IL) 10 (IL-10), IL-17B, IL-18, Bone morphogenetic protein 5 (BMPS), CD40 Ligand (also referred to as gp39 or CD40L), colony stimulating factor 2 (CSF2 or Granulocyte macrophage colony-stimulating factor 2 (also referred to as GM-CSF or sargramostim)), CSF-3 (also referred to as G-CSF and filgrastim)), interferon A4 (IFNA4), interleukin 13 (IL-13), IL-15, IL-23A, IL-3, IL-4, IL-5, Inhibin, beta A, (INHBA), Leukemia inhibitory factor (LIF), tumor growth factor beta-1 (TGF-β1), tumor growth factor beta-2 (TGF-β2), tumor growth factor beta-3 (TGF-B3), Tumor necrosis factor superfamily 12 (TNF-SF12), Tumor necrosis factor superfamily 13 (TNF-SF13B), Tumor necrosis factor superfamily 8 (TNF-SF8), and/or Alpha-taxilin (TXLNA).

In one embodiment, the modulator(s) of cytokine activity may be an agent which decreases production, decreases secretion, decreases half-life, decreases activity or neutralizes activity of any one or more of Bone morphogenetic protein 5 (BMPS), CD40 Ligand (also referred to as gp39 or CD40L), colony stimulating factor 2 (CSF2 or Granulocyte macrophage colony-stimulating factor 2 (also referred to as GM-CSF or sargramostim)), CSF-3 (also referred to as G-CSF and filgrastim)), interferon A4 (IFNA4), interleukin 13 (IL-13), IL-15, IL-23A, IL-3, IL-4, IL-5, Inhibin, beta A, (INHBA), Leukemia inhibitory factor (LIF), tumor growth factor beta-1 (TGF-B1), tumor growth factor beta-2 (TGF-β2), tumor growth factor beta-3 (TGF-β3), Tumor necrosis factor superfamily 12 (TNF-SF12), Tumor necrosis factor superfamily 13 (TNF-SF13B), Tumor necrosis factor superfamily 8 (TNF-SF8), and/or Alpha-taxilin (TXLNA).

In another embodiment, the modulator(s) of cytokine activity may be an agent which increases production, increases secretion, increases half-life or enhances activity of any one or more of BMP2, BMP3, BMP4, CD70, IL-10, IL-17B, or IL-18.

In accordance with the invention, the agent may be a nucleic acid, protein or synthetic chemical compound and/or derivatives thereof.

In one embodiment, the nucleic acid comprises a gene therapy vector and a coding sequence or part of a coding sequence for any one or more of Bone morphogenetic protein 5 (BMP5), CD40 Ligand (also referred to as gp39 or CD40L), colony stimulating factor 2 (CSF2 or Granulocyte macrophage colony-stimulating factor 2 (also referred to as GM-CSF or sargramostim)), CSF-3 (also referred to as G-CSF and filgrastim)), interferon A4 (IFNA4), interleukin 13 (IL-13), IL-15, IL-23A, IL-3, IL-4, IL-5, Inhibin, beta A, (INHBA), Leukemia inhibitory factor (LIF), tumor growth factor beta-1 (TGF-β1), tumor growth factor beta-2 (TGF-β2), tumor growth factor beta-3 (TGF-β3), Tumor necrosis factor superfamily 12 (TNF-SF12), Tumor necrosis factor superfamily 13 (TNF-SF13B), Tumor necrosis factor superfamily 8 (TNF-SF8), Alpha-taxilin (TXLNA), BMP2, BMP3, BMP4, CD70, IL-10, IL-17B, and/or IL-18.

As used herein, a coding sequence includes a nucleic sequence derived or corresponding to a protein coding region of a gene. For example, a coding sequence may be in the form of a cDNA or mRNA. The cDNA or mRNA may be purified or isolated. Furthermore, a coding sequence may be used to design siRNA, shRNA, anti-sense RNA, anti-sequence oligonucleotides or other forms of regulatory nucleic acid or macromolecule that recognizes the coding sequence and modulate its expression.

In another embodiment, the nucleic acid may be a siRNA, shRNA, anti-sense RNA or anti-sense oligonucleotide directed to a coding sequence for any one or more of Bone morphogenetic protein 5 (BMPS), CD40 Ligand (also referred to as gp39 or CD40L), colony stimulating factor 2 (CSF2 or Granulocyte macrophage colony-stimulating factor 2 (also referred to as GM-CSF or sargramostim)), CSF-3 (also referred to as G-CSF and filgrastim)), interferon A4 (IFNA4), interleukin 13 (IL-13), IL-15, IL-23A, IL-3, IL-4, IL-5, Inhibin, beta A, (INHBA), Leukemia inhibitory factor (LIF), tumor growth factor beta-1 (TGF-β1), tumor growth factor beta-2 (TGF-β2), tumor growth factor beta-3 (TGF-β3), Tumor necrosis factor superfamily 12 (TNF-SF12), Tumor necrosis factor superfamily 13 (TNF-SF13B), Tumor necrosis factor superfamily 8 (TNF-SF8), and/or Alpha-taxilin (TXLNA).

In yet another embodiment, the nucleic acid comprises a gene therapy vector, and a coding sequence for MeCP2 or TRPC6 or a coding sequence for a regulator of MeCP2 or TRPC6 gene expression; or a coding sequence for Bone morphogenetic protein 5 (BMPS), CD40 Ligand (also referred to as gp39 or CD40L), colony stimulating factor 2 (CSF2 or Granulocyte macrophage colony-stimulating factor 2 (also referred to as GM-CSF or sargramostim)), CSF-3 (also referred to as G-CSF and filgrastim)), interferon A4 (IFNA4), interleukin 13 (IL-13), IL-15, IL-23A, IL-3, IL-4, IL-5, Inhibin, beta A, (INHBA), Leukemia inhibitory factor (LIF), tumor growth factor beta-1 (TGF-β1), tumor growth factor beta-2 (TGF-β2), tumor growth factor beta-3 (TGF-β3), Tumor necrosis factor superfamily 12 (TNF-SF12), Tumor necrosis factor superfamily 13 (TNF-SF13B), Tumor necrosis factor superfamily 8 (TNF-SF8), and/or Alpha-taxilin (TXLNA).

In one embodiment, the protein comprises a neutralizing antibody directed against any of Bone morphogenetic protein 5 (BMP5), CD40 Ligand (also referred to as gp39 or CD40L), colony stimulating factor 2 (CSF2 or Granulocyte macrophage colony-stimulating factor 2 (also referred to as GM-CSF or sargramostim)), CSF-3 (also referred to as G-CSF and filgrastim)), interferon A4 (IFNA4), interleukin 13 (IL-13), IL-15, IL-23A, IL-3, IL-4, IL-5, Inhibin, beta A, (INHBA), Leukemia inhibitory factor (LIF), tumor growth factor beta-1 (TGF-β1), tumor growth factor beta-2 (TGF-β2), tumor growth factor beta-3 (TGF-β3), Tumor necrosis factor superfamily 12 (TNF-SF12), Tumor necrosis factor superfamily 13 (TNF-SF13B), Tumor necrosis factor superfamily 8 (TNF-SF8), and/or Alpha-taxilin (TXLNA).

In another embodiment, the peptide or cell-penetrating peptide modulates the expression, secretion, half-life or activity of any of Bone morphogenetic protein 5 (BMP5), CD40 Ligand (also referred to as gp39 or CD40L), colony stimulating factor 2 (CSF2 or Granulocyte macrophage colony-stimulating factor 2 (also referred to as GM-CSF or sargramostim)), CSF-3 (also referred to as G-CSF and filgrastim)), interferon A4 (IFNA4), interleukin 13 (IL-13), IL-15, IL-23A, IL-3, IL-4, IL-5, Inhibin, beta A, (INHBA), Leukemia inhibitory factor (LIF), tumor growth factor beta-1 (TGF-β1), tumor growth factor beta-2 (TGF-β2), tumor growth factor beta-3 (TGF-β3), Tumor necrosis factor superfamily 12 (TNF-SF12), Tumor necrosis factor superfamily 13 (TNF-SF13B), Tumor necrosis factor superfamily 8 (TNF-SF8), and/or Alpha-taxilin (TXLNA).

In another embodiment, the antibody enhances the activity of any of Bone morphogenetic protein 5 (BMP5), CD40 Ligand (also referred to as gp39 or CD40L), colony stimulating factor 2 (CSF2 or Granulocyte macrophage colony-stimulating factor 2 (also referred to as GM-CSF or sargramostim)), CSF-3 (also referred to as G-CSF and filgrastim)), interferon A4 (IFNA4), interleukin 13 (IL-13), IL-15, IL-23A, IL-3, IL-4, IL-5, Inhibin, beta A, (INHBA), Leukemia inhibitory factor (LIF), tumor growth factor beta-1 (TGF-β1), tumor growth factor beta-2 (TGF-β2), tumor growth factor beta-3 (TGF-β3), Tumor necrosis factor superfamily 12 (TNF-SF12), Tumor necrosis factor superfamily 13 (TNF-SF13B), Tumor necrosis factor superfamily 8 (TNF-SF8), Alpha-taxilin (TXLNA), BMP2, BMP3, BMP4, CD70, IL-10, IL-17B, IL-18, MeCP2, TRPC6, a regulator of MeCP2, a regulator of TRPC6, MeCP2 gene, TRPC6 gene, a gene for a regulator of MeCP2 gene, or a gene for a regulator of TRPC6 gene.

In another embodiment, the synthetic chemical compound may be a modulator of a MeCP2 or TRPC6 gene or protein activity or components of the glutamatergic pathway in neuronal or glial cells. The modulator of TRPC6 activity may be hyperforin, flufenamic acid (FFA) and/or derivatives thereof.

The invention also provides methods for screening candidate drugs that inhibit a neurological disease or disorder associated with a MeCP2 mutation, haploid insufficiency or a X-linked gene mutation or aberrant activity. The method comprises inducing iPSC from a male subject to undergo neuronal differentiation. The method further comprises contacting or exposing the neuronal differentiated iPSC-derived cells or neurons with candidate drugs. Further, the method comprises analyzing the eye PSC-derived cells or neurons treated with candidate drugs for an increase in neuronal networks, dendritic spine density synapses, Soma size, neuronal excitation, or calcium signaling. A decrease may be indicative of inhibiting the neurological disease when compared to mock treated cells or when compared to treated differentiated iPSC-derived cells or neurons from a wildtype or unaffected male subject.

In another embodiment, the method comprises inducing iPSC from a male subject to undergo glial cell differentiation; contacting the glial differentiated iPSC-derived cells or astrocytes with candidate drugs; and analyzing the treated cells for a decrease in calcium signaling or calcium wave propagation upon mechanical stimulation of a cell or normalizing cytokine gene expression. A decrease may be indicative of inhibiting the neurological disease when compared to mock treated cells or when compared to treated differentiated iPSC-derived cells or astrocytes from a wild-type or unaffected male subject.

In one embodiment, the iPSC may exhibit reduced variability associated with dosage compensation of the X-chromosome in mammals which results in the differentiating or differentiated cell derived from an induced pluripotent stem cell (iPSC) of female origin either expressing genes from the maternal or paternal X-chromosome.

The invention also provides methods for screening candidate drugs that inhibit a neurological disease or disorder associated with a TRPC6 mutation, haploid insufficiency or a X-linked gene mutation or aberrant activity. The method comprises inducing iPSC from a subject to undergo neuronal differentiation. The method further comprises contacting or exposing the neuronal differentiated iPSC-derived cells or neurons with candidate drugs. Further, the method comprises analyzing the eye PSC-derived cells or neurons treated with candidate drugs for an increase in neuronal networks, dendritic spine density synapses, Soma size, neuronal excitation, or calcium signaling. A decrease may be indicative of inhibiting the neurological disease when compared to mock treated cells or when compared to treated differentiated iPSC-derived cells or neurons from a wildtype or unaffected male subject.

In an additional embodiment, the method comprises inducing iPSC from a subject to undergo glial cell differentiation; contacting the glial differentiated iPSC-derived cells or astrocytes with candidate drugs; and analyzing the treated cells for an increase in calcium signaling or calcium wave propagation upon mechanical stimulation of a cell or normalizing cytokine gene expression. An increase being indicative of inhibiting the neurological disease when compared to mock treated cells or when compared to treated differentiated iPSC-derived cells or astrocytes from a wild-type or unaffected male subject.

In one embodiment, the method for screening candidate drugs may be performed using an automated, high throughput screening system. For example, the automated system may estimate the number of synapses by detecting specific wavelengths. This may be done in a 24-well, 96-well or higher well density format or a multi-well format.

The invention also provides a method for correcting a defect associated with X-chromosome gene mutation in glial cells which affect neuronal network formation or function in a subject. The method comprises transplantation of a population of glial cells enriched with an active X-chromosome expressing non-mutant allele to the subject, thereby correcting a defect associated with X-chromosome gene mutation in glial cells affecting neuronal network formation or function in a subject.

In one embodiment, the population of glial cells enriched with an active X chromosome expressing a non-mutant allele may be obtained from a pluripotent stem cell or induced pluripotent stem cell of the subject.

In one embodiment of the invention, the glial cell may be an astrocyte.

The invention also provides a method for restoring PSD-95 expression levels in a subject with altered PSD-95 expression. The subject may be treated with an effective amount of any of Acetazolamide (also referred to as N-(5-sulfamoyl-1,3,4-thiadiazol-2-yl)acetamide), a carbonic anhydrase inhibitor, BIX-01294 (having the formula C₂₈H₃₈N₆O₂.3HCl (CAS No. 1392399-03-9, 935693-62-2 (free base)), a G9a histone methyltransferease (G9aHMTase) inhibitor, Zonisamide (also referred to as benzo[d]isoxazol-3-ylmethanesulfonamide), a sulfonamide anticonvulsant, Forskolin (also referred to as (3R,4aR,5S,6S,6aS,10S,10aR, 10bS)-6,10,10b-trihydroxy-3,4-a,7,7,10a-pentamethyl-1-oxo-3-vinyldodecahydro-1H-benzo[f]chromen-5-yl acetate), a labdane diterpene, an adenylyl cyclase activator, Tubastatin A (also referred to as N-Hydroxy-4-(2-methyl-1,2,3,4-tetrahydro-pyrido[4,3-b]indol-5-ylmethyl)-benzamide), 7,8 Dihydroxyflavone, Topiramate (also referred to as 2,3:4,5-Bis-O-(1-methylethylidene)-beta-D-fructopyranose sulfamate), a histone deacetylase HDAC6 inhibitor, AR-A014418 (also referred to as N-[(4-Methoxyphenyl)methyl]-N-(5-nitro-2-thiazolyl)urea), a glycogen synthase kinase 3 (GSK3) inhibitor, Amitriptyline (also referred to as 3-(10,11-dihydro-5H-dibenzo[a,d]cycloheptene-5-ylidene)-N,N-dimethylpropan-1-amine), a serotonin-norepinephrine reuptake inhibitor, a 5-HT_{2A}, 5-HT_{2C}, 5-HT₃, 5-HT₆, 5-HT₇, α1-adrenergic, H₁, H₂, H₄, and mACh receptor antagonist, σ1 receptor agonist, a sodium, calcium, and potassium channel blocker, a TrkA and TrkB receptor agonist, LM22A-3, a BDNF mimic, NBI-31772, an insulin-like growth factor 1 (IGF-1) potentiator, ING-135, an ampakine, an α-amino-3-hydroxy-5-methyl-4-isoxazolepropionic acid (AMPA) receptor potentiator, CX-546, LM22A-4, Aripiprazole (also referred to as 7-{4-[4-(2,3-Dichlorophenyl)piperazin-1-yl]butoxy}-3,4-dihydroquinolin-2(1H)-one), a dopamine and serotonin receptor modulator, Hyperzine A or huperzine A (also referred to as (1R,9S,13E)-1-Amino-13-ethylidene-11-methyl-6-azatricyclo[7.3.1.0^{2,7}]trideca-2 (7),3,10-trien-5-one), an acetylcholinesterase and NMDA inhibitor, MK-677 (also referred to as Ibutamoren or (R)-1′-(2-methylalanyl-O-benzyl-D-seryl)-1-(methylsulfonyl)-1,2-dihydrospiro[indole-3,4′-piperidine]), a growth hormone secretagogue, Chlormezanone (also referred to as 2-(4-chlorophenyl)-3-methyl-1,1-dioxo-1,3-thiazinan-4-one), a GABA receptor potentiator, Cyctothiazide (also referred to as 3-(bicyclo[2.2.1]hept-5-en-2-yl)-6-chloro-3,4-dihydro-2H-1,2,4-benzothiadiazine-7-sulfonamide 1,1-dioxide), a positive modulator of AMPA receptor, Pioglitazone (also referred to as (RS)-5-(4-[2-(5-ethylpyridin-2-yl)ethoxy]benzyl)thiazolidine-2,4-dione), an agonist of the peroxisome proliferator activated receptor gamma (PPARγ), Memantine (also referred to as 3,5-dimethyltricyclo[3.3.1.1^{3,7}]decan-1 amine or 3,5-dimethyladamantan-1-amine), an NMDA receptor antagonist, a glutamate antagonist, a medium, plasma or extracellular fluid deficient in glutamine or glutamate, a glutamate antagonist, DON (also referred to as 6-Diazo-5-oxo-L-norleucine or (Z,5 S)-5-Amino-1-diazonio-6-hydroxy-6-oxohex-1-en-2-olate; chemical formula C6H9N3O3 (CAS No. 157-03-9), a glutamine antagonist, CBX (also referred to as carbenoxolone, (3β)-3-[(3-carboxypropanoyl)oxy]-11-oxoolean-12-en-30-oic acid, or (2S,4aS,6aS,6bR,8aR,10S,12aS,12bR,14bR)-10-(3-carboxypropanoyloxy)-2,4-a,6a,6b,9,9,12a-heptamethyl-13-oxo-1,2,3,4,4a,5,6,6a,6b,7,8,8a,9,10,11,12,12a,12b,13,14b-icosahydropicene-2-carboxylic acid); chemical formula C₃₄H₅₀O₇ (CAS No. 5697-56-3)), a gap junction blocker, Valproic Acid (VPA), a histone deacetylase (HDAC) inhibitor, DAPT (also referred to as LY-374973, N—[N-(3,5-Difluorophenacetyl)-L-alanyl]-S-phenylglycine t-butyl ester), a gamma-secretase inhibitor, Aminoguanidine, an iNOS inhibitor, Dizocilpine (INN) (also referred to as MK-801 or [5R,10S]-[+]-5-methyl-10,11-dihydro-5H-dibenzo[a,d]cyclohepten-5,10-imine), a NMDA receptor antagonist, Curcumin (also referred to as (1E,6E)-1,7-Bis(4-hydroxy-3-methoxyphenyl)-1,6-heptadiene-3,5-dione), Resveratrol (3,5,4′-trihydroxy-trans-stilbene), a curcuminoid, a natural phenol, an anti-inflammatory agent, Ceftriaxone (also referred to as (6R,7R)-7-{[(2Z)-2-(2-amino-1,3-thiazol-4-yl)->2-(methoxyimino)acetyl]amino}-3-[(2-methyl-5,6-dioxo-1,2,5,6-tetrahydro-1,2,4-triazin-3-yl)thio]methyl-8-oxo-5-thia-1-azabicyclo[4.2.0]oct-2-ene-2-carboxylic acid), Epigallocatechin (also referred to as Gallocatechol or gallocatechin), Gingerol (also referred to as (5)-5-hydroxy-1-(4-hydroxy-3-methoxyphenyl)-3-decanone), Gly-Pro-Glu, or Insulin-like growth factor 1 (IGF-1) (also referred to somatomedin C). Additionally, in an embodiment of the invention, methods for inhibiting neurological disease or disorder may be effected by restoring PSD-95 expression.

The invention also provides a method for diagnosing or identifying a subject at risk for developing a neurological disease or disorder. The neurological disease or disorder includes, but are not limited to, Rett Syndrome (RTT), idiopathic autism, severe neonatal encephalopathy, schizophrenia, X-linked mental retardation, deficiency in glutamatergic pathways of the glial cells, neuronal networks with a deficiency in glutamatergic pathways affecting the formation of excitatory synapses, and/or a subset of neurological disorders with a deficiency in glutamatergic pathways affecting the formation of excitatory synapses. The method comprises detecting in cells from the subject a mutation in a TRPC6 gene participating in a glutamatergic pathway in neuronal or glial cells. The presence of the mutation in the TRPC6 gene being indicative of an increased risk for the disease or disorder.

In one embodiment, the method further comprises detecting a mutation in a MeCP2 gene. In another embodiment, the TRPC6 gene has any of the mutations, M1K, Q3X, P47A, Y207S, L353F, P439R, E466K, A560V, F795L, and K808N as set forth in Supplementary Table S4.

The invention also provides a method for diagnosing whether a subject is at risk for developing Rett Syndrome (RTT), idiopathic autism, severe neonatal encephalopathy, schizophrenia, X-linked mental retardation, deficiency in glutamatergic pathways of the glial cells, neuronal networks with a deficiency in glutamatergic pathways affecting the formation of excitatory synapses, and/or a subset of neurological disorders with a deficiency in glutamatergic pathways affecting the formation of excitatory synapses. In one embodiment, the method comprises detecting in the cells from the subject a mutation in a TRPC6 or MeCP2 gene and determining whether the cell exhibit decreased neuronal gene expression affecting one or more of the following pathways comprising neurotrophin signaling pathway, IGF signaling pathway, pathway with synaptic protein, NeuN gene pathway, and glutamate-glutamine transport pathway. In one embodiment, the decreased neuronal gene expression affecting the neurotrophin signaling pathway involves the BDNF, NGFR, or NTF4 genes. In another embodiment, the decreased neural gene expression affecting the IGF signaling pathway involves IGF 1 or IGF2 genes. In yet another embodiment, the decreased neuronal gene expression affecting the pathway with synaptic protein involves the PSD-95, VGlut1, VGlut2, or syn1 genes.

In yet a further embodiment, the decreased neural gene expression affecting glutamate-glutamine transport pathway involves EAAT2 or EAAT4 genes. Additionally, GABRA5, GNAI1, GRIA1, GRIA4, GRIN2A, GRM4, GRM5 or GRM7 genes may be involved.

The invention also provides a method for inhibiting idiopathic autism associated with a deficiency or alteration in glutamatergic pathways affecting neuron and/or glial function or TRPC6 haploinsufficiency or TRPC6 gene mutation comprising administering an effective amount of hyperforin and/or flufenamic acid (FFA) or equivalents thereof to a subject, thereby treating, inhibiting, or preventing the development of idiopathic autism associated with a deficiency or alteration in glutamatergic pathways affecting neuron and/or glial function or TRPC6 haploinsufficiency or TRPC6 gene mutation.

The invention also provides methods for reversing TRPC6 haplo-insufficiency leading to altered expression of TRPC6-responsive gene(s) comprising administering an effective amount of hyperforin and/or flufenamic acid (FFA) or equivalents thereof to a subject, thereby reversing TRPC6 haploinsufficiency and normalizing expression of TRPC6-responsive gene(s).

In one embodiment, the TRPC6-responsive gene(s) comprises SEMA3A, EPHA4, CLDN11, MAP2 or INA.

In another embodiment, the TRPC6 modulator is hyperforin or flufenamic acid (FFA).

The invention also provides a method for diagnosing or identifying whether a subject is at risk of developing a neurological disease or disorder. In an embodiment of the invention, the method comprises inducing iPSC from a subject to undergo neuronal or glial cell differentiation; and analyzing the neuronal or glial cells for one or more of the following: synaptic deficiency, reduced dendritic spine density, reduced glutamatergic synapses, decreased neurite soma size, reduced neurite length, reduced number of glutamate vesicles, reduced number of VGLUT1 puncta or cluster along MAP2-positive processes of neurons, reduced dendritic complexity measured as a function of number of crossings for each distance from the cell body, decreased neuronal nuclei size, reduced neuronal nuclei sphericity, reduced neuronal spike frequency, decreased transient Ca²⁺ concentration, reduced repetitive intracellular Ca²⁺ concentration, decreased amplitude of Ca²⁺ oscillation, reduced Na⁺ current density, decreased action potential, reduced action potential burst trains, reduced firing rate of neurons in whole cell patch clamp recording, reduced number of synapsin puncta or cluster along MAP2-positive processes of neurons, reduced PSD-95 expression level, reduced neuronal networks, reduced astrocyte networks, reduced calcium signaling, reduced calcium wave propagation to surrounding cells upon mechanical stimulation of an individual cell, or disregulated gene expression in the neuronal or glial cell compared to neuronal differentiated iPSC-derived cells or neurons or glial differentiated iPSC-derived cells or astrocytes from an unaffected subject. The presence of one or more of the above being indicative of an increased risk of developing a neurological disease or disorder thereby, identifying a subject with an increased risk of developing a neurological disease or disorder.

In one embodiment, the neurological disease or disorder may be associated with a deficiency or alteration in a glutamatergic pathway affecting neuron and/or glial function.

In another embodiment, the disregulated gene expression may be of any one or more of the genes synapsin-1, PSD-95, Brain-derived neurotrophic factor (BDNF), Nerve Growth Factor Receptor (also referred to as NGFR, CD271; Gp80-LNGFR; TNFRSF16; or p75NTR), Neurotrophin-4 (NT-4), also referred to as neurotrophin-5 (NT-5), NeuN (also referred to as Feminizing Locus on X-3, Fox-3, or Hexaribonucleotide Binding Protein-3), PSD-95 (postsynaptic density protein 95) also referred to as SAP-90 (synapse-associated protein 90), Vesicular glutamate transporter 1 (VGLUT1), VGLut2, Synapsin I (syn1), Insulin-like growth factor 1 (IGF-1), IGF2, Excitatory amino-acid transporter2 (EAAT2), EAAT4, Gamma-aminobutyric acid (GABA) A receptor, alpha 5 (also referred to as GABRA5), Guanine nucleotide-binding protein G(i), alpha-1 subunit (GNAI1), Glutamate receptor 1 (GRIA1), GRIA4, Glutamate [NMDA] receptor subunit epsilon-1 (GRIN2A), Metabotropic glutamate receptor 4 (GRM4), GRM5, GRM7, Bone morphogenetic protein 5 (BMP5), CD40 Ligand (also referred to as gp39 or CD40L), colony stimulating factor 2 (CSF2 or Granulocyte macrophage colony-stimulating factor 2 (also referred to as GM-CSF or sargramostim)), CSF-3 (also referred to as G-CSF and filgrastim)), interferon A4 (IFNA4), interleukin 13 (IL-13), IL-15, IL-23A, IL-3, IL-4, IL-5, Inhibin, beta A, (INHBA), Leukemia inhibitory factor (LIF), tumor growth factor beta-1 (TGF-β1), tumor growth factor beta-2 (TGF-β2), tumor growth factor beta-3 (TGF-β3), Tumor necrosis factor superfamily 12 (TNF-SF12), Tumor necrosis factor superfamily 13 (TNF-SF13B), Tumor necrosis factor superfamily 8 (TNF-SF8), Alpha-taxilin (TXLNA), BMP2, BMP3, BMP4, CD70, IL-10, IL-17B, or IL-18.

The most effective mode of administration and dosage regimen for the therapeutic agents (e.g., NMDA receptor antagonists or modulators of a glutamatergic pathway) depends upon the location, extent, or type of the disease being treated, the severity and course of the medical disorder, the subject's health and response to treatment and the judgment of the treating physician. Accordingly, the dosages of the therapeutic agents should be titrated to the individual subject and/or by the specific medical condition or disease.

By way of example, the interrelationship of dosages for animals of various sizes and species and humans based on mg/m² of surface area is well known. Adjustments in the dosage regimen may be made to optimize inhibition, treatment, or prevention of neurological disease or disorders, e.g., doses may be divided and administered on a daily basis or weekly or biweekly or monthly basis or the dose reduced proportionally depending upon the situation (e.g., several divided doses may be administered daily or proportionally reduced depending on the specific therapeutic situation).

As is well known, the dose of the therapeutic agents required to achieve an appropriate clinical outcome may be further modified with schedule optimization.

Unless defined otherwise, all technical and scientific terms used herein have the same meaning as is commonly understood by one of ordinary skill in the art to which this invention belongs. All patents, applications, published applications and other publications referred to herein are incorporated by reference in their entirety.

ii. Compositions of the Invention

Compositions herein comprise one or more agents provided herein. The agents are, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the agents described above are formulated into pharmaceutical compositions using techniques and procedures well known in the art (see, e.g., Ansel Introduction to Pharmaceutical Dosage Forms, Fourth Edition 1985, 126).

The invention provides compositions and oral or injectable dosage forms comprising a NMDA receptor antagonist(s) including but not limited to any or a combination of 3,5-Dimethyl-tricyclo[3.3.1.13,7]decan-1-amine hydrochloride (Memantine hydrochloride), 1-Aminocyclobutane-1-carboxylic acid (ACBC), D-(−)-2-Amino-5-phosphonopentanoic acid (D-AP5), L-(+)-2-Amino-5-phosphonopentanoic acid (L-AP5), D-(−)-2-Amino-7-phosphonoheptanoic acid (D-AP7), N,N′-1,4-Butanediylbisguanidine sulfate (arcaine sulfate), (R)-4-Carboxyphenylglycine ((R)-4CPG), (S)-4-Carboxyphenylglycine ((S)-4CPG), (E)-(±)-2-Amino-4-methyl-5-phosphono-3-pentenoic acid (CGP 37849), (E)-(±)-2-Amino-4-methyl-5-phosphono-3-pentenoic acid ethyl ester (CGP 39551), [(1S)-1-[[(7-Bromo-1,2,3,4-tetrahydro-2,3-dioxo-5-quinoxalinyl)methyl]amino]ethyl]phosphonic acid hydrochloride (CGP 78608 hydrochloride), cis-4-[Phosphomethyl]-piperidine-2-carboxylic acid (CGS 19755), 7-Chloro-4-hydroxyquinoline-2-carboxylic acid (7-Chlorokynurenic acid), (2R,3S)-β-p-Chlorophenylglutamic acid ((2R,3S)-Chlorpheg), 6-Cyano-7-nitroquinoxaline-2,3-dione (CNQX), 1-[2-(4-Hydroxyphenoxy)ethyl]-4-[(4-methylphenyl)methyl]-4-piperidinol hydrochloride (Co 101244 hydrochloride; PD 174494; or Ro 63-1908), GEXXVAKMAAXLARXNIAKGCKVNCYP (Conantokin-R), GEXXYQKMLXNLRXAEVKKNA (Conantokin-T), 3-((R)-2-Carboxypiperazin-4-yl)-propyl-1-phosphonic acid ((R)-CPP), (RS)-3-(2-Carboxypiperazin-4-yl)-propyl-1-phosphonic acid ((RS)-CPP), D-4-[(2E)-3-Phosphono-2-propenyl]-2-piperazinecarboxylic acid (D-CPP-ene; Midafotel; or SDZ EAA 494), (9α,13α,14α)-3-Methoxy-17-methylmorphinan hydrobromide (Dextromethorphan hydrobromide), 5,7-Dichloro-4-hydroxyquinoline-2-carboxylic acid (5,7-Dichlorokynurenic acid), (±)-1-(1,2-Diphenylethyl)piperidine maleate, α-(4-Chlorophenyl)-4-[(4-fluorophenyl)methyl]-1-piperidineethanol (Eliprodil), 2-Phenyl-1,3-propanedioldicarbamate (Felbamate), N-[2-Amino-6-[[4-fluorophenyl)methyl]amino]-3-pyridinyl]carbamic acid ethyl ester maleate (Flupirtine maleate), 4,6-Dichloro-3-[(1E)-3-oxo-3-(phenylamino)-1-propenyl]-1H-indole-2-carboxylic acid sodium salt (Gavestinel; GV 150526A), (S)-(−)-3-Amino-1-hydroxypyrrolidin-2-one ((S)-(−)-HA-966), (R)-(+)-3-Amino-1-hydroxypyrrolidin-2-one ((R)-(+)-HA-966), (6aS,10aS)-3-(1,1-Dimethylheptyl)-6a,7,10,10a-tetrahydro-1-hydroxy-6,6-dimethyl-6H-dibenzo[b,d]pyran-9-methanol (HU 211; or Dexanabinol), N—(N-(4-Hydroxyphenylacetyl)-3-aminopropyl)-(N′-3-aminopropyl)-1,4-butanediamine (N-(4-Hydroxyphenylacetyl)spermine), (1R*,2S*)-erythro-2-(4-Benzylpiperidino)-1-(4-hydroxyphenyl)-1-propanol hemitartrate (Ifenprodil hemitartrate), (1S*,2 S*)-threo-2-(4-Benzylpiperidino)-1-(4-hydroxyphenyl)-1-propanol hemitartrate (threo Ifenprodil hemitartrate), 2-(2-Chlorophenyl)-2-(methylamino)cyclohexanone hydrochloride (Ketamine hydrochloride), (S)-(+)-2-(2-Chlorophenyl)-2-(methylamino)cyclohexanone hydrochloride ((S)-(+)-Ketamine hydrochloride), trans-2-Carboxy-5,7-dichloro-4-phenylaminocarbonylamino-1,2,3,4-tetrahydroquinoline (L-689,560), 7-Chloro-3-(cyclopropylcarbonyl)-4-hydroxy-2(1H)-quinolinone (L-701,252), 7-Chloro-4-hydroxy-3-(3-phenoxy)phenyl-2(1H)-quinolinone (L-701,324), 4-(4-Chlorophenyl)-4-hydroxy-N,N-dimethyl-α,α-diphenyl-1-piperidinebutanamide hydrochloride (Loperamide hydrochloride), (2R*,4S*)-4-(1H-Tetrazol-5-ylmethyl)-2-piperidinecarboxylic acid (LY 233053), [3S-(3α,4aα,6β,8aα)]-Decahydro-6-(phosphonomethyl)-3-isoquinolinecarboxylic acid (LY 235959), (5R,10S)-(+5-Methyl-10,11-dihydro-5H-dibenzo[a,d]cylcohepten-5,10-imine maleate ((−)-MK 801 maleate), (5S,10R)-(+)-5-Methyl-10,11-dihydro-5H-dibenzo[a,d]cyclohepten-5,10-imine maleate ((+)-MK 801 maleate; MK-801; or Dizocilpine), 2-Amino-2-(2-chlorophenyl)cyclohexanone hydrochloride (Norketamine hydrochloride), D-[[1-(2-Nitrophenyl)ethyl]carbamoyl]-2-amino]-5-phosphonopentanoic acid (NPEC-caged-D-AP5), 4,4′-(Pentamethylenedioxy)dibenzamidine bis-2-hydroxyethanesulfonate salt (Pentamidine isethionate), 1-(1-Phenylcyclohexyl)piperidine hydrochloride (Phencyclidine hydrochloride), 4-(Phosphonomethyl)-2-piperazinecarboxylic acid (PMPA), (2S*,3R*)-1-(Phenanthren-2-carbonyl)piperazine-2,3-dicarboxylic acid (PPDA), (2R*,4S*)-4-(3-Phosphonopropyl)-2-piperidinecarboxylic acid (PPPA; or LY 257883), 2-Amino-N-(1-methyl-1,2-diphenylethyl)acetamide hydrochloride (Remacemide hydrochloride; or FPL 12924AA), 1-[2-(4-Chlorophenyl)ethyl]-1,2,3,4-tetrahydro-6-methoxy-2-methyl-7-isoquinolinol hydrochloride (Ro 04-5595 hydrochloride), (αR,βS)-α-(4-Hydroxyphenyl)-β-methyl-4-(phenylmethyl)-1-piperidinepropanol maleate (Ro 25-6981 maleate), 3,4-Dimethoxy-N-[4-(3-nitrophenyl)-2-thiazolyl]benzenesulfonamide (Ro 61-8048), 4-[3-[4-(4-Fluorophenyl)-1,2,3,6-tetrahydro-1(2H)-pyridinyl]-2-hydroxypropoxy]benzamide hydrochloride (Ro 8-4304 hydrochloride), (S)-α-Amino-2′,4′-dichloro-4-hydroxy-5-(phosphonomethyl)-[1,1′-biphenyl]-3-propanoic acid (SDZ 220-040), (S)-α-Amino-2′-chloro-5-(phosphonomethyl) [1,1′-biphenyl]-3-propanoic acid (SDZ 220-581), N,N′-1,10-Decanediylbisguanidine sulfate (Synthalin sulfate), 3-Chloro-4-fluoro-N-[4-[[2-(phenylcarbonyl)hydrazino]carbonyl]benzyl]benzenesulfonamide (TCN 201), N-(Cyclohexylmethyl)-2-[(5-[(phenylmethyl)amino]-1,3,4-thiadiazol-2-yl thio]acetamide (TCN 213), 1,3-Dihydro-5-[3-[4-(phenylmethyl)-1-2H-benzimidazol-2-one (TCS 46b), nitrous oxide (N₂O), Dextrorphan, Selfotel, Amantadine, Dextrallorphan, Eticyclidine, Gacyclidine, Ibogaine, Ethanol, Methoxetamine, Rolicyclidine, Tenocyclidine, Methoxydine (4-meo-pcp), Tiletamine, Xenon, Neramexane, Etoxadrol, Dexoxadrol, NEFA, Delucemine, 8A-PDHQ, Aptiganel (Cerestat; or CNS-1102), HU-211, Rhynchophylline, 1-Aminocyclopropanecarboxylic acid, 7-Chlorokynurenate, Kynurenic acid, Lacosamide, and/or derivatives thereof, including different acid and/or salt forms and a carrier.

The invention provides compositions and oral or injectable dosage forms comprising a modulator of a glutamatergic pathway which includes, but is not limited to, Acetazolamide (also referred to as N-(5-sulfamoyl-1,3,4-thiadiazol-2-yl)acetamide), a carbonic anhydrase inhibitor), BIX-01294 (having the formula C₂₈H₃₈N₆O₂.3HCl (CAS No. 1392399-03-9, 935693-62-2 (free base)), a G9a histone methyltransferease (G9aHMTase) inhibitor, Zonisamide (also referred to as benzo[d]isoxazol-3-ylmethanesulfonamide), a sulfonamide anticonvulsant, Forskolin (also referred to as (3R,4aR,5S,6S,6aS,10S,10aR,10bS)-6,10,10b-trihydroxy-3,4-a,7,7,10a-pentamethyl-1-oxo-3-vinyldodecahydro-1H-benzo[f]chromen-5-yl acetate), a labdane diterpene, an adenylyl cyclase activator, Tubastatin A (also referred to as N-Hydroxy-4-(2-methyl-1,2,3,4-tetrahydro-pyrido[4,3-b]indol-5-ylmethyl)-benzamide), 7,8 Dihydroxyflavone, Topiramate (also referred to as 2,3:4,5-Bis-O-(1-methylethylidene)-beta-D-fructopyranose sulfamate), a histone deacetylase HDAC6 inhibitor, AR-A014418 (also referred to as N-[(4-Methoxyphenyl)methyl]-N-(5-nitro-2-thiazolyl)urea), a glycogen synthase kinase 3 (GSK3) inhibitor, Amitriptyline (also referred to as 3-(10,11-dihydro-5H-dibenzo[a,d]cycloheptene-5-ylidene)-N,N-dimethylpropan-1-amine), a serotonin-norepinephrine reuptake inhibitor, a 5-HT_{2A}, 5-HT_{2C}, 5-HT₃, 5-HT₆, 5-HT₇, α1-adrenergic, H₁, H₂, H₄, and mACh receptor antagonist, σ1 receptor agonist, a sodium, calcium, and potassium channel blocker, a TrkA and TrkB receptor agonist, LM22A-3, a BDNF mimic, NBI-31772, an insulin-like growth factor 1 (IGF-1) potentiator, ING-135, an ampakine, an α-amino-3-hydroxy-5-methyl-4-isoxazolepropionic acid (AMPA) receptor potentiator, CX-546, LM22A-4, Aripiprazole (also referred to as 7-{4-[4-(2,3-Dichlorophenyl)piperazin-1-yl]butoxy}-3,4-dihydroquinolin-2(1H)-one), a dopamine and serotonin receptor modulator, Hyperzine A or huperzine A (also referred to as (1R,9S,13E)-1-Amino-13-ethylidene-11-methyl-6-azatricyclo[7.3.1.0^{2,7}]trideca-2(7),3,10-trien-5-one), an acetylcholinesterase and NMDA inhibitor, MK-677 (also referred to as Ibutamoren or (R)-1′-(2-methylalanyl-O-benzyl-D-seryl)-1-(methylsulfonyl)-1,2-dihydrospiro[indole-3,4′-piperidine]), a growth hormone secretagogue, Chlormezanone (also referred to as 2-(4-chlorophenyl)-3-methyl-1,1-dioxo-1,3-thiazinan-4-one), a GABA receptor potentiator, Cyctothiazide (also referred to as 3-(bicyclo[2.2.1]hept-5-en-2-yl)-6-chloro-3,4-dihydro-2H-1,2,4-benzothiadiazine-7-sulfonamide 1,1-dioxide), a positive modulator of AMPA receptor, Pioglitazone (also referred to as (RS)-5-(4-[2-(5-ethylpyridin-2-yl)ethoxy]benzyl)thiazolidine-2,4-dione), an agonist of the peroxisome proliferator activated receptor gamma (PPARγ), Memantine (also referred to as 3,5-dimethyltricyclo[3.3.1.1^{3,7}]decan-1 amine or 3,5-dimethyladamantan-1-amine), an NMDA receptor antagonist, a glutamate antagonist, a medium, plasma or extracellular fluid deficient in glutamine or glutamate, a glutamate antagonist, DON (6-Diazo-5-oxo-L-norleucine or (Z,5 S)-5-Amino-1-diazonio-6-hydroxy-6-oxohex-1-en-2-olate; chemical formula C₆H₉N₃O₃ (CAS No. 157-03-9)), a glutamine antagonist, CBX (carbenoxolone, (3β)-3-[(3-carboxypropanoyl)oxy]-11-oxoolean-12-en-30-oic acid, or (2S,4aS,6aS,6bR,8aR,10S,12aS,12bR,14bR)-10-(3-carboxypropanoyloxy)-2,4a,6a,6b,9,9,12a-heptamethyl-13-oxo-1,2,3,4,4a,5,6,6a,6b,7,8,8a,9,10,11,12,12a,12b,13,14b-icosahydropicene-2-carboxylic acid); chemical formula C₃₄H₅₀O₇ (CAS No. 5697-56-3)), a gap junction blocker, Valproic Acid (VPA), a histone deacetylase (HDAC) inhibitor, DAPT (also referred to as LY-374973, N—[N-(3,5-Difluorophenacetyl)-L-alanyl]-S-phenylglycine t-butyl ester), a gamma-secretase inhibitor, Aminoguanidine, an iNOS inhibitor, Dizocilpine (INN) (also referred to as MK-801 or [5R,10S]-[+]-5-methyl-10,11-dihydro-5H-dibenzo[a,d]cyclohepten-5,10-imine), a NMDA receptor antagonist, Curcumin (also referred to as (1E,6E)-1,7-Bis(4-hydroxy-3-methoxyphenyl)-1,6-heptadiene-3,5-dione), Resveratrol (3,5,4′-trihydroxy-trans-stilbene), a curcuminoid, a natural phenol, an anti-inflammatory agent, Ceftriaxone (also referred to as (6R,7R)-7-[(2Z)-2-(2-amino-1,3-thiazol-4-yl)->2-(methoxyimino)acetyl]amino-3-[(2-methyl-5,6-dioxo-1,2,5,6-tetrahydro-1,2,4-triazin-3-yl)thio]methyl-8-oxo-5-thia-1-azabicyclo[4.2.0]oct-2-ene-2-carboxylic acid), Epigallocatechin (also referred to as Gallocatechol or gallocatechin), Gingerol (also referred to as (5)-5-hydroxy-1-(4-hydroxy-3-methoxyphenyl)-3-decanone), Gly-Pro-Glu, and/or Insulin-like growth factor 1 (IGF-1) (also referred to as somatomedin C) and a carrier.

In accordance with the practice of the invention, the composition of the invention may be administered by an intraperitoneal route, enteral route, buccal route, inhalation route, intravenous route, subcutaneous route or intramuscular route.

Further, the compositions of the invention may be formulated as an oral dosage form. The oral dosage form may be a tablet, minitablet, caplet or capsule.

In one embodiment, the compositions of the invention are formulated for single dosage administration. To formulate a composition, the weight fraction of agent is dissolved, suspended, dispersed or otherwise mixed in a selected carrier at an effective concentration such that the treated condition is relieved, prevented, or one or more symptoms are ameliorated.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the active ingredient (also referred to herein as agents) exhibits insufficient solubility, methods for solubilizing agents may be used. Such methods are known to those of skill in this art, and include, but are not limited to, using cosolvents, such as dimethylsulfoxide (DMSO), using surfactants, such as TWEEN™, or dissolution in aqueous sodium bicarbonate. Derivatives of the agents, such as prodrugs of the agents may also be used in formulating effective pharmaceutical compositions.

Upon mixing or addition of the agent(s), the resulting mixture may be a solution, suspension, emulsion or the like. The form of the resulting mixture depends upon a number of factors, including the intended mode of administration and the solubility of the agents in the selected carrier or vehicle. The effective concentration is sufficient for ameliorating the symptoms of the diseases, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the agents or pharmaceutically acceptable derivatives thereof. The pharmaceutically therapeutically active agents and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active agents sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses, which are not segregated in packaging.

Liquid pharmaceutically administrable compositions can, for example, be prepared by dissolving, dispersing, or otherwise mixing an active agents as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Merck Publishing Company, Easton, Pa., 15th Edition, 1975.

Dosage forms or compositions containing active ingredient in the range of 0.005% to 100% (wt %) with the balance made up from non-toxic carrier may be prepared. Methods for preparation of these compositions are known to those skilled in the art. The contemplated compositions may contain 0.001% 100% (wt %) active ingredient, in one embodiment 0.1 95% (wt %), in another embodiment 75 85% (wt %).

### Combination Therapy

In another embodiment, the agents may be administered in combination, or sequentially, with another therapeutic agent. Such other therapeutic agents include those known for treatment, prevention, or amelioration of one or more symptoms of amyloidosis and neurodegenerative diseases and disorders. Such therapeutic agents include, but are not limited to, donepezil hydrochloride (Aricept), rivastigmine tartrate (Exelon), tacrine hydrochloride (Cognex) and galantamine hydrobromide (Reminyl).

### III. Kits

According to another aspect of the invention, kits are provided. Kits according to the invention include package(s) comprising agents or compositions of the invention.

The phrase “package” means any vessel containing agents or compositions presented herein. In preferred embodiments, the package can be a box or wrapping. Packaging materials for use in packaging pharmaceutical products are well known to those of skill in the art. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment.

The kit can also contain items that are not contained within the package but are attached to the outside of the package, for example, pipettes.

Kits may optionally contain instructions for administering agents or compositions of the present invention to a subject having a condition in need of treatment. Kits may also comprise instructions for approved uses of agents herein by regulatory agencies, such as the United States Food and Drug Administration. Kits may optionally contain labeling or product inserts for the present agents. The package(s) and/or any product insert(s) may themselves be approved by regulatory agencies. The kits can include agents in the solid phase or in a liquid phase (such as buffers provided) in a package. The kits also can include buffers for preparing solutions for conducting the methods, and pipettes for transferring liquids from one container to another.

The kit may optionally also contain one or more other compounds for use in combination therapies as described herein. In certain embodiments, the package(s) is a container for intravenous administration. In other embodiments, agents are provided in an inhaler. In still other embodiments agents are provided in a polymeric matrix or in the form of a liposome.

The following examples are presented to illustrate the present invention and to assist one of ordinary skill in making and using the same. The examples are not intended in any way to otherwise limit the scope of the invention.

### EXAMPLES

### Example 1

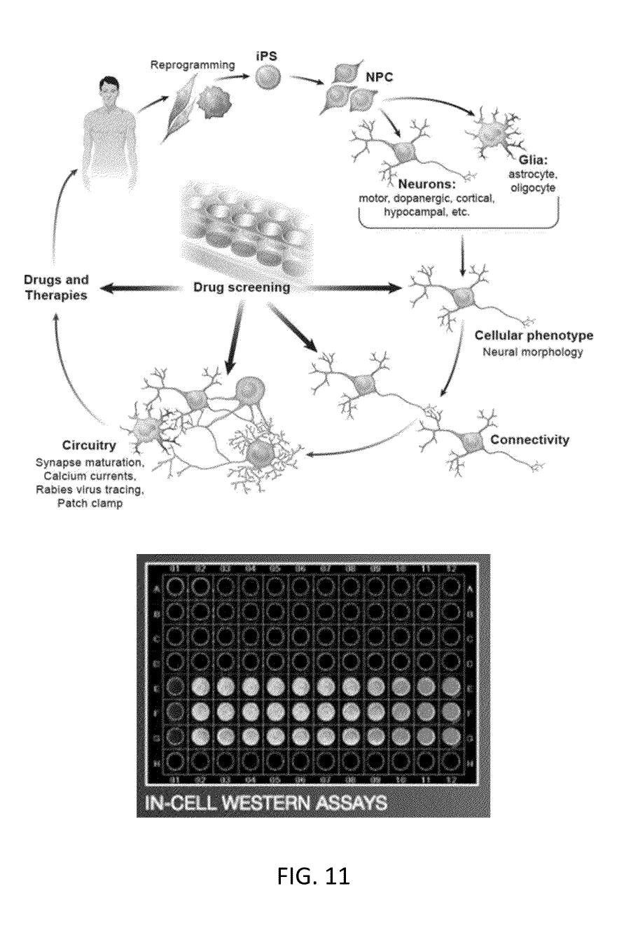

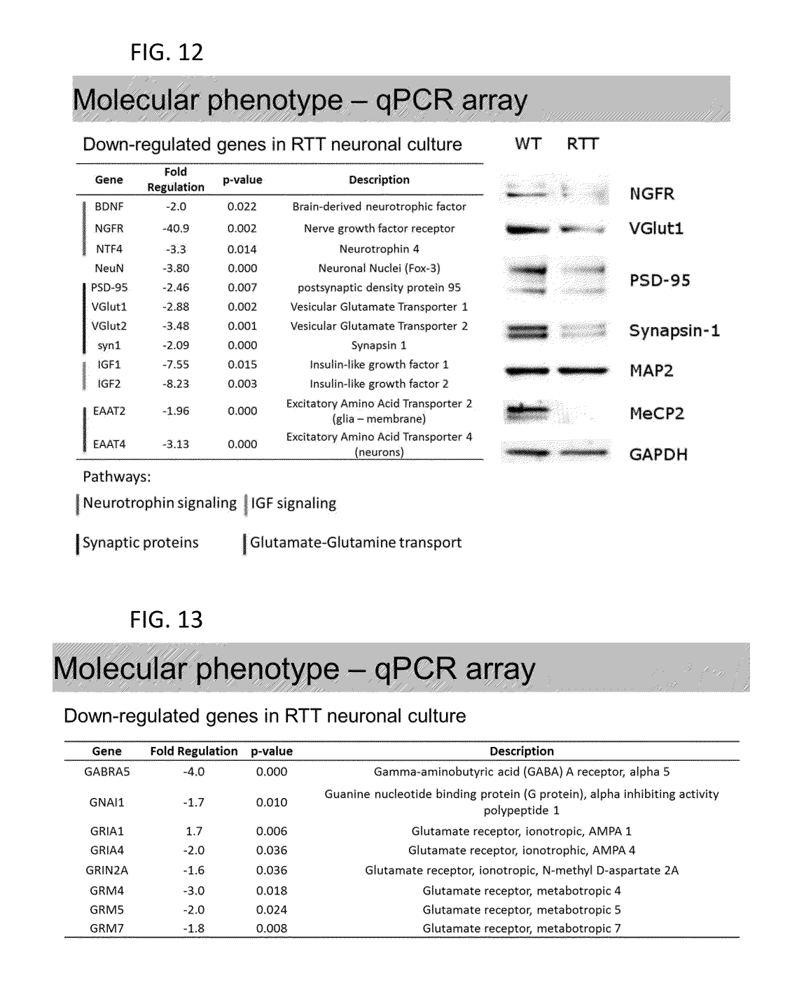

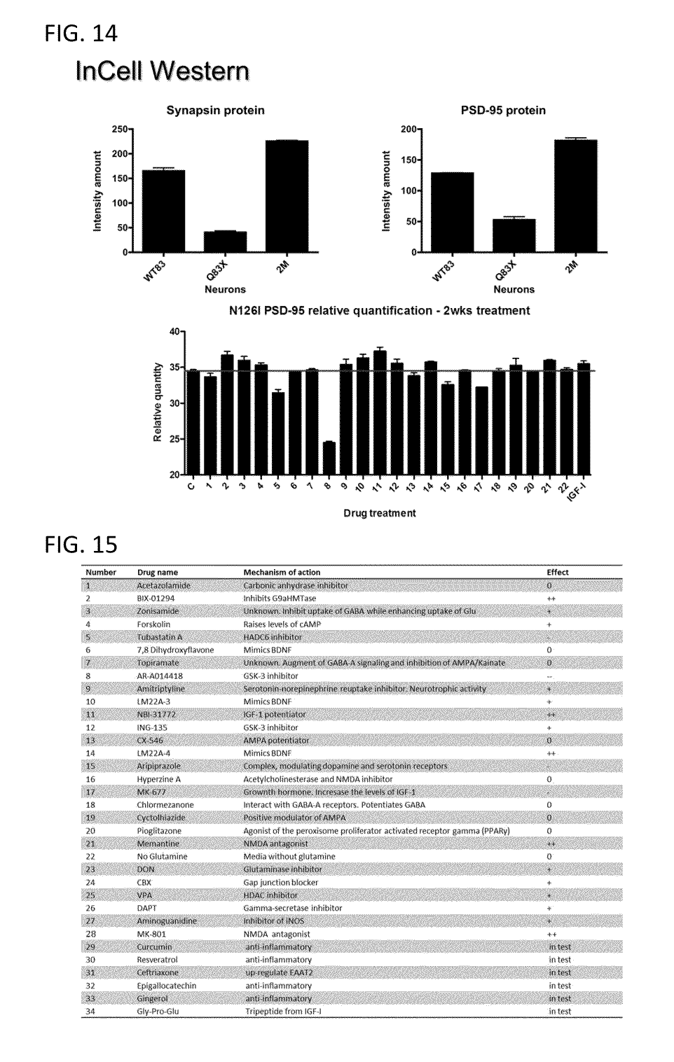

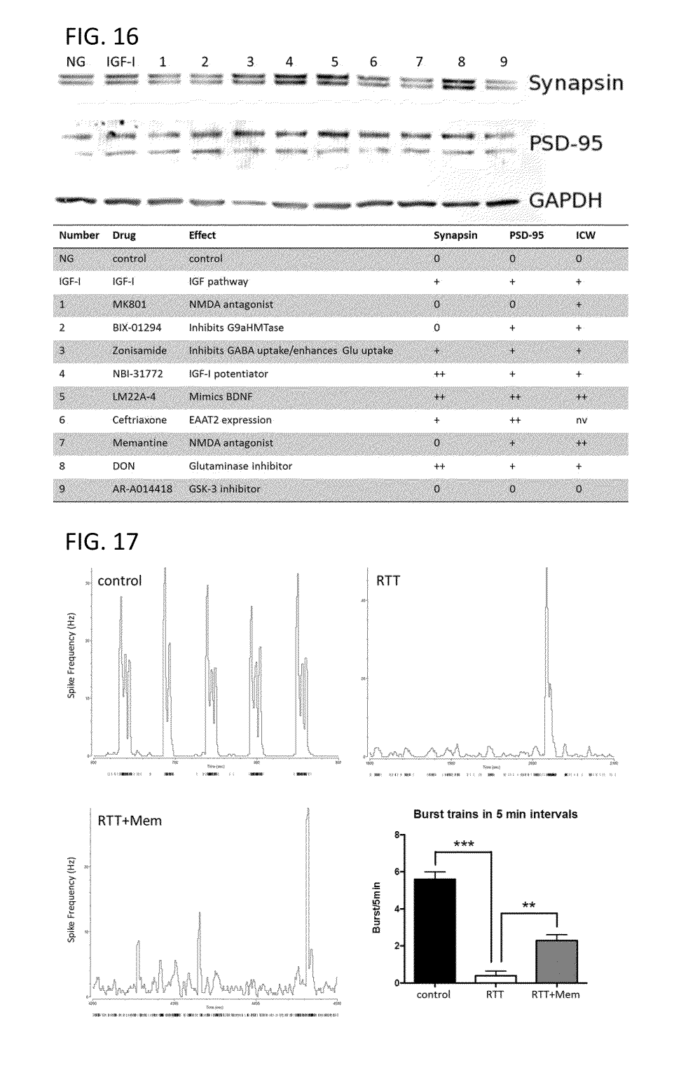

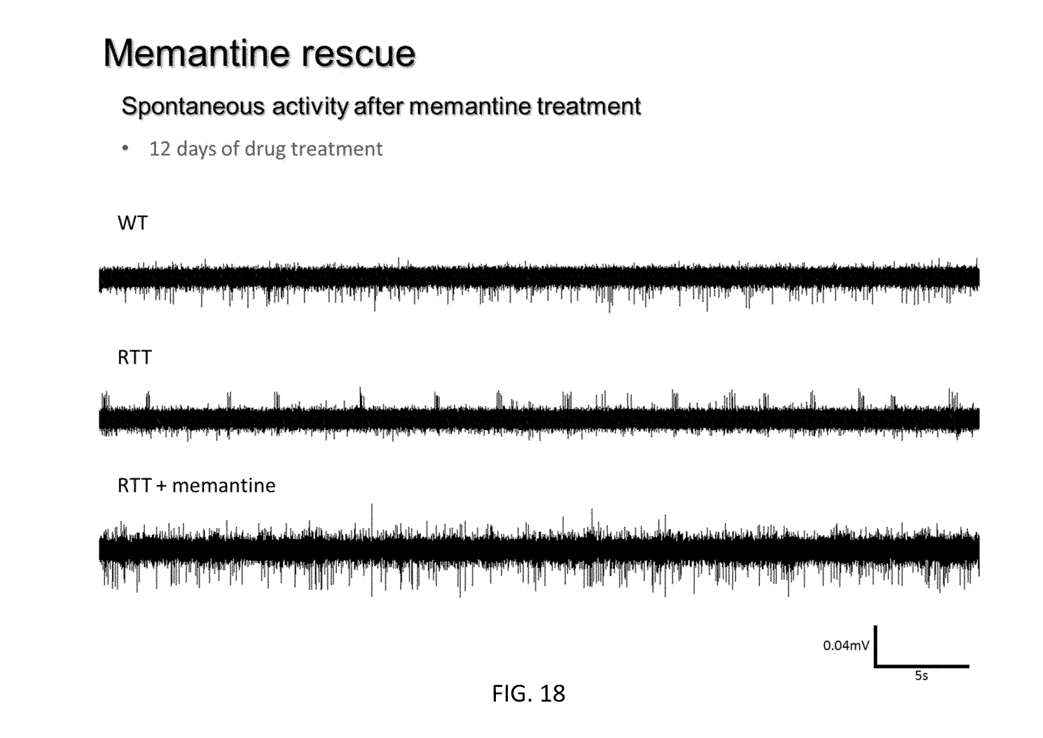

Fibroblasts from patients are reprogrammed to a pluripotent state and further differentiated into neuro progenitor cells (NPCs). These NPCs can be expanded in appropriated culture conditions and, under the right signals, induced to differentiated into postmitotic neurons (see schematic; FIG. 11). Several adjustments were done on the neuronal differentiation protocol, reducing by half the time needed to generate neurons from NPCs. Such adjustments include specific factors and cell manipulations at different stages of the protocol. At this stage, disease neurons can reveal a phenotype that is related to the mutations on the MeCP2 gene. We focused on synapse quantification. Instead of manual synapse counting, we are using an Odyssey machine that can estimate the number of synapses by detecting specific wavelengths in a 96- or 24-well plates (see FIG. 11). Our preliminary data indicates that we are able to detect small alterations in response to IGF1, a drug known to increase synapse number in human neurons (Marchetto et al., Cell, 2010). Alternatively, we are using specific synaptic tools to visualize synaptogeneses in real time that could be detected by any time-lapse fluorescence microscope.

### REFERENCES FOR EXAMPLE 1

1. Marchetto M C, Carromeu C, Acab A, Yu D, Yeo G W, Mu Y, Chen G, Gage F H, Muotri A R. A model for neural development and treatment of Rett syndrome using human induced pluripotent stem cells. Cell. 2010 Nov. 12; 143(4):527-39.
2. Itzhaki I, Maizels L, Huber I, Zwi-Dantsis L, Caspi O, Winterstern A, Feldman O, Gepstein A, Arbel G, Hammerman H, Boulos M, Gepstein L. Modelling the long QT syndrome with induced pluripotent stem cells. Nature. 2011 Mar. 10; 471(7337):225-9. Epub 2011 Jan. 16.
3. Vogel G. Stem cells. Diseases in a dish take off Science. 2010 Nov. 26; 330(6008):1172-3.
4. Inoue H, Yamanaka S. The Use of Induced Pluripotent Stem Cells in Drug Development. Clin Pharmacol Ther. 2011 Mar. 23.
5. Plath K, Lowry W E. Progress in understanding reprogramming to the induced pluripotent state. Nat Rev Genet. 2011 April; 12(4):253-65.
6. Li Z K, Zhou Q. Cellular models for disease exploring and drug screening. Protein Cell. 2010 April; 1(4):355-62. Epub 2010 May 8.

### Example 2

### Experimental Procedures

Patient Ascertainment. Patient F2749-1 (TRPC6-Mutant):

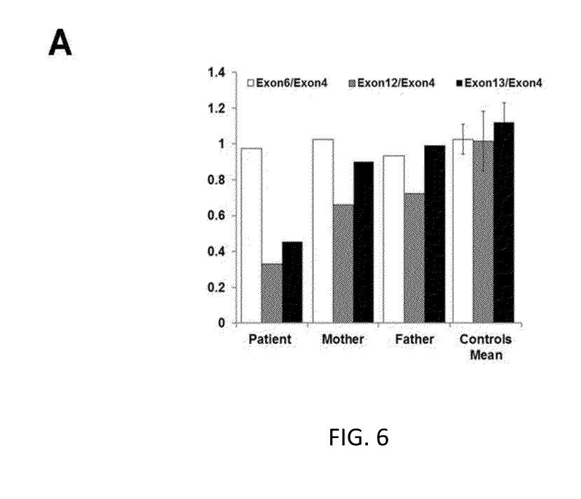

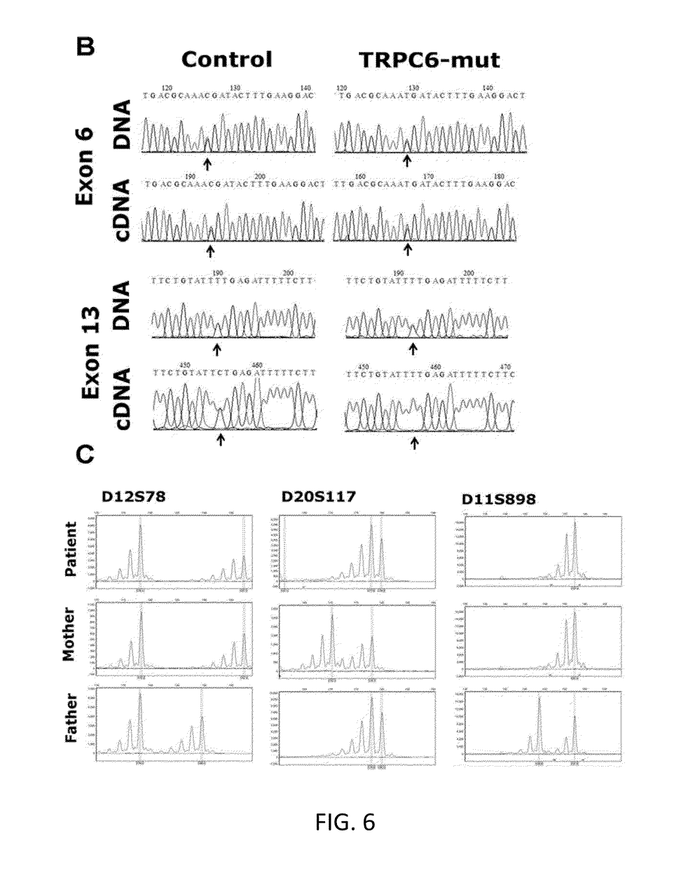

The 8 year old proband is the only child of non-consanguineous healthy parents. He was born at term after an uncomplicated pregnancy with no malformations recognized at birth. He was noted to have delayed motor skills development and poor social responsiveness and was brought to medical attention at 2 years of age. His hearing was tested and found to be normal. He did not suffer from any other chronic medical conditions and there was no history of head trauma or seizure. On examination the patient met DSM-IV criteria for autistic disorder and the diagnosis was supported by administration of the Childhood Autism Rating Scale (CARS). Electroencephalogram and magnetic resonance imaging were normal. The patient did not have dysmorphic features, except for synophrys, which is also present in other members of the father's family. Molecular test for Fragile-X Syndrome was normal. Karyotype analysis revealed a balanced translocation (46, XY, t[3;11][p21;q22]) in the proband, which was not found in parents. Parenthood was confirmed by genotyping of microsatellite markers (FIG. 6C). This project was approved by the Ethics Committee of the Institutes where the study was conducted. After a complete description of the study, written informed consent was signed by the parents.

### Analysis of Genomic Copy Number Variations.

Genomic DNA was hybridized to the HumanHap300 Genotyping BeadChip from Illumina, according to manufacturer's protocol, to detect possible copy number variations (CNVs) present in the patient. The data were analyzed using PennCNV (Wang et al., 2007) and QuantiSNP (Colella et al., 2007) software and results were compared to the database of genomic variants (http://projects.tcag.ca/variation/) in order to classify the identified CNVs as rare or common variants.

### Fluorescent In Situ Hybridization.

Chromosomes for Fluorescent In Situ Hybridization (FISH) analysis were prepared from colchicine-treated lymphocytes of the proband. Bacterial Artificial Chromosomes (BACs) covering the genomic regions of interest were selected from the RPCI-11 library (Roswell Park Cancer Institute) via UCSC genome browser (http://genome.ucsc.edu/, assembly March 2006, NCBI36/hg18). BACs were fluorescently labeled using nick translation and hybridized to the metaphase spreads using standard protocols (Lichter et al., 1990).

### Isolation and Culture of Human Dental Pulp Cells.

DPSC lineages were obtained as described elsewhere (Beltrao-Braga et al., 2011). Briefly, dental pulp tissues were digested in a solution of 0.25% trypsin for 30 minutes at 37° C. The cells were cultivated in DMEM/F12 media (Gibco) supplemented with 15% fetal bovine serum (Hyclone, Tex.), 1% penicillin/streptomycin and 1% non-essential amino acids and maintained under standard conditions (37° C., 5% CO₂). DPSC control lineages used for the whole-genome expression analysis were donated by Dr. Daniela Franco Bueno and Gerson Shigueru Kobayashi from University of Sao Paulo. One of the DPSC control lineages used for iPSC generation was a kind gift from Dr. Songtao Shi (University of Southern California).

### RNA Extraction.

RNA samples were extracted from lymphocytes, DPCs and iPSCs by Trizol reagent (Invitrogen, CA) and treated with Turbo DNase-free (Ambion). Sample concentration and quality were evaluated using Nanodrop 1000 and gel electrophoresis.

### Microarray Studies.

For microarray experiments, 100 ng of RNA was reverted to cDNA, amplified, labeled and hybridized to the Human Gene 1.0ST chip from Affymetrix, following manufacturer's protocol. The chips were scanned by GeneChip® Scanner 3000 7G System and a quality control was processed by Affymetrix® Expression Console™ Software. The data were normalized using the Robust Multi-array Average (RMA) method (Irizarry et al., 2003), and the differentially expressed genes were selected with the SAM method (Significance Analysis of Microarrays (Tusher et al., 2001) or RankProd. For the selection of DEGs, we used a p-value<0.05 adjusted for the FDR (False Discovery Rate) and 3,000 permutations. Functional annotation, canonical pathways and networks analysis were performed using Ingenuity Pathways (http://www.ingenuity.com/). CREB target genes database (http://natural.salk.edu/CREB/search.htm, (Zhang et al., 2005)) was used in order to determine whether the DEGs found are regulated by the transcription factor CREB.

Gene Expression Analyses by qPCR.

RNA samples were reversed transcribed into cDNA using the Super Script III First Strand Synthesis System (Invitrogen, CA) according to manufacturer's instructions. Reactions were run on an Applied Biosystem 7500 sequence detection system using Syber-green master mix (Applied Biosystems, CA). Primers were designed using PrimerExpress v. 2.0 software (Applied Biosystems, CA) and specificity was verified by melting curve analysis on 7500 System SDS v. 1.2 Software (Applied Biosystems, CA). Sequences of the primers are described in Table S6.

TABLE S6
Sequence of the primers used in qPCR experiments.

| Gene | Primer F | Primer R |
|---|---|---|
| GAPDH | TGCACCACCAACTGCTTAGC | GGCATGGACTGTGGTCA |
| | | TG |
| HPRT1 | TGACACTGGCAAAACAATGC | GTCCTTTTCACCCAGCA |
| | | AGC |
| HMBS | GGCAATGCGGCTGCAA | GGTAAGGCACGCGAATC |
| | | AC |
| SDHA | TGGGAACAAGAGGGCATCTG | CCACCACTGCATCAAAT |
| | | TCATG |
| TRPC6 (4) | TGGTCCACGCATTATCTTCCC | TTACGACAGCAGACAAT |
| | | GGCG |
| TRPC6 (6) | GCATGCTTCCAAAGCCCAGAG | CCACTTTATCCTGGCCA |
| | | AATTG |
| TRPC6 (12) | TGTACTGCAGGCCCAGATAGA | GGAGTTCATAGCGGAGA |
| | | CTTG |
| TRPC6 (13) | CAAGTCTCCGCTATGAACTCC | CCTCTTGATTTGGTTCC |
| | | ATGGA |
| CDH6 | AGCTGCAGTTTCAGCCGCGA | AGGATATCTCTGCTCGC |
| | | CTTCCTG |
| CLDN11 | CTGGGTCTGCCGGCCATTTT | GCGCAGAGCCAGCAGAA |
| | | TGA |
| SEMA3A | CACTGCAAAGAGACGCACAAG | GCTGTGGCCATGGTGAT |
| | | TATC |
| EPHA4 | CAAGATACAGTGTGGCACTGG | GCTTCGCTCATTCTGAT |
| | | CCTTC |
| NPTX1 | ACGAGCTGGTCCTCATTGAGT | TGCCACTTGCCATCATT |
| | | GATG |
| MAP2 | CCTTTGAGAACACGACACAAC | GCCTTTGCTTCATCTTT |
| | | CCGT |
| INA | GGAACACCAAGAGTGAGATG | GCCTTCCAGCAGTTTCC |
| | | TGTA |
| PCDH10 | GGACTGCTGACTAATACGCGA | GCAAATCATGCTGCTTC |
| | | AGGT |

Quantitative analysis was performed using the comparative threshold cycle method (Livak and Schmittgen, 2001). GeNorm (www.medgen.ugent.be/genorm/) was used to determine the stability of the reference genes GAPDH, HPRT1, SDHA and HMBS generate a normalization factor for the expression values of the target genes. The principles of analysis of geNorm have been described (Vandesompele et al., 2002). Microarray validation was performed using the one-tailed unpaired t-test with Welch's correction to compare the qPCR expression values obtained for the patient and controls. A concentration of 10 μM of hyperforin was used to treat DPSC of a control sample for 15 min, 30 min, 1, 3, 6, 24, and 48 hours. The samples were run in triplicate and the results were normalized by the values obtained for a non-treated sample.

### Western Blotting.

Rabbit anti-TRPC6 antibody (ProScience, 1:250), Rabbit anti-CREB (Cell Signaling, 1:500), Rabbit anti-P-CREB (Cell Signaling, 1:500) and Mouse anti-β-actin (Ambion, 1:5000) were used as primary antibodies. Horseradish-peroxiadase-conjugated goat anti-Rabbit and goat anti-Mouse (Promega, 1:2000) were used as secondary antibodies. ECL Plus (Amersham) was used for signal detection. Signal intensities were measured using ImageJ and semi quantitative analysis of p-CREB signal intensity was corrected with respect to CREB/β-actin relative quantification. Paired t-test analysis with a p-value<0.05 was used in the comparison of control and patient p-CREB signal intensity normalized data.

### Cellular Reprogramming.

Induced pluripotent stem cells (iPSC) were obtained from DPSC of the patient and a control. Briefly, overexpression of OCT4, SOX2, KFL4 and MYC were induced in DPSC through the transduction of these cells with retrovirus containing these genes (Takahashi et al., 2007). Two days after the transduction, the cells were transferred to a co-culture with murine embryonic fibroblasts (mEFs), maintained with DMEM/F12 (Invitrogen, CA), 20% Knockout Serum Replacement (Invitrogen, CA), 1% non-essential amino acids and 100 μM beta-mercaptoethanol and treated with 1 mM of Valproic acid (Sigma) for 5 days. The iPSC colonies were identified after approximately 2 weeks in this culture system and were transferred to matrigel (BD Biosciences) coated plates and maintained with mTeSR media (Stem Cell Technologies).

### Immunocytochemistry.

Cells were fixed with PBS containing 4% paraformaldehyde for 10 minutes and then incubated at room temperature for 1 hour in a blocking solution containing 5% of donkey serum and 0.1% Triton X-100. The incubation with the primary antibodies was done overnight at 4° C., followed by incubation with secondary antibodies (Jackson Immunoresearch) for 1 hour at room temperature. Images were captured with a Zeiss microscope. Primary antibodies used: Tra-1-81 (1:100, Chemicon); Nanog and Lin28 (1:500, R&D Systems); Sox2 (1:250; Chemicon); human Nestin (1:100, Chemicon); Tuj1 (1:500, Covance); MAP2 (1:100; Sigma); VGLUT1 (1:200, Synaptic Systems); GABA (1:100, Sigma); Musashi (1:200, Abcam); Ctip2 (1:200, Abcam) and Tbrl (1:200, Abcam).

### Teratoma Formation.

iPSC colonies from five semi-confluent 100 mm dishes (1-3×10⁶ cells) were harvested after treatment with 0.5 ng/ml dispase, pelleted and suspended in 300 μL of matrigel. The cells were injected subcutaneously in nude mice. Five to six weeks after injection, teratomas were dissected, fixed overnight in 10% buffered formalin phosphate and embedded in paraffin. Sections were stained with hematoxylin and eosin for further analysis. Protocols were previously approved by the University of California San Diego Institutional Animal Care and Use Committee.

### Fingerprinting and Karyotype.

Standard G-banding karyotype and DNA fingerprinting analysis were performed by Cell Line Genetics (Madison, Wis.).

### Neuronal Differentiation.

The iPSC colonies were plated on matrigel (BD Biosciences) coated plates and kept for 5 days with mTSeR media (Stem Cell Technologies). On the fifth day, the media was changed to N2 media (DMEM/F12 media supplemented with 1× N2 supplement (Invitrogen) and 1 μM of dorsomorphin (Tocris). After two days in this condition, the colonies were removed from the plate and cultured in suspension as Embryoid Bodies (EBs) for 2-3 weeks using N2 media with dorsomorphin during the entire procedure. The EBs were then gently dissociated with accutase (Gibco) and plated on matrigel-coated dishes and maintained with NBF media (DMEM/F12 media supplemented with 0.5×N2, 0.5× B7 supplements, 20 ηg/mL of FGF and 1% penicillin/streptomycin). The rosettes that emerged after 3 or 4 days were manually selected, gently dissociated with accutase and plated in dishes coated with 10 μg/mL poly-ornithine and 5 μg/mL laminin. This NPC population was expanded using the NBF media. To differentiate the NPCs into neurons, cells were re-plated with 10 μM of ROCK inhibitor (Y-27632, Calbiochem) in the absence of FGF, with regular media changes every 3 or 4 days.

### Ca²⁺ Influx Studies.

Intracellular Ca²⁺ levels were monitored using Fluo-4AM. The cells were incubated for 45 minutes at 37° C. with 2.5 μM of Fluo4-AM and superfused for 5 minutes with HBSS buffer before the beginning of the recording. 10 μM hyperforin (a kind gift from Dr. Willmar Schwabe GmbH & Co, Karlsruhe, Germany) was used in combination with 100 μM FFA (Sigma-Aldrich) for TRPC6 (human transient receptor potential cation channel, subfamily C, member 6, NCBI Gene ID: 7225 incorporated by reference herein) activation. The images were taken at 6 seconds intervals for 30 minutes using a Biorad MRC 1024 confocal system attached to an Olympus BX70 microscope at 6 seconds intervals during 30 minutes. The drugs were applied at the third minute using a perfusion system. A triplicate of each individual was analyzed. The average fluorescence of individual cells was quantified and normalized to the resting fluorescence level for each cell. The plugins MultiMeasure and MeasureStacks from ImageJ software were used to measure fluorescence intensity.

### Cell Cycle Analysis.

One million NPCs were harvested to single cell suspension with PBS washing buffer (PBS and 1% serum), then fixed in 75% EtOH for at least 2 hours at 4° C. After washing twice with washing buffer, cells were stained using 200 μL of propidium iodine (PI) solution (20 mg/mL propidium iodide, 200 mg/mL RNase A and 0.1% Triton X-100). Multiple NPC samples from TRPC6-mutant patient and controls were analyzed by fluorescence-activated cell sorting (FACS) on a Becton Dickinson LSR1 and cell cycle gating was examined using FLOWJO-Flow Cytometry Analysis Software.

### Quantification of Neuronal Morphology and Synaptic Puncta.

Neuronal tracing was performed on neurons for which that the shortest dendrite was at least three-times longer than the cell soma diameter, using a semi-automatic ImageJ plug-in (NeuroJ). Spines and VGLUT1 puncta were quantified after three-dimensional reconstruction of z-stack confocal images. Only neurons with spines were scored. Pictures were taken randomly for each individual and from two different experiments, using at least two different clones. Quantification was done blindly to the genotype of the cells. All experiments were performed with independent clones and different controls. Table S3 summarizes the subjects and clones used for each experiment. For the rescue experiments, 10 ηg/mL of IGF1 (Peprotech) was added to neuronal cultures for 2 weeks.

TABLE S3
Summary of the iPSC subjects and clones (C) utilized for each experiment.
Numbers represent experimental replications for each individual clone. The clones utilized in
neuronal differentiation experiments were determined by availability at the end time-point.

| | | | | | | | | | | F1850-1 | | |
|---|---|---|---|---|---|---|---|---|---|---|---|---|
| | F2749-1 | | | USC1 | | | P603 | | | (idiopathic | | |
| | (TRPC6-mut) | | | (control) | | | (control) | | | ASD) | | |
| Study/cell line | DPSC | C4 | C6 | DPSC | C1 | C2 | DPSC | C1 | C2 | DPSC | C1 | C2 |
| DPSC microarray | 3 | | | 3 | | | | | | | | |
| DPSC microarray | 3 | | | | | | | | | | | |
| study validation | | | | | | | | | | | | |
| (qPCR) | | | | | | | | | | | | |
| Hyperforin | | | | 3 | | | | | | | | |
| treatment gene | | | | | | | | | | | | |
| expression | | | | | | | | | | | | |
| CREB | 3 | | | 3 | | | | | | | | |
| phosphorylation | | | | | | | | | | | | |
| Pluripotency | | 1 | 1 | | 1 | 1 | | 1 | 1 | | 1 | 1 |
| assays | | | | | | | | | | | | |
| iPSC microarray | 3 | 3 | 3 | 3 | 3 | | | | | | | |
| NPC cell cycle | | 3 | 3 | | 3 | 3 | | | | | | |
| NPC Ca2+ influx | | 3 | | | 3 | | | | | | 3 | |
| Neuronal | | 3 | 2 | | 3 | 2 | | | 3 | | 3 | 2 |
| arborization | | | | | | | | | | | | |
| VGlut1 puncta | | 3 | 2 | | 3 | 2 | | | 3 | | 3 | 2 |
| Electrophysiology | | 3 | | | 3 | | | | | | | |
| Spine density | | 3 | 2 | | 3 | 2 | | | 3 | | 3 | 2 |

### Construction and Characterization of Retroviruses.

Self-inactivating murine oncoretroviruses were engineered to express short-hairpin RNAs (shRNAs) under the U6 promoter and green fluorescent protein (GFP) or the Discosoma sp. red fluorescent protein DsRed under the Ef1 alpha promoter. shRNAs against TRPC6 and a non-silencing scrambled control shRNA were cloned into retroviral vectors, as previously described (Kim et al., 2009). The following shRNA sequences were selected and cloned into retroviral vectors:

shRNA-control: 5′-TTCTCCGAACGTGTCACGT-3′
shRNA-TRPC6-1: 5′-TCGAGGACCAGCATACATG-3′
shRNA-TRPC6-3: 5′-CTCAGAAGATTATCATTTA-3′

For rescue experiments, a resistant form of murine TRPC6 (TRPC6-WT^{R}) was engineered to harbor 6 silent mutations in the region targeted by shRNA-TRPC6-1. The targeting sequence of TRPC6 was mutated from AAT CGA GGA CCA GCA TAC ATG to AAC CGC GGC CCT GCT TAT ATG by site directed mutagenesis. The resistant form of TRPC6 was cloned into a retroviral vector driven by the Ubiquitin promoter followed by a bicistronic expression of GFP and a WPRE stabilization sequence. The specificity and efficiency of shRNA-control, shRNA-TRPC6-1, shRNA-TRPC6-3, and the TrpC6-WT constructs were verified by co-transfection into HEK 293 cells. Cell lysates were collected and analyzed on a western blot probed with anti-TRPC6 antibodies (Sigma).

In Vivo Stereotaxic Injection of Engineered Retroviruses into the Dentate Gyrus of Adult Mouse Hippocampus.

High titers of engineered retroviruses were produced by cotransfection of retroviral vectors and vesicular stomatitis viral envelope into 293 GP cell line as described (Duan et al., 2007). Supernatants were collected 24 hours post transfection, filtered through 45 micron filters, and ultracentrifugated. Viral pellet was dissolved in 14 μl PBS and stereotaxically injected into the hilus of anesthetized mice at four sites (0.5 μl per site at 0.25 μl/min). The following coordinates were used, posterior=2 mm from Bregma, lateral=±1.6 mm, ventral±2.5 mm; posterior=3 mm from Bregma, lateral=±2.6 mm, ventral=±3.2 mm. Adult C57BL/6 mice (6-8 weeks old, female) were used for the study. All procedures followed the institutional guidelines.

### Immunostaining and Confocal Analysis.

Coronal brain sections (40 μm) thick were prepared from retrovirally injected mice. Images of GFP⁺ cells were acquired on a META multiphoton confocal system. Neuronal positioning was analyzed by taking a single section confocal image of GFP⁺ cell body stained with DAPI and assigning it to one of the 4 domains as illustrated. A minimum of 10 GFP⁺ cells were randomly picked from the each animal and at least 3 animals were used under each experimental condition as previously described (Kang et al., 2011). Statistical significance was determined using ANOVA. Dendritic development was analyzed by taking 3 dimensional reconstruction of the entire dendritic tree made from Z-series stacks of confocal images. Images were converted into 2 dimensional projections for analysis of dendritic length and branch number using the NIH ImageJ software and the NeuronJ plugin, as described (Kang et al., 2011). Scholl analysis was performed by counting the number of dendritic crossings at a series of concentric circles at 10 μm intervals from the cell body using the Scholl analysis plugin.

### Slice Electrophysiology.

Mice housed in standard conditions were anesthetized at 3 weeks post retroviral injection and acute coronal slices were prepared as previously described (Ge et al., 2006). Brains were removed into an ice cold cutting solution containing: 110 mM choline chloride, 2.5 mM KCl, 1.3 mM KH₂PO₄, 25 mM NaHCO₃, 0.5 mM CaCl₂, 7 mM MgCl₂, 10 mM dextrose, 1.3 mM sodium ascorbate, 0.6 mM sodium pyruvate, 5 mM kynurenic acid. Slices were cut 300 μm thick on a vibratome (Leica VT1000S) and transferred to a chamber with ACSF: 125 mM NaCl, 2.5 mM KCl, 1.3 mM KH₂PO₄, 25 mM NaHCO₃, 2 mM CaCl₂, 1.3 mM MgCl₂, 1.3 mM sodium ascorbate, 0.6 mM sodium pyruvate, 10 mM dextrose (pH 7.4, 320 mOsm), saturated with 95% O₂, 5% CO₂ at 35° C. for 20 minutes, and transferred to room temperature at least 45 minutes prior to placement in the recording chamber. Slices were maintained at room temperature and used for the following 4 hours. Electrophysiological recordings were performed at 34° C. Microelectrodes (4-6 MΩ) were filled with the following solution: 120 mM potassium gluconate, 15 mM KCl, 4 mM MgCl₂, 0.1 mM EGTA, 10.0 mM HEPES, 4 mM MgATP, 0.3 mM Na₃GTP, 7 mM phosphocreatine (pH7.4, 300 mOsm). The whole-cell patch-clamp configuration was used in the current-clamp mode. About 10-20 Giga-ohm seals were obtained with borosilicate glass microelectrodes. The electrophysiological recordings were obtained at 32-34° C. Neurons and dendrites were visualized by differential interference contrast microscopy. Data were collected using an Axon Instruments 200B amplifier and acquired via a Digidata 1322A at 10 kHz.

Electrophysiology recordings on cultured human iPSC-derived neurons.

Whole-cell patch clamp recordings were performed from cells cultured in the absence of astrocytes after approximately 6 weeks of differentiation. Before recordings, the growth media was removed and replaced with a bath solution comprised of (in mM): 130 NaCl, 3 KCl, 1 CaCl₂, 1 MgCl₂, HEPES, and 10 glucose (pH 7.4) at room temperature (22-24° C.). Electrodes for whole cell recording were pulled on a Flaming/Brown micropipette puller (Model P-87, Sutter Instrument, Novato, Calif.) from filamented borosilicate capillary glass (1.2 mm OD, 0.69 mm ID, World Precision Instruments, Sarasota, Fla.). The electrodes were fire-polished, and resistances were typically 2-5 MΩ for voltage-clamp experiments and 7-9 MΩ for current-clamp experiments. The pipette solution contained (in mM): 138 KCl, 0.2 CaCl₂, 1 MgCl₂, 10 HEPES (Na⁺ salt), and 10 EGTA, (pH 7.4). The osmolarity of all solutions was adjusted to 290 mOsM. All chemicals were purchased from Sigma-Aldrich (St. Louis, Mo.) except MgCl₂ (J.T. Baker, Phillipsburg, N.J.). Current traces in voltage clamp were leak-subtracted. Liquid junction potentials were nulled for each individual cell with the Axopatch 1C amplifier (Molecular Devises, Sunnyvale, Calif.).

### Behavioral Tests in Mice.

The three-chamber test was used to evaluate the social behavior of TRPC6 wild type (WT), heterozygous (HET) and knockout (KO) mice. To evaluate repetitive behavior, mice were initially observed during 10 minutes in the dark and the time spent in grooming and freezing behavior was measured. After 5 minutes of habituation in a light condition, a small cage with a never-met animal was introduced to one side of the box and an empty cage was introduced to the other side. The time spent in each chamber and the time spent during nose-to-nose interaction between the animals was measured. Adult mice (6-8 weeks old, male) in a C57BL/6 background were used for the study. At least 12 animals per group were utilized in biological replicates. Experimenter was blind to the genotypes. The data were analyzed using the non-parametric ANOVA test Kruskal-Wallis. All procedures followed the institutional guidelines.

### Mutation Screening of TRPC6.

### Cohorts:

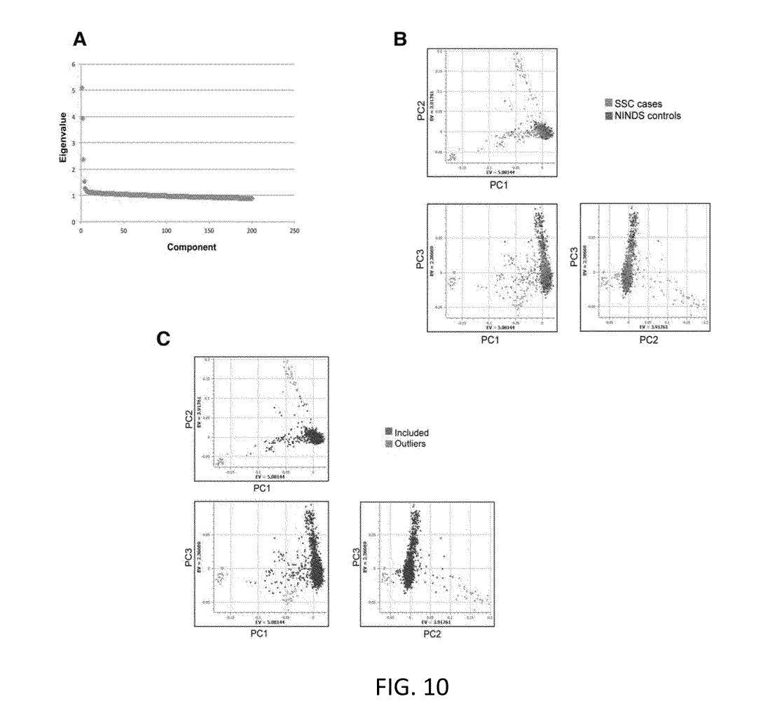

Clinical characteristics of the Simons Simplex Collection (SSC) were previously described in detail (Fischbach and Lord, 2010). The following exclusion criteria were used to filter the cases: 1) ineligible/ancillary status as per SSC Family Distribution List v13, 2) missing genotyping data, 3) genotyping call rate <95%, 4) discrepancy of genotyping data with recorded gender, 5) Mendelian inconsistencies or cryptic relatedness (up to and including second degree relatives), and 6) non-European ancestry (see Supplementary Experimental Procedures; FIG. 10). 1041 of 1195 cases passed these quality checks and were included in the final case cohort. The National Institute of Neurological Disorders and Stroke (NINDS) Neurologically Normal Caucasian Control Panel of unrelated adult controls do not have a personal or family history (first degree relative) of neuropsychiatric illness (http://ccr.coriell.org/Sections/Collections/NINDS/DNAPanels. aspx?Pgld=195 &coll=ND). Of 953 samples from DNA panels NDPT020, 079, 082, 084, 090, 093, 094, 095, 096, 098, and 099, 942 passed the quality control checks above. Additional sequence data for TRPC6 were derived from unrelated northern European (NE) adults present in an exome-sequencing database in our laboratory. Genotyping and whole-exome data were obtained for 2076 individuals, 1930 of which passed the above quality control checks.

### Mutation Screening:

For 1031 SSC cases and all 942 NINDS controls, amplification of the coding exons and splice sites was performed on lymphoblastoid cell line-derived genomic DNA via multiplex PCR using RainDance technology (Lexington, Mass., USA; see Table S5 for TRPC6 primer sequences).

TABLE S5
PCR primers covering all coding regions of TRPC6, designed by RainDance.

| | | | | | | | | | amplicon_ |
|---|---|---|---|---|---|---|---|---|---|
| Exon | sense_ | sense_ | sense_ | | antisense_ | antisense | antisense | antisense_ | length |
| Number | primer | start | sequence | sense_Tm | primer | start | sequence | Tm | (bp) |
| 1 | 646_e1_ | chr11: | GAGGAGCAA | 58.468 | 646_e1_t0_R | chr11: | CTTAAGTGGT | 58.008 | 590 |
| | t0_L | 100959059 | ACCTAGACAA | | | 100959648 | GACTTTTCCC | | |
| 2 | 645_e1_ | chr11: | TCGAGAGAG | 57.9 | 645_e1_t3_R | chr11: | TTCTATCTGAA | 58.033 | 583 |
| | t3_L | 100880430 | GTTTTCTTTCT | | | 100881012 | TGGCACAGA | | |
| 2 | 645_e1_ | chr11: | GAAATAATC | 57.931 | 645_e1_t2_R | chr11: | AGAAGGTTAG | 58.208 | 528 |
| | t2_L | 100880154 | ATGAGGCCGTT | | | 100880681 | CTAATCGAGG | | |
| 2 | 645_e1_ | chr11: | TAGTGTCCA | 58.07 | 645_e1_t1_R | chr11: | ATTGTGGAAG | 58.07 | 525 |
| | t1_L | 100879860 | CAGTAACTAGC | | | 100880384 | CAATTCTCAG | | |
| 3 | 644_e1_ | chr11: | ATTTAGCAC | 58.287 | 644_e1_t0_R | chr11: | GTGAAATCCC | 57.849 | 539 |
| | t0_L | 100867442 | CAACAAGAACC | | | 100867980 | GTCTCTACTA | | |
| 4 | 643_e1_ | chr11: | ACTGAGTAT | 57.886 | 643_e1_t0_R | chr11: | CGTTTATGCTG | 58.093 | 501 |
| | t0_L | 100864599 | CCTTTCACACA | | | 100865099 | AACCTTTCT | | |
| 5 | 642_e1_ | chr11: | CTACCCTGTT | 58.008 | 642_e1_t0_R | chr11: | ATTGGAATGT | 57.97 | 562 |
| | t0_L | 100858644 | GGTTTTCTTC | | | 100859205 | GCAGATGTTT | | |
| 6 | 641_e1_ | chr11: | TGTTGGAAA | 57.988 | 641_e1_t0_R | chr11: | ATAGAACAGC | 58.032 | 408 |
| | t0_L | 100852126 | CTCACAAACAA | | | 100852533 | TAAGGCTGAA | | |
| 7 | 640_e1_ | chr11: | TCCCTCCAA | 58.163 | 640_e1_t0_R | chr11: | TCGCAGAAAA | 58.027 | 497 |
| | t0_L | 100849292 | CTCATTTGTAA | | | 100849788 | AGAAGTTACC | | |
| 8 | 639_e1_ | chr11: | TACTGGTCA | 57.985 | 639_e1_t0_R | chr11: | CAATATTACC | 57.989 | 528 |
| | t0_L | 100847939 | AACGAGTGTAT | | | 100848466 | CCATCCTTGC | | |
| 9 | 638_e1_ | chr11: | TCATGTTGA | 57.948 | 638_e1_t0_R | chr11: | TTTATAGGCAT | 58.01 | 471 |
| | t0_L | 100846957 | ACAGACACAAG | | | 100847427 | TGTCCTCCA | | |
| 10 | 637_e1_ | chr11: | GGTCTGTTCT | 58.064 | 637_e1_t0_R | chr11: | ACATCCTGAA | 57.954 | 593 |
| | t0_L | 100845263 | CTACTTGCTA | | | 100845855 | GATCAACTCA | | |
| 11 | 635_e1_ | chr11: | CTGTCACTT | 58.003 | 635_e1_t0_R | chr11: | TTCTCATGACA | 58.108 | 511 |
| | t0_L | 100830794 | ACAAAATGCCT | | | 100831304 | ACGTGATTG | | |
| 12 | 634_e1_ | chr11: | TCCAAGTCC | 57.93 | 634_e1_t0_R | chr11: | TAGCCTAGGT | 57.951 | 517 |
| | t0_L | 100829353 | ACCATAAGAAT | | | 100829869 | GATGTGAAAA | | |
| 13 | 633_e1_ | chr11: | GGCTTCAAG | 57.995 | 633_e1_t0_R | 10chr11: | CCCACAGTCA | 58.022 | 266 |
| | t0_L | 100828849 | TGGACAAATAA | | | 100829114 | CTAGTTTTTC | | |

The resulting PCR products were subjected to high-throughput sequencing on the Genome Analyzer IIx (Illumina, San Diego, Calif., USA) at the Yale Center for Genomic Analysis. An in-house script was used to generate a list of variants (see Supplementary Materials for more details). Whole-exome data for 10 additional SSC cases were available and filtered for nonsynonymous singleton variants with a SAMtools SNP quality score ≧50. Variant confirmation was performed on blood-derived genomic DNA for the cases, since it was available, and lymphoblastoid cell line-derived genomic DNA for NINDS controls, using conventional PCR and Sanger sequencing. Segregation analysis was performed on blood-derived genomic DNA for cases since family members were available. Chromatograms were aligned and analyzed for variants using the Sequencher v4.9 program (Gene Codes, Ann Arbor, Mich., USA). For the NE controls, whole-exome sequencing data were filtered by the same parameters used for the 10 SSC cases: nonsynonymous singleton variants with a SAM tools SNP quality score ≧50. No read threshold was used to maximize sensitivity over specificity. These variants were not confirmed by Sanger sequencing, but the filtering parameters typically lead to a 70% confirmation rate in our experience. Therefore, we have included the maximum possible number of variants from the NE control cohort. To obtain the exome data, genomic DNA from both the 10 SSC probands and 1930 NE controls had been enriched for exonic sequences using NimbleGen capture and sequenced by the Illumina Genome Analyzer IIX or HiSeq2000. Novelty and singleton status of all variants were determined by comparing all three cohorts and screening dbSNP137 and Exome Variant Server v.0.0.15 (NHLBI GO Exome Sequencing Project (ESP), Seattle, Wash., URL: http://evs.gs.washington.edu/EVS/), accessed Nov. 1, 2012. All p values for mutation burden are two-tailed, calculated from Fisher exact test.

### Supplemental Experimental Procedures

### Mutation Screening of TRPC6

### Multiplex PCR.

Lymphoblastoid cell line-derived genomic DNA was quantitated using PicoGreen dye (Invitrogen, Carlsbad, Calif., USA) on a Synergy HT fluorometer (BioTek, Winooski, Vt., USA). DNAs were then pooled by case/control status, 500 ng/individual, such that all pooled samples were 8 cases or 8 controls for a total of 4 μg input DNA. Pooled samples were sheared on a Covaris S2 to approximately 3 kb (Covaris, Woburn, Mass., USA), then cleaned by Qiagen Min-Elute columns (Qiagen GmbH, Hilden, Germany) with minor modification of the protocol (10 μL 3MNaC₂H₃O₂ was added to the 5:1 PB:sample mix to facilitate proper pH-driven DNA binding and the mix was left on the membrane for 3 minutes before spinning) Samples were dry spun after the PE wash for 2 minutes and eluted with 9.0 μL EB buffer to generate a final volume of 7.74, the input volume of DNA for the RDT1000. Successful shears were determined with the DNA7500 protocol by an Agilent Bioanalyzer 2100 (Agilent Technologies, Santa Clara, Calif., USA).

The sheared genomic DNA pools were combined with RainDance microemulsion PCR master mix prepared according to the protocol. The microemulsion droplet merges were run on the RDT1000 machine (Raindance Technologies, Lexington, Mass., USA). All merges were at least 85% efficient (85% of PCR master mix droplets merged successfully 1:1 with a library primer pair droplet); if not, new DNA pools were sheared and the merge was redone to at least 85% efficiency (considered the threshold for “very good” by RainDance support staff). Successful merges were amplified under the following conditions:

| | | |
|---|---|---|
| Initial denaturation | | 94.0° C., 2 min |
| 55 cycles: | Denaturation | 94.0° C., 30 sec |
| | Annealing | 54.0° C., 30 sec |
| | Extension | 68.0° C., 60 sec |
| Final extension | | 68.0° C., 10 min |

Samples were then purified on Qiagen MinElute columns with 17 μL EB buffer. Eluted product was run on an Agilent Bioanalyzer according to the DNA1000 protocol to determine successful amplification.

PCR product was brought to a volume of 19 μL with the Tris-EDTA buffer. 2.5 μL blunting buffer, 2.5 μL 1 mM dNTPs, and 1 μL blunting enzyme were added (NEB, Ipswich, Mass., USA). This reaction mix was incubated at 22° C. for 15 minutes to blunt, 70° C. for 5 minutes to inactivate the enzyme, and subsequently held at 4° C. Directly after blunting and without cleanup, the PCR products were concatenated into longer DNA fragments; this step was necessary since the range of amplicon sizes in the microemulsion library makes sequencing uniform fragment lengths impossible without first concatenating and then shearing. Concatenation was performed by adding 25 μL NEB Quick Ligase buffer and 5 μL NEB Quick Ligase, mixing thoroughly by pipetting, and transferring to a thermal cycler holding at 22° C. for at least 24 hours. An additional 34 of Quick Ligase was added, the samples were mixed again, and incubated at 37° C. for 1 hour and held at 4° C.

Concatenated samples were sheared on a Covaris S2 to a mean size of ˜200 bp and subsequently processed according to the Illumina multiplexed library preparation protocol. Samples were quantitated on an Agilent Bioanalyzer 2100 (DNA1000 protocol). They were barcoded using Illumina's standard protocol with the barcodes randomly allocated to pools of cases and controls.

### High-Throughput Sequencing.

Two barcoded pools (one of cases, one of controls) were combined in a 1:1 equimolar ratio (quantitated and size-evaluated using the Agilent Bioanalyzer DNA 1000 protocol) and submitted for high-throughput sequencing on a single lane of an Illumina Genome Analyzer IIx (GAIIX). 75 bp single-end reads were generated according to the standard GAIIx protocol; a Phi-X control was run on lane one of each flowcell to ensure accurate and consistent base calls. The data were run through Illumina's Cassava pipeline to generate FASTQ files.

### Alignment and SAMtools Conversion.

Rescaled FASTQ format data were aligned to unmasked human genome build 18 (NCBI 36) using the Burrows-Wheeler Aligner (BWA) with the default settings using the following command: bwa aln -t 8 ‘BWA_reference“Fastq_input’>‘Output.sai’. Aligned reads were converted to SAMtools format using the following command: bwa samse ‘BWA_reference’ ‘Output.sai’ ‘Fastq_input’>‘Output.sam’.

### Trimming Read Ends.

Analysis of the error rates per base pair for each position within the 75 bp read revealed a higher error rate at the start and at the end of the aligned reads than is seen for conventional sequencing. This is likely due to the concatenation and shearing step and reflects reads that cross the boundary between two amplicons. This explanation is also consistent with the low percentage of reads that align to the genome (89% in this experiment compared to 98% seen in whole-exome sequencing). Since the detection of variants is sensitive to variation in error rate, the 75 bp aligned reads were trimmed using an in-house script to remove the first three base pairs and the last eight base pairs in each read resulting in a 64 bp read. The SAM CIGAR string was modified accordingly.

### Filtering to Target.

The aligned reads were filtered to remove reads outside the target amplicons using an in-house script. If any read overlapped at least 1 bp of a target amplicon then the read was considered ‘on-target’. The total target was 501,959 bp of non-overlapping amplicons (not including primers) of which 230,697 bp were regions of interest within the amplicons (Table S6).

### Pileup Conversion.

The filtered aligned data was converted to a sorted binary format (BAM) using SAMtools on the default settings. The following command was used: samtools view -bSt ‘SAM_reference’ ‘Input.sam’|samtools sort−‘Output.bam’. The aligned and filtered SAM file was then converted to pileup format using SAMtools with the default settings: samtools pileup -cAf ‘Reference’ -t ‘SAM_reference’ ‘Input.sam’>‘Output.pileup’.

### Variant Detection.

To determine the optimal thresholds for variant detection within a pool of 8 samples the accuracy of detection was compared with genotyping data (Illumina 1Mv1 BeadArray, 166 SNPs) and Sanger sequencing of the gene PCLO (15,266 bp). The PHRED-like score of bases predicting a variant were seen to follow a bimodal distribution with the data clustered below a score of 10 and above a score of 20. Accordingly, a threshold of 20 was set. The other threshold considered was the frequency of reads representing the variant allele; since the data represented pools of 8 individuals the expected frequency of reads representing a rare heterozygous allele in a single individual was 6.25% (rather than 50% with a single individual). However, due to random variation in genomic DNA, droplet dynamics, and sequencing representation, a substantial proportion of rare heterozygous alleles will have a frequency below the expected threshold. By varying this detection threshold with a PHRED-like score of 20 the optimal detection frequency was determined to be 3.5%. At these thresholds the sensitivity was estimated at 89% for a variant present on a single allele within the pool; the observed positive predictive value was 75% giving an estimate of specificity of 99.9988%. Variants were detected using an in-house script that filtered at these thresholds and was blind to case/control status.

### Variant Annotation and Filtering.

Variants were annotated against the UCSC gene definitions to determine the effect on the resulting amino acid sequence. Where multiple isoforms were present, the most-deleterious interpretation was selected. If the specific variant was present in dbSNPv132 (converted to hg18) the variant was marked. To generate a list of variants of interest for confirmation, variants were filtered to those at allele frequency ≦2% in the dataset and those which are missense, nonsense, or alter the 2 bp splice donor/acceptor sites.

Confirmation of variants was performed by a standard PCR in 25 ul volume:

10 ng genomic DNA

### 1×PCR PreMix D (Epicentre Biotechnologies, Madison, Wis., USA)

0.5 M betaine
0.48 uM forward primer
0.48 uM reverse primer
0.36 ul Taq polymerase (synthesized in-house)
0.072 ul PFU (synthesized in-house)

DNA was amplified in a Tetrad 2 Peltier Thermal Cycler (Bio-Rad Laboratories, Hercules, Calif., USA) using the following cycling parameters:

| | | |
|---|---|---|
| Initial denaturation | | 95.0° C., 5 min |
| 40 cycles: | Denaturation | 95.0° C., 30 sec |
| | Annealing | 54.0° C., 30 sec |
| | Extension | 72.0° C., 60 sec |
| Final extension | | 72.0° C., 10 min |

PCR products were visualized by agarose gel electrophoresis and sent to the Yale Keck Biotechnology Resource Laboratory for Sanger sequencing. Chromatograms were aligned and analyzed for variants using the Sequencher v4.9 program (Gene Codes, Ann Arbor, Mich., USA).

### Case/Control Population Matching and Quality Control Single Nucleotide Polymorphism (SNP) Genotyping:

Blood-derived genomic DNA was used from SSC cases; only lymphoblastoid cell line-derived genomic DNA was readily available and used from NINDS controls. SSC cases (n=1195) were genotyped using the IlluminaHuman1M-Duo v1, Human 1M-Duo v3, or HumanOmni2.5 BeadChips, according to the standard Illumina protocol. NINDS controls (n=953) were genotyped using the Illumina HumanOmniExpress12v1. All genotyping was performed at the Yale Keck Biotechnology Resource Laboratory.

### SNP Quality Control:

Sample genotypes were analyzed using PLINK (Purcell et al., 2007) and removed from the analyses if: 1) sample call rate was less than 95%, 2) genotypes were inconsistent with recorded gender, or 3) Mendelian inconsistencies or cryptic relatedness were detected by assessing inheritance by descent (IBD). The following PLINK commands were used:
plink --bfile<Samplefile>--check-sex
plink --bfile<Samplefile>--mendel
plink --bfile<Samplefile>--extract <Hapmap_LD.prune.in >--mind 0.05-geno 0.1 --maf 0.01 --hwe-all --make-bed --out <Samplefile.indep>
plink --bfile<Samplefile.indep>--genome --min 0.05 --out <Sample.IBD.Result>

‘Hapmap_LD.prune.in’ is a pre-defined list of 129,932 independent SNPs to ensure consistency of results across samples of different sizes. This SNP list was derived from 120 Hapmap individuals with 1Mv1 Illumina data using the command:
plink --bfileHapmapfileindep-pairwise 50 5 0.2 --out Hapmap_LD.prune.in No instances of cryptic relatedness were detected.

### Population Outlier Exclusions:

Golden Helix SNP and Variation Suite v7.5.4 (Golden Helix, Bozeman, Mont., USA) was used to perform a genotype principal component analysis (PCA) of the SSC cases and NINDS controls, using 8117 consensus SNPs common to all array platforms and not found to be in high linkage disequilibrium. Based on visualization of a screen plot (FIG. 10A), Eigenvalues of the first three principal components were plotted against one another (FIG. 10B), and the interquartile range (IQR) distance around the median of the study population cluster was calculated. A threshold that included all NINDS controls was determined to lie at 6 IQRs from the third quartile, and SSC cases beyond this threshold were excluded as ancestral outliers (FIG. 10C) for calculation of mutation burden. PCA of genotyping data using Eigenstrat of the 1930 neuropsychiatrically unscreened NE controls used in the omnibus analysis revealed that these controls cluster tightly with the HapMap CEU cohort.

### Results

Characterization of the t(3;11)(p21;q22) Translocation Breakpoint

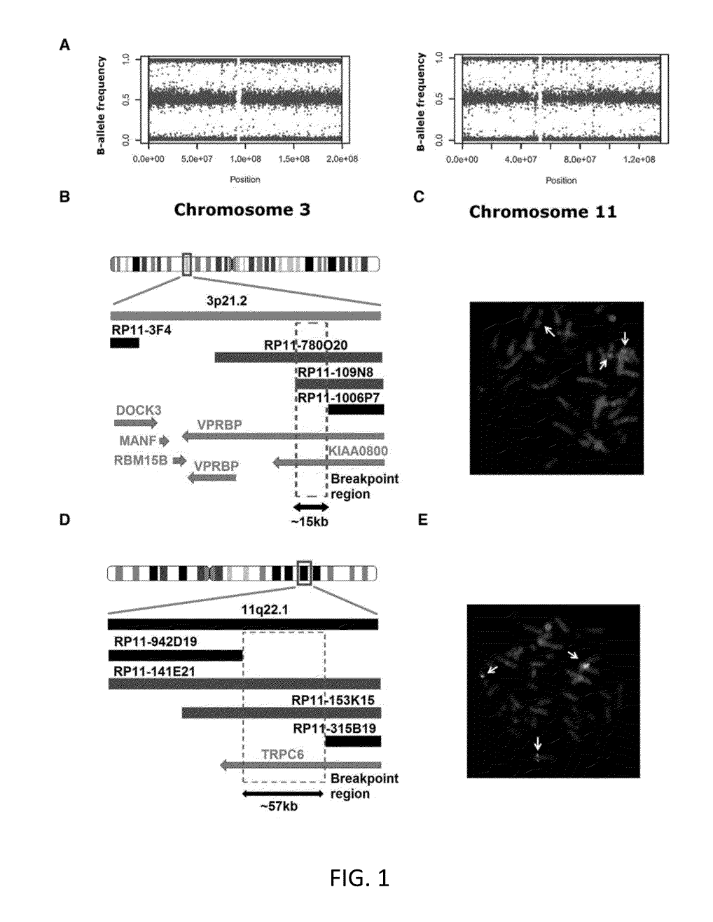

We ascertained an eight year old male autistic patient who was found to carry a de novo 46, XY, t(3;11)(p21;q22) translocation by G-banding karyotyping of lymphoblastoid cells. No gain or loss of genetic material was found near the breakpoint areas via genome wide array analysis (FIG. 1A). Only a duplication (104.225.150 bp-104.339.273 bp) on chromosome 14 was identified, which was previously shown to be a common copy number variant (CNV; http://projects.tcag.ca/variation/). Fluorescent In Situ Hybridization (FISH) analysis revealed that BAC probes RP11-780O20 and RP11-109N8 span the breakpoint on chromosome 3p21, while probes RP11-3F4 and RP11-1006P7 map distal and proximal to the breakpoint, respectively (FIG. 1B, C). This narrowed the breakpoint to an interval of approximately 15 Kb spanning the gene encoding the Vpr binding protein (VPRBP), indicating that this gene was disrupted. Similarly, the breakpoint on chromosome 11q22 was mapped to a region spanned by probes RP11-141E21 and RP11-153K15, distal to RP11-315B9 and proximal to RP11-942D19 (FIG. 1D, E), suggesting disruption of the TRPC6 gene, which was confirmed by the use of additional strategies. We first measured the expression levels of TRPC6 exons 4, 6, 12 and 13 in the lymphocytes of the patient, his parents and six control individuals by quantitative real-time PCR (qPCR) (FIG. 6A). In the patient's parents and in controls, exons 6, 12 and 13 have similar expression levels as exon 4. In the patient, however, the expression levels of exons 12 and 13 were reduced by approximately 50% when compared to exon 4. After sequencing all TRPC6 exons, we found that the patient is heterozygous for two common polymorphisms: one mapping to exon 6 (rs12366144) and the other to exon 13 (rs12805398). However, we identified heterozygosity only for the polymorphism in exon 6 when cDNA from the patient's lymphocytes was sequenced (FIG. 6B). Parentage was confirmed by genotyping of microsatellite markers (FIG. 6C). These results show that TRPC6 has biallelic expression and that heterozygosity loss in exon 13 in the patient's cDNA can be explained by the disruption of TRPC6. Accordingly, TRPC6 is transcribed up to the breakpoint, which should be located between exons 6 and 12. We did not identify any pathogenic change in TRPC6 exons upon sequencing patient's DNA.

Disruption of TRPC6, VPRBP and several other unknown genes, could be contributing to the phenotype in this patient. TRPC6 is a Ca²⁺-permeable nonselective cation channel involved in neuronal survival, growth cone guidance and spine and synapse formation, biological processes already implicated in ASD etiology (Jia et al., 2007; Li et al., 2005; Tai et al., 2009; Tai et al., 2008; Zhou et al., 2008). The function of VPRBP (Vpr binding protein) is less clear and may include DNA replication, S-phase progression and cellular proliferation (McCall et al., 2008). As functional analyses are time consuming, we elected to focus on additional genetic and functional studies of TRPC6, not previously associated with ASDs.

### Disruption of TRPC6 Leads to Transcriptional Alterations and Dysregulation of CREB Phosphorylation

To determine whether disruption of one TRPC6 allele leads to dysregulation of gene transcription due to perturbations of the signaling pathway activated by this channel, we conducted a global expression analysis comparing the patient's dental pulp stem cells (DPSCs) to 6 control samples. DPSCs, easily isolated from the deciduous teeth of ASD patients in a non-invasive procedure (Gronthos et al., 2002), have an ectodermic neural crest origin and express several neuronal markers (d'Aquino et al., 2009; Miura et al., 2003). We identified 67 differentially expressed genes (DEGs) between the TRPC6-mutant patient and non-affected controls (p<0.05; Table S1).

TABLE S1
Differentially expressed genes between TRPC6-mutant patient and controls.

| | | | Regulation by |
|---|---|---|---|
| Gene | Fold change* | Name | CREB** |
| RGS4 | −3.424461441 | regulator of G-protein signaling 4 | In silico |
| PRRX1 | −2.374475207 | paired related homeobox 1 | In silico; arrays |
| PTGS2 | −3.49316255 | prostaglandin-endoperoxide synthase 2 | ChIP-on-chip, |
| | | (prostaglandin G/H synthase and | Gosh et al., 2007 |
| | | cyclooxygenase) | |
| INA | −2.639988194 | internexin neuronal intermediate | ChIP-on-chip |
| | | filament protein, alpha | |
| ITGA7 | −3.173058661 | integrin, alpha 7 | In silico |
| NPTX1 | −2.855578291 | neuronal pentraxin I | In silico |
| ANGPTL4 | −3.15182498 | angiopoietin-like 4 | In silico |
| MAP2 | −2.789671289 | microtubule-associated protein 2 | ChIP-on-chip |
| EPHA4 | 2.362428255 | EPH receptor A4 | ChIP-on-chip; |
| | | | In silico |
| PMEPA1 | −3.901062426 | prostate transmembrane protein, | In silico |
| | | androgen induced 1 | |
| CLDN11 | 4.066602785 | claudin 11 | In silico, |
| | | | Lui et al., 2007 |
| PCDH10 | −4.318180517 | protocadherin 10 | ChIP-on-chip; |
| | | | In silico |
| ESM1 | −5.824585313 | endothelial cell-specific molecule 1 | In silico |
| DSP | −3.879025353 | desmoplakin | ChIP-on-chip |
| FLNC | −2.328384225 | filamin C, gamma | in silico |
| CLDN1 | 4.171417178 | claudin 1 | In silico |
| SEMA3A | 2.314408538 | semaphorin 3A | — |
| CDH6 | −2.67546301 | cadherin 6, type 2, K-cadherin (fetal | — |
| | | kidney) | |
| CFH | 3.295805 | complement factor H | — |
| CTSK | 2.538857806 | cathepsin K | — |
| TNFSF18 | 2.364541617 | tumor necrosis factor (ligand) | — |
| | | superfamily, member 18 | |
| IFIT1 | 3.0693729 | interferon-induced protein with | — |
| | | tetratricopeptide repeats 1 | |
| IGF2 | 2.643577319 | insulin-like growth factor 2 | — |
| | | (somatomedin A) | |
| CASP1 | 2.545250054 | caspase 1, apoptosis-related cysteine | — |
| | | peptidase (interleukin 1, beta, | |
| | | convertase) | |
| CRYAB | −2.865558657 | crystallin, alpha B | — |
| PDE3A | −3.350362985 | phosphodiesterase 3A, cGMP- | — |
| | | inhibited | |
| LUM | 2.412407948 | lumican | — |
| EPSTI1 | 3.159449161 | epithelial stromal interaction 1 (breast) | — |
| LTBP2 | −2.717385789 | latent transforming growth factor beta | — |
| | | binding protein 2 | |
| VCAM1 | 4.546975557 | vascular cell adhesion molecule 1 | — |
| ACAN | −4.199956627 | aggrecan | |
| CCL2 | 2.41655874 | chemokine (C-C motif) ligand 2 | — |
| RAB27B | 2.89104252 | RAB27B, member RAS oncogen | — |
| | | family | |
| KYNU | 3.411663783 | kynureninase (L-kynurenine | — |
| | | hydrolase) | |
| EFEMP1 | 2.641250593 | EGF-containing fibulin-like | — |
| | | extracellular matrix protein 1 | |
| MX2 | 2.490495298 | myxovirus (influenza virus) | — |
| | | resistance 2 (mouse) | |
| MX1 | 2.63120465 | myxovirus (influenza virus) | — |
| | | resistance 1, interferon-inducible | |
| | | protein p78 (mouse) | |
| MME | 3.182582502 | membrane metallo-endopeptidase | — |
| PTX3 | 3.998647561 | pentraxin-related gene, rapidly | — |
| | | induced by IL-1 beta | |
| PCOLCE2 | −3.094230756 | procollagen C-endopeptidase | — |
| | | enhancer 2 | |
| TLR3 | 3.212189239 | toll-like receptor 3 | — |
| SULT1B1 | −4.384388323 | sulfotransferase family, cytosolic, 1B, | — |
| | | member 1 | |
| PLAC8 | 2.728492911 | placenta-specific 8 | — |
| ASB5 | 2.862047519 | ankyrin repeat and SOCS box- | — |
| | | containing 5 | |
| AKAP12 | −4.129093856 | A kinase (PRKA) anchor protein 12 | — |
| AEBP1 | −2.359467987 | AE binding protein 1 | — |
| LRRC17 | 2.310487698 | leucine rich repeat containing 17 | — |
| CPA4 | 2.497337774 | carboxypeptidase A4 | — |
| HGF | 4.252390982 | hepatocyte growth factor | |
| | | (hepapoietin A; scatter factor) | — |
| TFPI2 | −2.967261549 | tissue factor pathway inhibitor 2 | — |
| PODXL | −3.832320219 | podocalyxin-like | — |
| PTGS1 | −3.737081399 | prostaglandin-endoperoxide synthase 1 | — |
| | | (prostaglandin G/H synthase and | |
| | | cyclooxygenase) | |
| IFI44L | 3.938933542 | interferon-induced protein 44-like | — |
| OLFML3 | 2.585096521 | olfactomedin-like 3 | — |
| C11orf 41 | −2.524039553 | chromosome 11 open reading frame 41 | — |
| C11orf41 | −2.756412512 | chromosome 11 open reading frame 41 | — |
| FAM111B | −2.387132177 | family with sequence similarity 111, | — |
| | | member B | |
| Cl3orf15 | 2.878825964 | chromosome 13 open reading frame 15 | — |
| MYOCD | −3.398938999 | myocardin | — |
| SLFN11 | −3.488715196 | schlafen family member 11 | — |
| SNORD35A | −2.273343207 | small nucleolar RNA, C/D box 35A | — |
| ABI3BP | −3.714334634 | ABI family, member 3 (NESH) | — |
| | | binding protein | |
| PCDH18 | 2.508559732 | protocadherin 18 | — |
| C4orf49 | 3.302012711 | chromosome 4 open reading frame 49 | — |
| C9orf150 | 3.202042462 | chromosome 9 open reading frame 150 | — |
| LOC554202 | −2.481918054 | hypothetical LOC554202 | — |
| C9orf150 | 3.202042462 | chromosome 9 open reading frame 150 | — |

*Logarithmic gene expression difference between TRPC6 disrupted patient and controls
**Evidences of gene transcription regulation by the transcription factor CREB according to the database http://natural.salk.edu/CREB/search.htm: Zhang and colleagues (2005) used three different strategies to identify the genes regulated by CREB: in silico analysis, chromatin co-immunopreciptation followed by microarray analysis (ChIP-on-chip) and expression analysis of genes induced by forskolin (array). The genes for which no evidence of CREB regulation was found are annotated as “—”; those for which no information is available in the adopted database are annotated as “—”.

Functional annotation analysis revealed that 16 of them (24%) have a role in nervous system development and function (Table 1). Using the CREB-target genes database (http://natural.salk.edu/CREB/), we found eight out of these 16 functionally relevant DEGs that are regulated by CREB, a transcription factor activated upon Ca²⁺ influx through TRPC6 (Tai et al., 2008). Among the functionally relevant DEGs, we selected six CREB-target genes (INA, MAP2, NPTX1, CLDN11, PCDH10 and EPHA4) and two other genes (SEMA3A and CDH6) to be evaluated by quantitative PCR (qPCR) as validation for the microarray experiments. The expression of CDH6, INA, MAP2 and CLDN11 in the TRPC6-mutant patient is dysregulated in comparison to the controls in the same direction as observed in the microarray analysis (p<0.05; Table 1).

TABLE 1
Selected Functionally relevant genes differentially expressed between
TRPC6-mutant patient and controls.

| | | | | qPCR |
|---|---|---|---|---|
| | | | Regulation by | validation |
| Gene | Fold change* | Gene Ontology | CREB** | (P value) |
| INA | −2.639988194 | nervous system | ChIP-on-chip | 0.0198 |
| | | development; neurofilament | | |
| | | cytoskeleton organization | | |
| NPTX1 | −2.855578291 | growth of neurites; synaptic | In silico | 0.0885 |
| | | transmission; central | | |
| | | nervous system development | | |
| MAP2 | 2.789671289 | growth of neurites; | ChIP-on-chip | 0.0363 |
| | | development and elongation | | |
| | | of neurites; patterning of | | |
| | | cerebral cortex; polarization | | |
| | | of hippocampal neurons | | |
| EPHA4 | 2.362428255 | guidance of axons; | ChIP on chip; | 0.4305 |
| | | formation of pyramidal | In silico | |
| | | tract; axon guidance | | |
| CLDN11 | 4.066602785 | axon ensheathment; | In silico | 0.0005 |
| | | calcium-independent cell- | Lui et al., | |
| | | cell adhesion; migration of | 2007 | |
| | | neuroglia | | |
| PCDH10 | −4.318180517 | cell adhesion; establishment | ChIp-on-chip; | 0.3331 |
| | | and function of specific cell- | In silico | |
| | | cell connections in the brain | | |
| CLDN1 | 4.171417178 | calcium independent cell- | In silico | |
| | | cell adhesion; myelination | | |
| | | of cells | | |
| PTGS2 | −3.49316255 | activation of astrocytes; | ChIp-on-chip | |
| | | activation of neuroglia; | Gosh et al., | |
| | | memory; positive regulation | 2007 | |
| | | of synaptic plasticity; | | |
| | | negative regulation of | | |
| | | synaptic transmission, | | |
| | | dopaminergic; positive | | |
| | | regulation of synaptic | | |
| | | transmission, glutamatergic | | |
| CDH6 | −2.675463010 | cell-adhesion; establishment | — | 0.0418 |
| | | and function of specific cell- | | |
| | | cell connections in the brain | | |
| SEMA3A | 2.314408538 | nervous system | — | 0.1828 |
| | | development; axonal | | |
| | | fasciculation; regulation of | | |
| | | axon extension involved in | | |
| | | axon guidance; distribution | | |
| | | of neurons; migration of | | |
| | | neuroglia; growth of | | |
| | | neurites; chemorepulsion of | | |
| | | sympathetic neuron | | |
| CASP1 | 2.545250054 | activation of astrocytes | — | |
| | | activation of neuroglia | | |
| VCAM1 | 4.546975557 | growth of neurites; | | |
| | | distribution of neurons; cell | | |
| | | adhesion; guidance of axons | | |
| ACAN | −4.199956627 | growth of neurites; cell | — | |
| | | adhesion | | |
| CCL2 | 2.41655874 | cell adhesion; astrocyte cell | — | |
| | | migration | | |
| HGF | 4.252390982 | growth of neurites; | — | |
| | | complexity of dendritic trees | | |
| PCDH18 | 2.508559732 | cell adhesion; brain | — | |
| | | development | | |

*Logarithmic gene expression difference between patient and controls
**Evidence of gene transcription regulation by the transcription factor CREB according to the database http://natural.salk.edu/CREB/search.htm: Zhang and colleagues (2005) used three different strategies to identify the genes regulated by CREB: in silico analysis, chromatin co-immunopreciptation followed by microarray analysis (ChIP-on-ChIP) and expression analysis of genes induced by forskolin (array). The genes for which no evidence of CREB regulation was found are annotated as “—”; those for which no information is available in the adopted database are annotated as “—”.

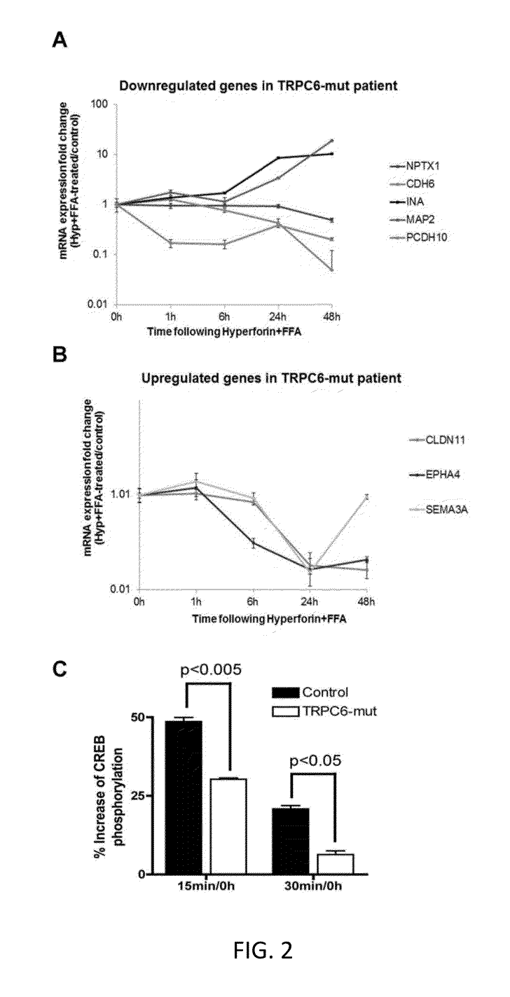

To further validate our hypothesis that TRPC6 haploinsufficiency leads to transcription dysregulation, we treated a control DPSC culture with hyperforin plus flufenamic acid (FFA), and measured the expression levels of the same candidate genes across 48 hours (FIG. 2A, B). Hyperforin specifically activates TRPC6, and FFA increases the amplitude of the currents through this channel (Leuner et al., 2010; Leuner et al., 2007; Muller et al., 2008). If the candidate genes are regulated through TRPC6, we would expect a change in their expression levels in the opposite direction to that found in the TRPC6-mutant patient. After a 48-hour treatment, we found the expected correlation for five out of the eight genes. While SEMA3A, EPHA4 and CLDN11 had their expression significantly reduced, MAP2 and INA showed a 20- and 10-fold increase in expression respectively. Therefore, these results further validate the microarray data and support the hypothesis that the selected genes are upon regulation of TRPC6 pathway.

We also measured CREB phosphorylation in the DPSCs of this patient and a control to test the functional effect of TRPC6 disruption. The stimulation of DPSCs with hyperforin plus FFA showed that the control sample had an increase in the levels of phosphorylated CREB (p-CREB) of 48% and 30%, after 15 and 30 minutes respectively. During the same time intervals, the levels of p-CREB in the patient's cells increased only 20% and 6%, a percentage significantly reduced compared to the control level (FIG. 2C). Taken together, these results show that several of the functionally relevant DEGs identified in the microarray studies are controlled via TRPC6 signaling, likely through CREB phosphorylation, suggesting that disruption of TRPC6 influences neuronal cell function.

Generation of Neuronal Cells from ASD Patients

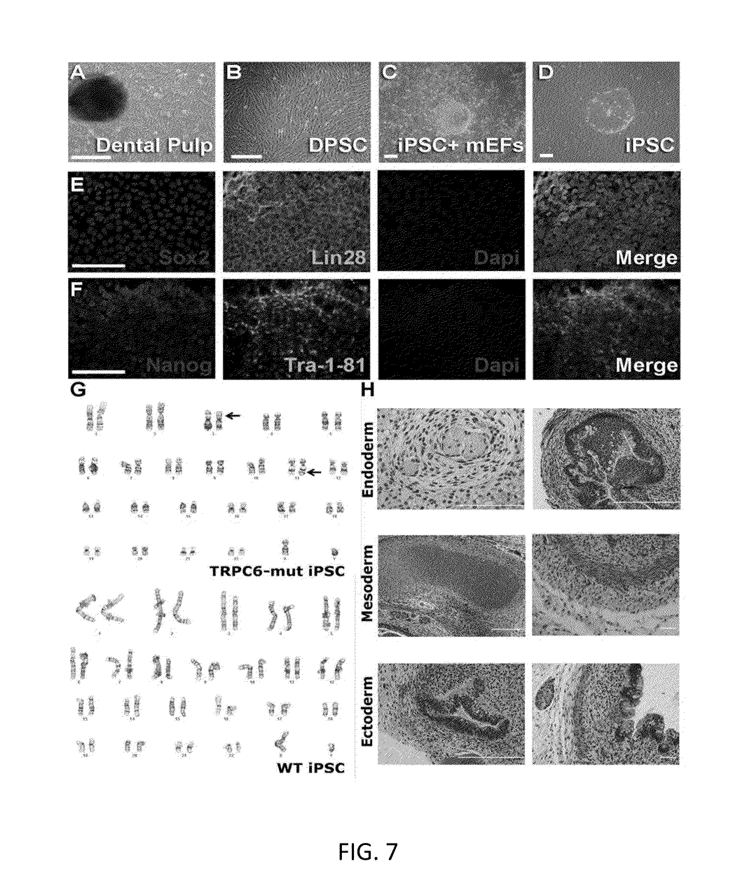

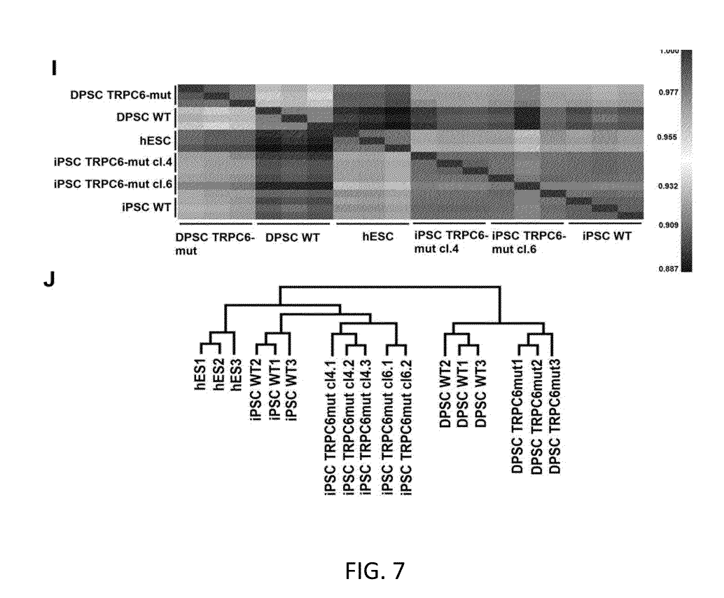

To further evaluate the effect of TRPC6 haploinsufficiency in the function of neural cells, we generated iPSCs from the patient and two control individuals (FIG. 7 and Table S2).

TABLE S2
Fingerprinting analysis of DPSC and iPSC lineages from TRPC6-disrupted
patient and control sample.

| | Marker | | | | | | | | | | | |
|---|---|---|---|---|---|---|---|---|---|---|---|---|
| | | | | | Idiopathic | | Idiopathic | | WT | | | |
| | TRPC6mut | | TRPC6mut | | patient | | patient | | DPSC | | WT iPSC | |
| | DPSC | | iPSC | | DPSC | | iPSC | | (USC1) | | (USC1) | |
| Amelogenin | X | Y | X | Y | X | Y | X | Y | X | Y | X | Y |
| vWA | 17 | 18 | 17 | 18 | 17 | 18 | 17 | 18 | 16 | 18 | 16 | 18 |
| D8S1179 | 11 | 15 | 11 | 15 | 13 | 15 | 13 | 15 | 13 | 15 | 13 | 15 |
| TPOX | 8 | 11 | 8 | 11 | 8 | 8 | 8 | 8 | 9 | 12 | 9 | 12 |
| FGA | 19 | 23 | 19 | 23 | 20 | 22 | 20 | 22 | 20 | 23 | 20 | 23 |
| D3S1358 | 15 | 15 | 15 | 15 | 16 | 16 | 16 | 16 | 16 | 16 | 16 | 16 |
| THO1 | 6 | 9.3 | 6 | 9.3 | 7 | 9 | 7 | 9 | 9 | 9 | 9 | 9 |
| D21S11 | 31 | 31.2 | 31 | 31.2 | 30 | 31.2 | 30 | 31.2 | 30.2 | 32 | 30.2 | 32 |
| D18S51 | 10 | 14 | 10 | 14 | 12 | 12 | 12 | 12 | 19 | 19 | 19 | 19 |
| Penta E | 5 | 11 | 5 | 11 | 9 | 11 | 9 | 11 | 11 | 12 | 11 | 12 |
| D5S818 | 13 | 13 | 13 | 13 | 12 | 12 | 12 | 12 | 12 | 12 | 12 | 12 |
| D13S317 | 10 | 14 | 10 | 14 | 9 | 11 | 9 | 11 | 8 | 13 | 8 | 13 |
| D7S820 | 12 | 13 | 12 | 13 | 10 | 12 | 10 | 12 | 11 | 11 | 11 | 11 |
| D16S539 | 9 | 11 | 9 | 11 | 12 | 12 | 12 | 12 | 12 | 13 | 12 | 13 |
| CSF1PO | 10 | 11 | 10 | 11 | 10 | 11 | 10 | 11 | 11 | 11 | 11 | 11 |
| Penta D | 9 | 9 | 9 | 9 | 12 | 12 | 12 | 12 | 9 | 11 | 9 | 11 |

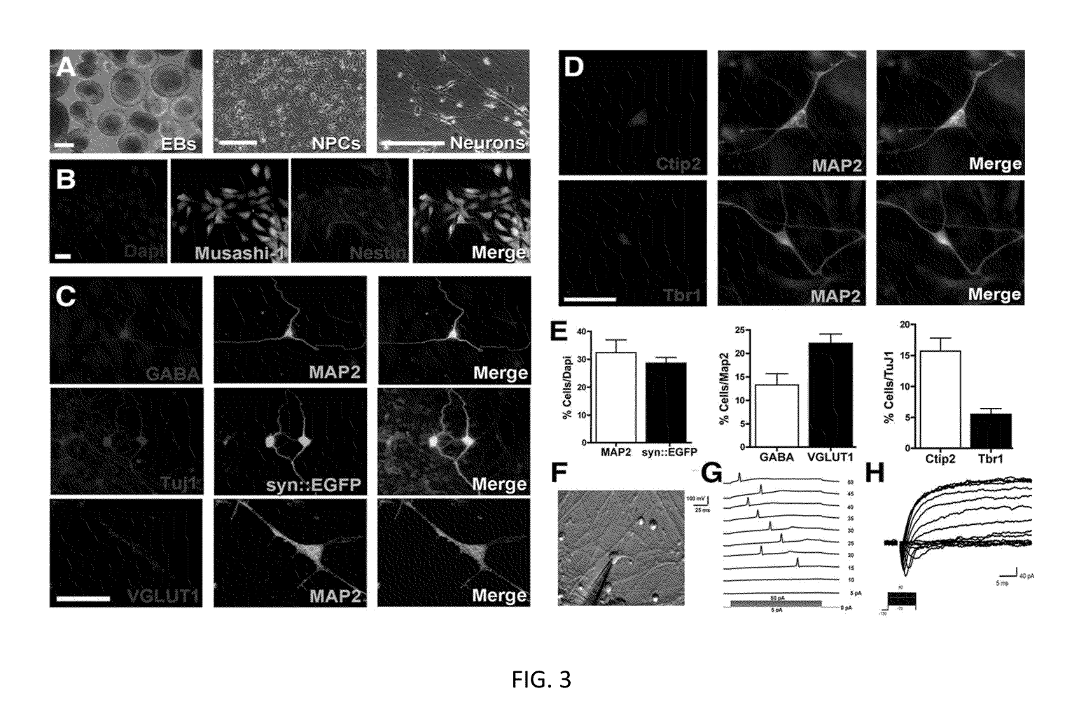

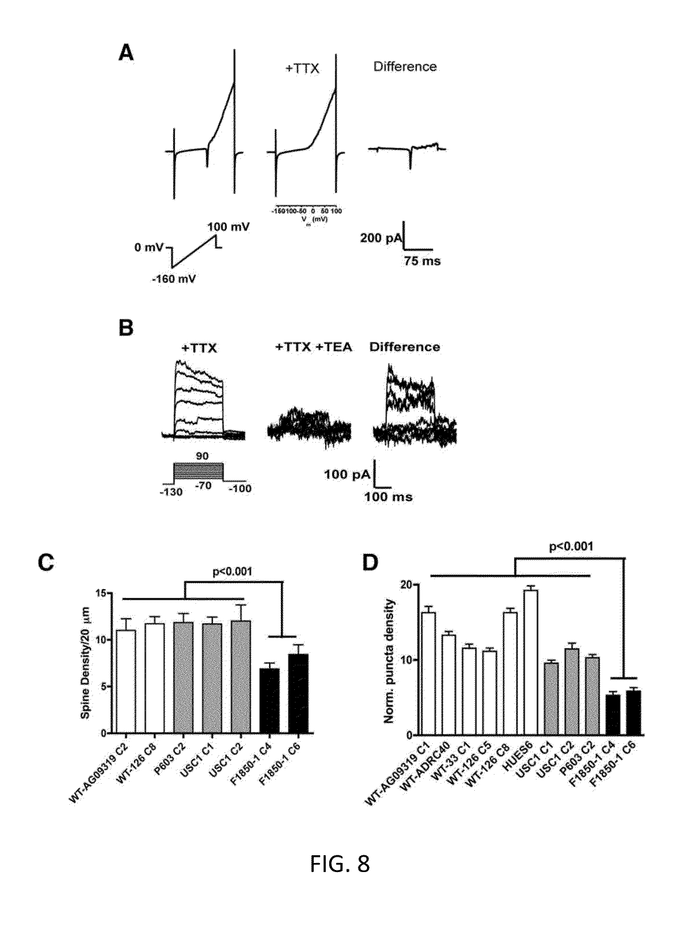

We fully characterized three clones from each individual and used at least 2 different clones for follow up experiments. A summary of the clones used for each experiment can be found in Table S3. Neural progenitor cells (NPCs) and cortical neurons from iPSCs were obtained using a modified protocol from our previous publication (Marchetto et al., 2010). Briefly, iPSC colonies on matrigel were treated with dorsomorphin under FGF-free conditions until confluence. Slices of iPSC colonies were grown in suspension for 2-3 weeks as embryoid bodies (EBs) in the presence of dorsomorphin (FIG. 3A). After this period, EBs were dissociated and plated to form rosettes. The rosettes were manually selected and expanded as NPCs (FIG. 3A). These NPCs were positive for early neural-specific markers, such as Musashi-1 and Nestin (FIG. 3B). To obtain mature neurons, NPCs were plated with ROCK inhibitor and maintained for 3-4 more weeks in differentiation conditions. At this stage, cells were positive for the pan neuronal marker Tuj1 (β-III-Tubulin) and expressed the more mature neuronal markers synapsin and MAP2 (Microtubule-associated protein 2) (FIG. 3C). We also detected expression of the inhibitory neurotransmitters GABA (γ-aminobutyric acid) in about 10% of the neurons and 20% were positive for VGLUT1 (vesicular glutamate transporter-1), a marker for excitatory neurons, in both controls and ASD subjects (FIG. 3C). Our protocol generated a consistent population of forebrain neurons, confirmed by the co-localization of pan-neuronal and subtype specific cortical markers, such as 15% of Ctip2 (Layers VI and V) and 5% of Tbr1 (Layers I and IV) (FIG. 3D-E). Expression of peripherin and En1, markers for peripheral and midbrain neurons, respectively, were not detected. We did not observe a significant variability in these subtypes of neurons between controls and ASD backgrounds. Next, we determined the functional maturation of the iPSC-derived neurons using electrophysiological methods. Whole-cell recordings were performed from cells that had differentiated for at least 6 weeks in culture. Both controls and ASD-neurons showed action potentials evoked by somatic current injections (FIG. 3F-H and FIG. 8A, B). Therefore, our data indicated that somatic cell reprogramming did not affect the ability of iPSC-derived neurons to mature and become electrophysiologically active.

### Ca²⁺ Influx is Reduced in TRPC6-Mutant Patient NPCs

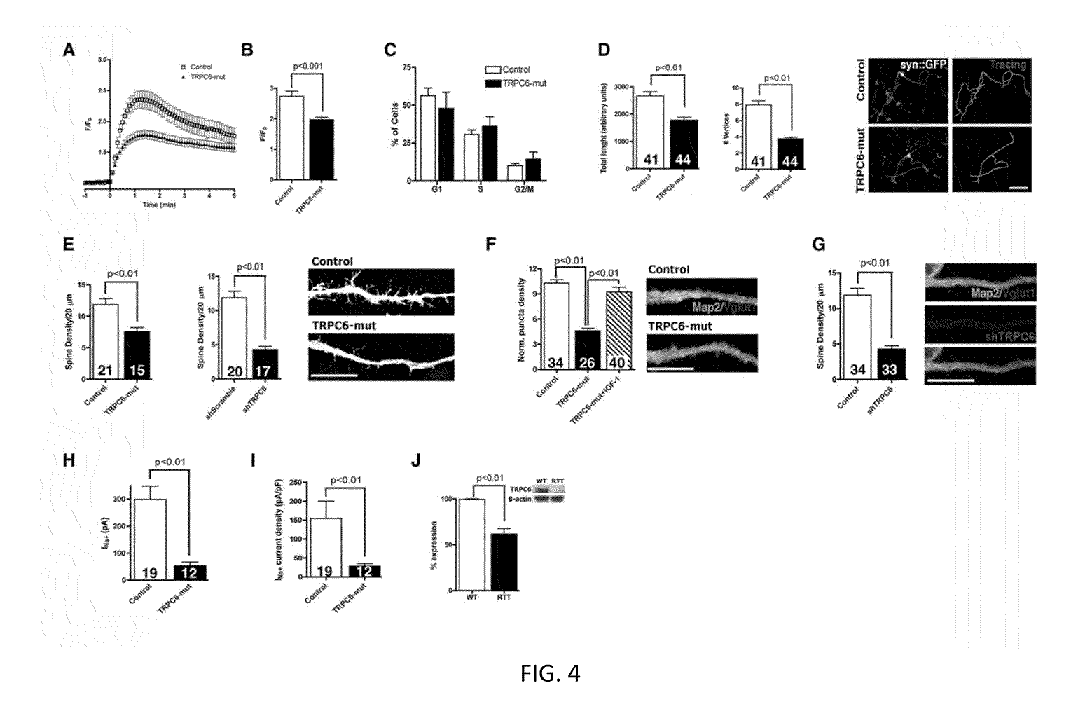

The role of TRPC6 in dendritic spine formation depends on a pathway that involves Ca²⁺ influx through the channel (Tai et al., 2008). Accordingly, it is reasonable to propose that changes in intracellular Ca²⁺ levels may be altered in patient's neural cells. To test this hypothesis, we stimulated iPSC-derived NPCs from the patient and a control with hyperforin plus FFA. This combination of drugs induced transient and repetitive increases in intracellular Ca²⁺ concentration in both patient- and control-derived NPCs. The peak of TRPC6 activation-induced Ca²⁺ oscillations was significantly higher in control NPCs compared to patient NPC (FIG. 4A). The average amplitude of Ca²⁺ increases in the approximately 100 cells analyzed was reduced by about 40% in the patient's NPCs compared to the control sample when stimulated by hyperforin and FFA (p<0.001; FIG. 4B).

TRPC6 Disruption does not Affect NPCs Proliferation

TRPC1, another member of the transient receptor potential channel family, is involved in NPC proliferation mediated by FGF (Fiorio Pla et al., 2005). Therefore, we investigated if reduction of TRPC6 expression levels would affect the cell cycle profile. When we compared the TRPC6-mutant patient iPSC-derived NPCs to control NPCs, we did not identify any difference, indicating that TRPC6 probably does not play a role in NPC proliferation as does TRPC1 (FIG. 4C).

### TRPC6 Disruption Alters Neuronal Phenotype

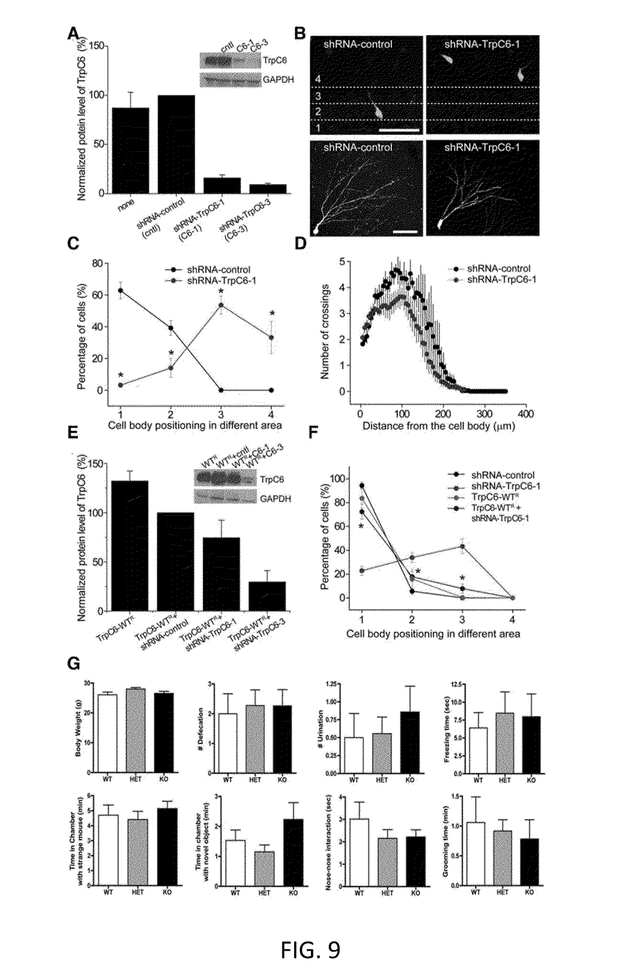

To determine whether TRPC6 disruption influences spine formation and synaptogenesis, we investigated neurons derived from patient's and control's iPSCs. To avoid variability from reprogramming, all experiments were performed with different iPSC clones and independent experiments. We summarize all the biological replicates and iPSC clones used in each experiment in Table S3. We first examined the morphology of the neurons by infecting cells with a previously described lentiviral vector containing the EGFP sequence under the control of the synapsin gene promoter (syn::EGFP) (Marchetto et al., 2010). By measuring the size of neurites and their ramifications, we verified that the TRPC6-mutant patient's neurons are shorter and less arborized compared to controls (FIG. 4D). Moreover, the density of dendritic spines in TRPC6-mutant neurons was reduced compared to control neurons derived from several individuals (FIG. 4E, FIG. 8C). TRPC6 expression was previously demonstrated to regulate spine density (Tai et al., 2008). Thus, to confirm that the alterations observed in TRPC6-mutant neurons was caused by loss of function, we downregulated TRPC6 expression in control neurons using a specific shRNA in a lentiviral vector. We used previously validated shRNAs and re-confirmed the efficiency and specificity in co-transfections in HEK293 cells (FIG. 9A). Neurons derived from control iPSCs expressing the shTRPC6 showed a significant reduction in spine density when compared to control neurons expressing a scrambled shRNA (shScramble) (FIG. 4E).

TRPC6 is mainly expressed in glutamatergic synapses and interferes with synapsin-1 cluster density in pre-synaptic sites of hippocampal neurons, suggesting that this gene has an important role in the regulation of excitatory synapse strength (Zhou et al., 2008). In fact, counting the number of VGLUT1 puncta in MAP2-labeled neurons, we verified that the TRPC6-mutant patient's neurons have a significantly lower density of VGLUT1 puncta compared to independent clones isolate from several controls (FIG. 4F, FIG. 8D). Control neurons expressing shTRPC6 also presented a lower density of VGLUT1 puncta, indicating that loss of TRPC6 function can affect the formation of glutamatergic synapses (FIG. 4G). Finally, during our electrophysiological recordings, we noticed that TRPC6-mutant neurons have impaired Na⁺ currents compared to controls (FIG. 4H, I). Decreased Na⁺ current densities was previously reported in other ASD models (Han et al., 2012).

### TRPC6 and MeCP2 Share the Same Molecular Pathway

Certain neuronal phenotypes (reduction of spine density and glutamatergic synapses) associated with loss of TRPC6 function are similar to those previously described for loss of MeCP2 function in human neurons (Marchetto et al., 2010). MeCP2 genetic alterations have been recognized in several idiopathic ASD patients (Campos et al., 2011; Carney et al., 2003; Cukier et al., 2012; Cukier et al., 2010; Dotti et al., 2002; Kuwano et al., 2011; Lam et al., 2000; Piton et al., 2011) and reduced MeCP2 expression was previously reported in autistic brains (Nagarajan et al., 2006; Samaco et al., 2004). Additionally, two independent articles previously reported that MeCP2 regulates TRPC6 expression in the mouse brain, likely by an indirect mechanism (Chahrour et al., 2008; Li et al., 2012). Thus, we decided to investigate whether MeCP2 is acting upstream of TRPC6 in human neurons. We used two iPSC clones from a female RTT patient carrying the T158M MeCP2 mutation that have persistent X chromosome inactivation (Marchetto et al., 2010). Each clone expresses a different MeCP2 allele, a wild type or mutant version of the MeCP2 gene. We then differentiated both clones into neurons and evaluated the expression of TRPC6 protein levels. The clone that carries the non-functional MeCP2 version has approximately half of the TRPC6 expression compared to the wild type control clone, indicating that MeCP2 regulates TRPC6 expression in human neurons (FIG. 4I). This observation supports the idea that MeCP2 is acting upstream of TRPC6 in the same molecular pathway, affecting neuronal morphology and synapse formation. Our data suggest that the molecular pathway involving MeCP2 and TRPC6 is a rate-limiting factor in regulating glutamatergic synapse number in human neurons. Administration of insulin growth factor-1 (IGF-1) promotes reversal of the RTT-like symptoms in a mouse model (Tropea et al., 2009) and in human neurons (Marchetto et al., 2010) and is currently in clinical trials for RTT patients. To test if the convergence of molecular mechanisms underlying RTT and idiopathic autism could have future therapeutical benefits, we treated TRPC6-mutant neurons with IGF-1 (10 ηg/mL) for two weeks. After that, we observed an increase in glutamatergic synapse number, suggesting that the drug treatment could correct the neuronal phenotype (FIG. 4F).

### TRPC6 Downregulation Compromises Neuronal Development In Vivo

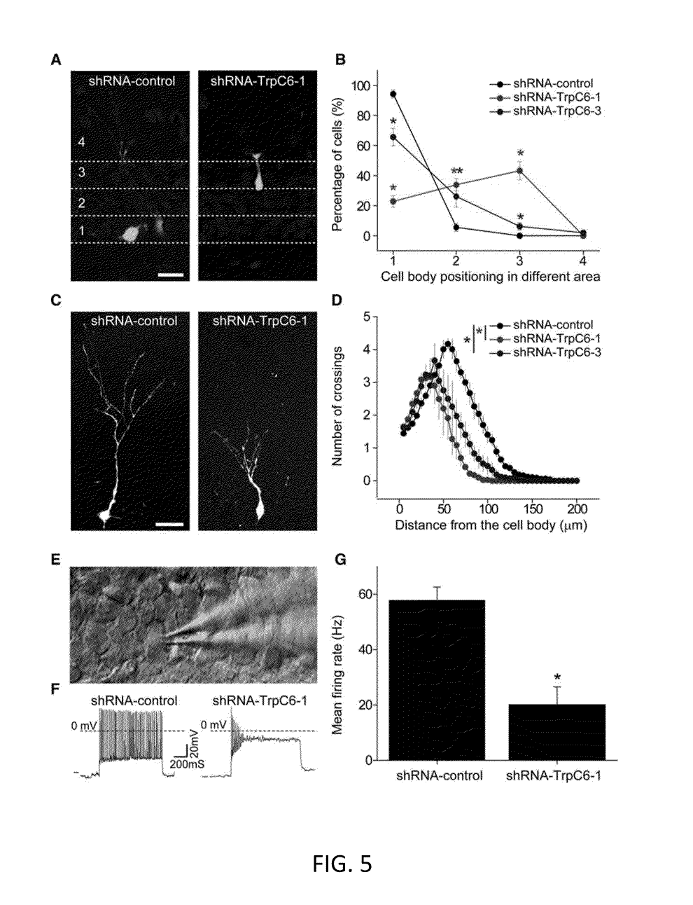

In vitro experiments in rodent primary neurons showed that spine density and excitatory synapses depends on the Trpc6 levels (Leuner et al., 2012; Zhou et al., 2008). Similarly, as we shown above, we also found that downregulation of TRPC6 caused similar neuron alteration in human neurons. We attempted to validate the cell autonomous effect of TRPC6 loss of function in vivo, by taking advantage of adult neurogenesis in the hippocampus (Ming and Song, 2011). Using retroviruses to target newborn neurons, we delivered specific shRNAs against mouse Trpc6. Downregulation of Trpc6 led to migration defects and reduced neuronal dendritic arborization (FIGS. 5A-D and 9A-F). Moreover, whole-cell patch clamping to record action potentials revealed a significant reduction in the firing rate of neurons expressing shRNAs against Trpc6 compared to controls (FIG. 5E-G). TRPC6 knockout (KO) mice (Dietrich et al., 2005) displayed reduced exploratory activity in a square open field and elevated star maze when compared to control siblings (Beis et al.). Limited environmental exploration is commonly associated to ASD patients (Pierce and Courchesne, 2001). Thus, we decided to investigate if the TRPC6 KO mouse displays other ASD-like behavior. We assessed social interaction and repetitive behaviors of these animals, but no significant difference between wild type controls (WT) and heterozygotes (HET) or WT and KO mice was found (FIG. 9G).

### Mutation Screening of TRPC6

Using the initial observation of TRPC6 disruption by a chromosomal breakpoint, we established a narrow hypothesis focusing on TRPC6 to conduct a single gene case/control association study. We screened targeted high-throughput sequencing data from all coding exons and splice sites of TRPC6 in 1041 ASD cases from the Simons Simplex Collection (SSC) (Fischbach and Lord, 2010) and 942 ancestrally-matched controls from the NINDS Neurologically Normal Caucasian Control Panel (http://ccr.coriell.org/Sections/Collections/NINDS/). We focused on novel splice site, missense, and nonsense mutations seen only once across all of our cohorts and not present in dbSNP137 and 6503 exomes available from the Exome Variant Server (EVS, v.0.0.15). We reasoned that these variants were most likely to be deleterious and subject to purifying selection. Moreover, the study of variants observed and counted only once, in combination with case-control matching for ancestry, was thought to represent the most rigorous approach to protecting against population stratification (Mathieson and McVean, 2012). Table S4 lists all such variants in TRPC6. We found a statistically significant increase in novel nonsynonymous singleton mutations in cases compared to controls (10/1041 cases versus 1/942 controls; p=0.013, OR=9.127, 95% CI=1.211-191.027, Fisher exact test, two-tailed). In an effort to confirm the low mutation rate observed in this control sample, we turned to whole exome-sequencing data from an in-house database and found an additional 1930 northern European (NE) controls who clustered tightly with the HapMap CEU cohort. We evaluated the coding exons and splice sites of TRPC6 and, to maximize sensitivity, did not set a minimum read threshold to identify all novel nonsynonymous singleton variants, which are listed in Table S4.

TABLE S4
Novel nonsynonymous singleton mutations in TRPC6.

| | | | | | | | | | |
|---|---|---|---|---|---|---|---|---|---|
| Exon | Variant^{a} | Coordinate (hg19) | Ref > Var | ID | Gender | Father | Mother | Proband | Sib(s) |

TRPC6 variants in cases: SSC probands (n = 1041)

| | | | | | | | | | |
|---|---|---|---|---|---|---|---|---|---|
| 1 | M1K | chr11: 101454233 | A > T | 11561.p1 | M | − | + | + | + (M) |
| 1 | Q3X | chr11: 101454228 | G > A | 12297.p1 | M | + | − | + | + (M) |
| 1 | P47A | chr11: 101454096 | G > C | 13513.p1 | M | + | − | + | + (F) |
| 2 | Y207S | chr11: 101375080 | T > G | 13089.p1 | M | − | + | + | + (F) |
| 3 | L353F | chr11: 101362358 | G > A | 11627.p1 | M | + | − | + | − (F) |
| 5 | P439R | chr11: 101353874 | G > C | 11892.p1 | M | + | − | + | + s1 (F), + s2 (M) |
| 5 | E466K | chr11: 101353794 | C > T | 12646.p1 | M | + | − | + | − s1 (M), − s2 (M) |
| 6 | A560V | chr11: 101347097 | G > A | 11450.p1 | M | − | + | + | + (F) |
| 9 | F795L | chr11: 101341938 | G > C | 11425.p1 | M | − | + | + | − (F) |
| 10 | K808N | chr11: 101340218 | C > A | 11266.p1 | F | − | + | + | − s1 (F), + s2 (F) |

TRPC6 variants in controls: NINDS neurologically normal (n = 942)

| | | | | | | |
|---|---|---|---|---|---|---|
| 3 | M323V | chr11: 101362448 | T > C | ND09598 | F | n/a |

TRPC6 variants in controls: unscreened NE (n = 1930)

| | | | | | | |
|---|---|---|---|---|---|---|
| 7 | I594M | chr11: 101344467 | T > C | S16A11 | F | n/a |
| 8 | A725D | chr11: 101342899 | G > T | S2G9 | M | n/a |
| 9 | A747S | chr11: 101342084 | C > A | S6F6 | M | n/a |

^{a}All variants are heterozygous.

An omnibus analysis revealed an even more significant over-representation of such variants in cases (10/1041 cases versus 4/2872 controls; p=0.001, OR=6.954, 95% CI=2.008-26.321, Fisher exact test, two-tailed). Two of the case variants are especially noteworthy: M1K, which disrupts the start codon, and Q3X, which is a very early premature stop codon, since our results demonstrate that TRPC6 disruption leads to haploinsufficiency of the corresponding protein. No TRPC6 mutations affecting the start codon or nonsense mutations were found in a total of 7445 controls: 942 NINDS neurologically normal Europeans and 6503 exomes from the EVS (4300 European-American, 2203 African-American). Segregation analysis of the case variants revealed that each is inherited from an apparently unaffected parent, suggesting that these variants are incompletely penetrant, as has been previously observed for a wide range of ASD mutations, such as Shank3 (Durand et al., 2007) and CNTNAP2 (Bakkaloglu et al., 2008). Thus, although these genetic variations cannot be considered as causal mutations, they might nevertheless represent risk factors for ASD. Finally, to date, no TRPC6 CNVs have been described in ASD (http://projects.tcag.ca/autism_{—}500k).

### Discussion

A rapidly increasing number of ASD risk regions are being identified, and there is now considerable effort focused on moving from gene discovery to an understanding of the biological substrates influenced by these various mutations (Iossifov et al., 2012; Neale et al., 2012; O'Roak et al., 2012; Sanders et al., 2011; Sanders et al., 2012; Talkowski et al., 2012). The development of relevant human-derived cellular models to study ASDs represents a complementary strategy to link genetic alterations to molecular mechanisms, complex behavioral and cognitive phenotypes (Chailangkarn et al., 2012).

Here, we identified the disruption of the TRPC6 gene by a balanced de novo translocation in an ASD patient. TRPC6 is involved in regulation of axonal guidance, dendritic spine growth and excitatory synapse formation (Li et al., 2005; Tai et al., 2008; Zhou et al., 2008), processes that have been consistently implicated in ASD etiology (Chapleau et al., 2009; Cruz-Martin et al., 2010; Sbacchi et al., 2010; Voineagu et al., 2011). In agreement, the transcriptional analysis conducted in the DPSC of the patient suggested that haploinsufficiency of TRPC6 leads to dysregulation of genes involved in neuronal adhesion, neurite growth and axonal guidance. This abnormal dysregulation is possibly triggered by lower levels of CREB phosphorylation, the transcription factor activated by TRPC6 signaling (Tai et al., 2008). CREB controls a complex regulatory network involved in memory formation, neuronal development and plasticity in the mammalian brain, processes compromised in ASD (Balschun et al., 2003; Dworkin and Mantamadiotis, 2010; Lonze et al., 2002).

Reprogramming our patient's DPSCs to a pluripotent state allowed us to explore the functional consequences of TRPC6 disruption in human neuronal cells. Ca²⁺ influx was aberrant in the patient's NPCs, suggesting that Ca²⁺ signaling-dependent mechanisms were compromised in these cells. Ca²⁺ signaling pathways have already been implicated in ASD etiology since mutations in different voltage-gated Ca⁺² channels and Ca⁺²-regulated signaling molecules have been identified in ASD patients (Hemara-Wahanui et al., 2005; Krey and Dolmetsch, 2007; Splawski et al., 2004; Splawski et al., 2006). Analysis of neurons derived from the patient's iPSCs showed a reduction of VGLUT1 puncta density, in agreement with previous work demonstrating that TRPC6 expression levels can modulate formation of glutamatergic synapses in rat neurons (Zhou et al., 2008). Alterations in glutamatergic neurotransmission have been identified in patients with syndromic forms of ASD: dysregulation of the metabotropic glutamate receptor 1/5 (mGluR1/5) pathway has been well documented in Fragile-X syndrome, and neurons derived from RTT patients' iPSCs also present a reduction in the number of VGLUT1 puncta (Bear et al., 2004; Hagerman et al., 2010; Marchetto et al., 2010). Additionally, a reduction in glutamatergic transmission was observed in Shank3 heterozygous mice, an ASD mouse model (Bozdagi et al., 2010).

Haploinsufficiency of TRPC6 causes other functional and morphological alterations in human neurons that mainly reflect the commitment to axonal and dendritic growth, such as shortening of neurites, decrease in arborization and reduction in dendritic spine density. Due to the high degree of locus heterogeneity, it is challenge to find more patients carrying similar rare variants in the ASD population. Thus, we used complementary functional assays, such as loss-of-function experiments and mouse models, to validate the observation that TRPC6 is important for neuronal homeostasis. Lower spine density was also detected in our previous work in neurons derived from RTT patients (Marchetto et al., 2010). These results suggest that reduction or deregulation of spine density is a common neuronal phenotype in disorders with autistic features and impairment of common pathways must be occurring in these different diseases. Indeed, our data suggest that MeCP2 acts upstream of TRPC6, regulating its expression, as previously reported in the mouse brain (Chahrour et al., 2008; Li et al., 2012). The fact that altered neuronal phenotype is shared among the TRPC6-mutant patient and RTT patients supports the idea that these disorders are caused by different mutational mechanisms affecting common pathways. Additional studies using large samples of idiopathic ASD patients will help to address this hypothesis. Our findings also open a new perspective to test novel drugs in ASD, such as hyperforin, a drug that specifically activates TRPC6 (Leuner et al., 2007; Muller, 2003), or IGF-1. Therefore, patients with alterations in this pathway might benefit from these drugs.

The TRPC6 KO mice present a reduced exploratory interest, a typical ASD-like behavior, but no impaired social interaction or repetitive movements. Lack of some ASD-like behaviors in mouse models is common and can be attributed to the inherent differences between human and mouse genetic backgrounds and neural circuits (Oddi et al., 2013; Silverman et al., 2010; Wohr et al., 2012; Xu et al., 2012). Alternatively, other genetic alterations may be required to develop the full-blown autistic phenotype in this mouse model. The multiple-hit hypothesis is supported by our sequencing findings, revealing TRPC6 loss-of-function mutations in two other ASD patients with incomplete penetrance of the phenotype. A growing number of evidences favoring the multiple-hit model in a significant proportion of ASD patients have been observed in several other circumstances and seems to be the case of the ASD patient described here (Bakkaloglu et al., 2008; Leblond et al., 2012; Marshall et al., 2008; Poot et al., 2010; Poot et al., 2011).

Nonetheless, when viewed in light of our functional data, showing that this gene has a crucial role in synaptogenesis and is involved in a series of mechanisms previously associated with ASD, the mutation screening data suggest that rare TRPC6 variants may contribute to the pathophysiology of the disease, but place limits on the magnitude of this contribution. Thus, the work places TRPC6 as one more rare ASD-correlated gene that likely acts in combination with other genetic variants to contribute to autistic phenotypes. Our work demonstrates that patients' iPSC-derived neurons can be used to correlate novel variants in ASD patients to the etiology of these highly complex disorders.

### REFERENCES FOR EXAMPLE 2

Bakkaloglu, B., O'Roak, B. J., Louvi, A., Gupta, A. R., Abelson, J. F., Morgan, T. M., Chawarska, K., Klin, A., Ercan-Sencicek, A. G., Stillman, A. A., et al. (2008). Molecular cytogenetic analysis and resequencing of contactin associated protein-like 2 in autism spectrum disorders. Am J Hum Genet 82, 165-173.
Balschun, D., Wolfer, D. P., Gass, P., Mantamadiotis, T., Welzl, H., Schutz, G., Frey, J. U., and Lipp, H. P. (2003). Does cAMP response element-binding protein have a pivotal role in hippocampal synaptic plasticity and hippocampus-dependent memory? J Neurosci 23, 6304-6314.
Bear, M. F., Huber, K. M., and Warren, S. T. (2004). The mGluR theory of fragile X mental retardation. Trends Neurosci 27, 370-377.
Beis, D., Schwarting, R. K., and Dietrich, A. Evidence for a supportive role of classical transient receptor potential 6 (TRPC6) in the exploration behavior of mice. Physiol Behav 102, 245-250.
Beltrao-Braga, P. I., Pignatari, G. C., Maiorka, P. C., Oliveira, N. A., Lizier, N. F., Wenceslau, C. V., Miglino, M. A., Muotri, A. R., and Kerkis, I. (2011). Feeder-free derivation of induced pluripotent stem cells from human immature dental pulp stem cells. Cell Transplant.
Bozdagi, O., Sakurai, T., Papapetrou, D., Wang, X., Dickstein, D. L., Takahashi, N., Kajiwara, Y., Yang, M., Katz, A. M., Scattoni, M. L., et al. (2010). Haploinsufficiency of the autism-associated Shank3 gene leads to deficits in synaptic function, social interaction, and social communication. Mol Autism 1, 15.
Campos, M., Jr., Pestana, C. P., dos Santos, A. V., Ponchel, F., Churchman, S., Abdalla-Carvalho, C. B., dos Santos, J. M., dos Santos, F. L., Gikovate, C. G., Santos-Reboucas, C. B., et al. (2011). A MECP2 missense mutation within the MBD domain in a Brazilian male with autistic disorder. Brain Dev 33, 807-809.
Carney, R. M., Wolpert, C. M., Ravan, S. A., Shahbazian, M., Ashley-Koch, A., Cuccaro, M. L., Vance, J. M., and Pericak-Vance, M. A. (2003). Identification of MeCP2 mutations in a series of females with autistic disorder. Pediatr Neurol 28, 205-211.
Chahrour, M., Jung, S. Y., Shaw, C., Zhou, X., Wong, S. T., Qin, J., and Zoghbi, H. Y. (2008). MeCP2, a key contributor to neurological disease, activates and represses transcription. Science 320, 1224-1229.
Chailangkarn, T., Acab, A., and Muotri, A. R. (2012). Modeling neurodevelopmental disorders using human neurons. Curr Opin Neurobiol.
Chapleau, C. A., Larimore, J. L., Theibert, A., and Pozzo-Miller, L. (2009). Modulation of dendritic spine development and plasticity by BDNF and vesicular trafficking: fundamental roles in neurodevelopmental disorders associated with mental retardation and autism. J Neurodev Disord 1, 185-196.
Colella, S., Yau, C., Taylor, J. M., Mirza, G., Butler, H., Clouston, P., Bassett, A. S., Seller, A., Holmes, C. C., and Ragoussis, J. (2007). QuantiSNP: an Objective Bayes Hidden-Markov Model to detect and accurately map copy number variation using SNP genotyping data. Nucleic Acids Res 35, 2013-2025.
Cruz-Martin, A., Crespo, M., and Portera-Cailliau, C. (2010). Delayed stabilization of dendritic spines in fragile X mice. J Neurosci 30, 7793-7803.
Cukier, H. N., Lee, J. M., Ma, D., Young, J. I., Mayo, V., Butler, B. L., Ramsook, S. S., Rantus, J. A., Abrams, A. J., Whitehead, P. L., et al. (2012). The Expanding Role of MBD Genes in Autism: Identification of a MECP2 Duplication and Novel Alterations in MBD5, MBD6, and SETDB1. Autism Res.
Cukier, H. N., Rabionet, R., Konidari, I., Rayner-Evans, M. Y., Baltos, M. L., Wright, H. H., Abramson, R. K., Martin, E. R., Cuccaro, M. L., Pericak-Vance, M. A., et al. (2010). Novel variants identified in methyl-CpG-binding domain genes in autistic individuals. Neurogenetics 11, 291-303.
d'Aquino, R., De Rosa, A., Laino, G., Caruso, F., Guida, L., Rullo, R., Checchi, V., Laino, L., Tirino, V., and Papaccio, G. (2009). Human dental pulp stem cells: from biology to clinical applications. J Exp Zool B Mol Dev Evol 312B, 408-415.
Dietrich, A., Mederos, Y. S. M., Gollasch, M., Gross, V., Storch, U., Dubrovska, G., Obst, M., Yildirim, E., Salanova, B., Kalwa, H., et al. (2005). Increased vascular smooth muscle contractility in TRPC6−/− mice. Mol Cell Biol 25, 6980-6989.
Dotti, M. T., Orrico, A., De Stefano, N., Battisti, C., Sicurelli, F., Severi, S., Lam, C. W., Galli, L., Sorrentino, V., and Federico, A. (2002). A Rett syndrome MECP2 mutation that causes mental retardation in men. Neurology 58, 226-230.
Duan, X., Chang, J. H., Ge, S., Faulkner, R. L., Kim, J. Y., Kitabatake, Y., Liu, X. B., Yang, C. H., Jordan, J. D., Ma, D. K., et al. (2007). Disrupted-In-Schizophrenia 1 regulates integration of newly generated neurons in the adult brain. Cell 130, 1146-1158.
Durand, C. M., Betancur, C., Boeckers, T. M., Bockmann, J., Chaste, P., Fauchereau, F., Nygren, G., Rastam, M., Gillberg, I. C., Anckarsater, H., et al. (2007). Mutations in the gene encoding the synaptic scaffolding protein SHANK3 are associated with autism spectrum disorders. Nat Genet 39, 25-27.
Dworkin, S., and Mantamadiotis, T. (2010). Targeting CREB signalling in neurogenesis. Expert Opin Ther Targets 14, 869-879.
Fiorio Pla, A., Maric, D., Brazer, S. C., Giacobini, P., Liu, X., Chang, Y. H., Ambudkar, I. S., and Barker, J. L. (2005). Canonical transient receptor potential 1 plays a role in basic fibroblast growth factor (bFGF)/FGF receptor-1-induced Ca2+ entry and embryonic rat neural stem cell proliferation. J Neurosci 25, 2687-2701.
Fischbach, G. D., and Lord, C. (2010). The Simons Simplex Collection: a resource for identification of autism genetic risk factors. Neuron 68, 192-195.
Ge, S., Goh, E. L., Sailor, K. A., Kitabatake, Y., Ming, G. L., and Song, H. (2006). GABA regulates synaptic integration of newly generated neurons in the adult brain. Nature 439, 589-593.
Gronthos, S., Brahim, J., Li, W., Fisher, L. W., Cheman, N., Boyde, A., DenBesten, P., Robey, P. G., and Shi, S. (2002). Stem cell properties of human dental pulp stem cells. J Dent Res 81, 531-535.
Hagerman, R., Hoem, G., and Hagerman, P. (2010). Fragile X and autism: Intertwined at the molecular level leading to targeted treatments. Mol Autism 1, 12.
Han, S., Tai, C., Westenbroek, R. E., Yu, F. H., Cheah, C. S., Potter, G. B., Rubenstein, J. L., Scheuer, T., de la Iglesia, H. O., and Catterall, W. A. (2012). Autistic-like behaviour in Scn1a+/− mice and rescue by enhanced GABA-mediated neurotransmission. Nature 489, 385-390.
Hemara-Wahanui, A., Berjukow, S., Hope, C. I., Dearden, P. K., Wu, S. B., Wilson-Wheeler, J., Sharp, D. M., Lundon-Treweek, P., Clover, G. M., Hoda, J. C., et al. (2005). A CACNA1F mutation identified in an X-linked retinal disorder shifts the voltage dependence of Cav1.4 channel activation. Proc Natl Acad Sci USA 102, 7553-7558.
Iossifov, I., Ronemus, M., Levy, D., Wang, Z., Hakker, I., Rosenbaum, J., Yamrom, B., Lee, Y. H., Narzisi, G., Leotta, A., et al. (2012). De novo gene disruptions in children on the autistic spectrum. Neuron 74, 285-299.
Irizarry, R. A., Hobbs, B., Collin, F., Beazer-Barclay, Y. D., Antonellis, K. J., Scherf, U., and Speed, T. P. (2003). Exploration, normalization, and summaries of high density oligonucleotide array probe level data. Biostatistics 4, 249-264.
Jia, Y., Zhou, J., Tai, Y., and Wang, Y. (2007). TRPC channels promote cerebellar granule neuron survival. Nat Neurosci 10, 559-567.
Kang, E., Burdick, K. E., Kim, J. Y., Duan, X., Guo, J. U., Sailor, K. A., Jung, D. E., Ganesan, S., Choi, S., Pradhan, D., et al. (2011). Interaction between FEZ1 and DISC1 in regulation of neuronal development and risk for schizophrenia. Neuron 72, 559-571.
Kim, J. Y., Duan, X., Liu, C. Y., Jong, M. H., Guo, J. U., Pow-anpongkul, N., Kang, E., Song, H., and Ming, G. L. (2009). DISC1 regulates new neuron development in the adult brain via modulation of AKT-mTOR signaling through KIAA1212. Neuron 63, 761-773.
Krey, J. F., and Dolmetsch, R. E. (2007). Molecular mechanisms of autism: a possible role for Ca2+ signaling. Curr Opin Neurobiol 17, 112-119.
Kuwano, Y., Kamio, Y., Kawai, T., Katsuura, S., Inada, N., Takaki, A., and Rokutan, K. (2011). Autism-associated gene expression in peripheral leucocytes commonly observed between subjects with autism and healthy women having autistic children. PLoS One 6, e24723.
Lam, C. W., Yeung, W. L., Ko, C. H., Poon, P. M., Tong, S. F., Chan, K. Y., Lo, I. F., Chan, L. Y., Hui, J., Wong, V., et al. (2000). Spectrum of mutations in the MECP2 gene in patients with infantile autism and Rett syndrome. J Med Genet 37, E41.
Leblond, C. S., Heinrich, J., Delorme, R., Proepper, C., Betancur, C., Huguet, G., Konyukh, M., Chaste, P., Ey, E., Rastam, M., et al. (2012). Genetic and functional analyses of SHANK2 mutations suggest a multiple hit model of autism spectrum disorders. PLoS genetics 8, e1002521.
Leuner, K., Heiser, J. H., Derksen, S., Mladenov, M. I., Fehske, C. J., Schubert, R., Gollasch, M., Schneider, G., Harteneck, C., Chatterjee, S. S., et al. (2010). Simple 2,4-diacylphloroglucinols as classic transient receptor potential-6 activators—identification of a novel pharmacophore. Mol Pharmacol 77, 368-377.
Leuner, K., Kazanski, V., Muller, M., Essin, K., Henke, B., Gollasch, M., Harteneck, C., and Muller, W. E. (2007). Hyperforin—a key constituent of St. John's wort specifically activates TRPC6 channels. Faseb J 21, 4101-4111.
Leuner, K., Li, W., Amaral, M. D., Rudolph, S., Calfa, G., Schuwald, A. M., Harteneck, C., Inoue, T., and Pozzo-Miller, L. (2012). Hyperforin modulates dendritic spine morphology in hippocampal pyramidal neurons by activating Ca(2+)-permeable TRPC6 channels. Hippocampus.
Li, W., Calfa, G., Larimore, J., and Pozzo-Miller, L. (2012). Activity-dependent BDNF release and TRPC signaling is impaired in hippocampal neurons of Mecp2 mutant mice. Proc Natl Acad Sci USA.
Li, Y., Jia, Y. C., Cui, K., Li, N., Zheng, Z. Y., Wang, Y. Z., and Yuan, X. B. (2005). Essential role of TRPC channels in the guidance of nerve growth cones by brain-derived neurotrophic factor. Nature 434, 894-898.
Lichter, P., Tang, C. J., Call, K., Hermanson, G., Evans, G. A., Housman, D., and Ward, D. C. (1990). High-resolution mapping of human chromosome 11 by in situ hybridization with cosmid clones. Science 247, 64-69.
Livak, K. J., and Schmittgen, T. D. (2001). Analysis of relative gene expression data using real-time quantitative PCR and the 2(-Delta Delta C(T)) Method. Methods 25, 402-408.
Lonze, B. E., Riccio, A., Cohen, S., and Ginty, D. D. (2002). Apoptosis, axonal growth defects, and degeneration of peripheral neurons in mice lacking CREB. Neuron 34, 371-385.
Marchetto, M. C., Carromeu, C., Acab, A., Yu, D., Yeo, G. W., Mu, Y., Chen, G., Gage, F. H., and Muotri, A. R. (2010). A model for neural development and treatment of Rett syndrome using human induced pluripotent stem cells. Cell 143, 527-539.
Marshall, C. R., Noor, A., Vincent, J. B., Lionel, A. C., Feuk, L., Skaug, J., Shago, M., Moessner, R., Pinto, D., Ren, Y., et al. (2008). Structural variation of chromosomes in autism spectrum disorder. Am J Hum Genet 82, 477-488.
Mathieson, I., and McVean, G. (2012). Differential confounding of rare and common variants in spatially structured populations. Nat Genet 44, 243-246.
McCall, C. M., Miliani de Marval, P. L., Chastain, P. D., 2nd, Jackson, S. C., He, Y. J., Kotake, Y., Cook, J. G., and Xiong, Y. (2008). Human immunodeficiency virus type 1 Vpr-binding protein VprBP, a WD40 protein associated with the DDB1-CUL4 E3 ubiquitin ligase, is essential for DNA replication and embryonic development. Mol Cell Biol 28, 5621-5633.
Ming, G. L., and Song, H. (2011). Adult neurogenesis in the mammalian brain: significant answers and significant questions. Neuron 70, 687-702.
Miura, M., Gronthos, S., Zhao, M., Lu, B., Fisher, L. W., Robey, P. G., and Shi, S. (2003). SHED: stem cells from human exfoliated deciduous teeth. Proc Natl Acad Sci USA 100, 5807-5812.
Muller, M., Essin, K., Hill, K., Beschmann, H., Rubant, S., Schempp, C. M., Gollasch, M., Boehncke, W. H., Harteneck, C., Muller, W. E., et al. (2008). Specific TRPC6 channel activation, a novel approach to stimulate keratinocyte differentiation. J Biol Chem 283, 33942-33954.
Muller, W. E. (2003). Current St John's wort research from mode of action to clinical efficacy. Pharmacol Res 47, 101-109.
Muotri, A. R., Marchetto, M. C., Coufal, N. G., Oefner, R., Yeo, G., Nakashima, K., and Gage, F. H. (2010). L1 retrotransposition in neurons is modulated by MeCP2. Nature 468, 443-446.
Nagarajan, R. P., Hogart, A. R., Gwye, Y., Martin, M. R., and LaSalle, J. M. (2006). Reduced MeCP2 expression is frequent in autism frontal cortex and correlates with aberrant MECP2 promoter methylation. Epigenetics 1, e1-11.
Neale, B. M., Kou, Y., Liu, L., Ma'ayan, A., Samocha, K. E., Sabo, A., Lin, C. F., Stevens, C., Wang, L. S., Makarov, V., et al. (2012). Patterns and rates of exonic de novo mutations in autism spectrum disorders. Nature 485, 242-245.
O'Roak, B. J., Vives, L., Girirajan, S., Karakoc, E., Krumm, N., Coe, B. P., Levy, R., Ko, A., Lee, C., Smith, J. D., et al. (2012). Sporadic autism exomes reveal a highly interconnected protein network of de novo mutations. Nature 485, 246-250.
Oddi, D., Crusio, W. E., D'Amato, F. R., and Pietropaolo, S. (2013). Monogenic mouse models of social dysfunction: Implications for autism. Behav Brain Res.
Pierce, K., and Courchesne, E. (2001). Evidence for a cerebellar role in reduced exploration and stereotyped behavior in autism. Biol Psychiatry 49, 655-664.
Piton, A., Gauthier, J., Hamdan, F. F., Lafreniere, R. G., Yang, Y., Henrion, E., Laurent, S., Noreau, A., Thibodeau, P., Karemera, L., et al. (2011). Systematic resequencing of X-chromosome synaptic genes in autism spectrum disorder and schizophrenia. Mol Psychiatry 16, 867-880.
Poot, M., Beyer, V., Schwaab, I., Damatova, N., Van't Slot, R., Prothero, J., Holder, S. E., and Haaf, T. (2010). Disruption of CNTNAP2 and additional structural genome changes in a boy with speech delay and autism spectrum disorder. Neurogenetics 11, 81-89.
Poot, M., van der Smagt, J. J., Brilstra, E. H., and Bourgeron, T. (2011). Disentangling the myriad genomics of complex disorders, specifically focusing on autism, epilepsy, and schizophrenia. Cytogenet Genome Res 135, 228-240.
Samaco, R. C., Nagarajan, R. P., Braunschweig, D., and LaSalle, J. M. (2004). Multiple pathways regulate MeCP2 expression in normal brain development and exhibit defects in autism-spectrum disorders. Hum Mol Genet 13, 629-639.
Sanders, S. J., Ercan-Sencicek, A. G., Hus, V., Luo, R., Murtha, M. T., Moreno-De-Luca, D., Chu, S. H., Moreau, M. P., Gupta, A. R., Thomson, S. A., et al. (2011). Multiple recurrent de novo CNVs, including duplications of the 7q11.23 Williams syndrome region, are strongly associated with autism. Neuron 70, 863-885.
Sanders, S. J., Murtha, M. T., Gupta, A. R., Murdoch, J. D., Raubeson, M. J., Willsey, A. J., Ercan-Sencicek, A. G., DiLullo, N. M., Parikshak, N, N., Stein, J. L., et al. (2012). De novo mutations revealed by whole-exome sequencing are strongly associated with autism. Nature 485, 237-241.
Sbacchi, S., Acquadro, F., Cabo, I., Cali, F., and Romano, V. (2010). Functional annotation of genes overlapping copy number variants in autistic patients: focus on axon pathfinding. Curr Genomics 11, 136-145.
Silverman, J. L., Yang, M., Lord, C., and Crawley, J. N. (2010). Behavioural phenotyping assays for mouse models of autism. Nat Rev Neurosci 11, 490-502.
Splawski, I., Timothy, K. W., Sharpe, L. M., Decher, N., Kumar, P., Bloise, R., Napolitano, C., Schwartz, P. J., Joseph, R. M., Condouris, K., et al. (2004). Ca(V)1.2 calcium channel dysfunction causes a multisystem disorder including arrhythmia and autism. Cell 119, 19-31.
Splawski, I., Yoo, D. S., Stotz, S. C., Chemy, A., Clapham, D. E., and Keating, M. T. (2006). CACNA1H mutations in autism spectrum disorders. J Biol Chem 281, 22085-22091.
State, M. W., and Levitt, P. (2011). The conundrums of understanding genetic risks for autism spectrum disorders. Nat Neurosci 14, 1499-1506.
Tai, Y., Feng, S., Du, W., and Wang, Y. (2009). Functional roles of TRPC channels in the developing brain. Pflugers Arch 458, 283-289.
Tai, Y., Feng, S., Ge, R., Du, W., Zhang, X., He, Z., and Wang, Y. (2008). TRPC6 channels promote dendritic growth via the CaMKIV-CREB pathway. J Cell Sci 121, 2301-2307.
Takahashi, K., Tanabe, K., Ohnuki, M., Narita, M., Ichisaka, T., Tomoda, K., and Yamanaka, S. (2007). Induction of pluripotent stem cells from adult human fibroblasts by defined factors. Cell 131, 861-872.
Talkowski, M. E., Rosenfeld, J. A., Blumenthal, I., Pillalamarri, V., Chiang, C., Heilbut, A., Ernst, C., Hanscom, C., Rossin, E., Lindgren, A. M., et al. (2012). Sequencing chromosomal abnormalities reveals neurodevelopmental loci that confer risk across diagnostic boundaries. Cell 149, 525-537.
Tropea, D., Giacometti, E., Wilson, N. R., Beard, C., McCurry, C., Fu, D. D., Flannery, R., Jaenisch, R., and Sur, M. (2009). Partial reversal of Rett Syndrome-like symptoms in MeCP2 mutant mice. Proc Natl Acad Sci USA 106, 2029-2034.
Tusher, V. G., Tibshirani, R., and Chu, G. (2001). Significance analysis of microarrays applied to the ionizing radiation response. Proc Natl Acad Sci USA 98, 5116-5121.
Vandesompele, J., De Preter, K., Pattyn, F., Poppe, B., Van Roy, N., De Paepe, A., and Speleman, F. (2002). Accurate normalization of real-time quantitative RT-PCR data by geometric averaging of multiple internal control genes. Genome Biol 3, RESEARCH0034.
Voineagu, I., Wang, X., Johnston, P., Lowe, J. K., Tian, Y., Horvath, S., Mill, J., Cantor, R. M., Blencowe, B. J., and Geschwind, D. H. (2011). Transcriptomic analysis of autistic brain reveals convergent molecular pathology. Nature 474, 380-384.
Wang, K., Li, M., Hadley, D., Liu, R., Glessner, J., Grant, S. F., Hakonarson, H., and Bucan, M. (2007). PennCNV: an integrated hidden Markov model designed for high-resolution copy number variation detection in whole-genome SNP genotyping data. Genome Res 17, 1665-1674.
Wohr, M., Silverman, J. L., Scattoni, M. L., Turner, S. M., Harris, M. J., Saxena, R., and Crawley, J. N. (2012). Developmental delays and reduced pup ultrasonic vocalizations but normal sociability in mice lacking the postsynaptic cell adhesion protein neuroligin2. Behav Brain Res.
Xu, J. Y., Xia, Q. Q., and Xia, J. (2012). A review on the current neuroligin mouse models. Sheng Li Xue Bao 64, 550-562.
Zhang, X., Odom, D. T., Koo, S. H., Conkright, M. D., Canettieri, G., Best, J., Chen, H., Jenner, R., Herbolsheimer, E., Jacobsen, E., et al. (2005). Genome-wide analysis of cAMP-response element binding protein occupancy, phosphorylation, and target gene activation in human tissues. Proc Natl Acad Sci USA 102, 4459-4464.
Zhou, J., Du, W., Zhou, K., Tai, Y., Yao, H., Jia, Y., Ding, Y., and Wang, Y. (2008). Critical role of TRPC6 channels in the formation of excitatory synapses. Nat Neurosci 11, 741-743.

### Supplemental References

Purcell, S., Neale, B., Todd-Brown, K., Thomas, L., Ferreira, M. A., Bender, D., Maller, J., Sklar, P., de Bakker, P. I., Daly, M. J., et al. (2007). PLINK: a tool set for whole-genome association and population-based linkage analyses. Am J Hum Genet 81, 559-575.

### Example 3

### Methods

### IPSC Generation and Characterization.

Male fibroblasts carrying two distinct MeCP2 mutations and two controls (from their respective non-affected fathers) were obtained from explants of dermal biopsies following informed consent under protocols approved by the University of California, San Diego. Retrovirus infection and iPSC generation were performed as previously described³.

Neuronal Induction of iPSCs.

The protocol for neuronal differentiation was based on our previous work with modifications³. The culture media for iPSCs was switched to N2 media with dual SMAD inhibitors (Dorsomorphin and SB) concentrations and, after 48 hours, the iPSCs were placed in suspension as embryoid bodies (EBs) with constant shaking (90 rpm). EBs were kept in N2 media with dual SMAD inhibitors for 72 hours and then plated on Matrigel plates with NG media.

Generation of human iPSC-derived astrocytes.

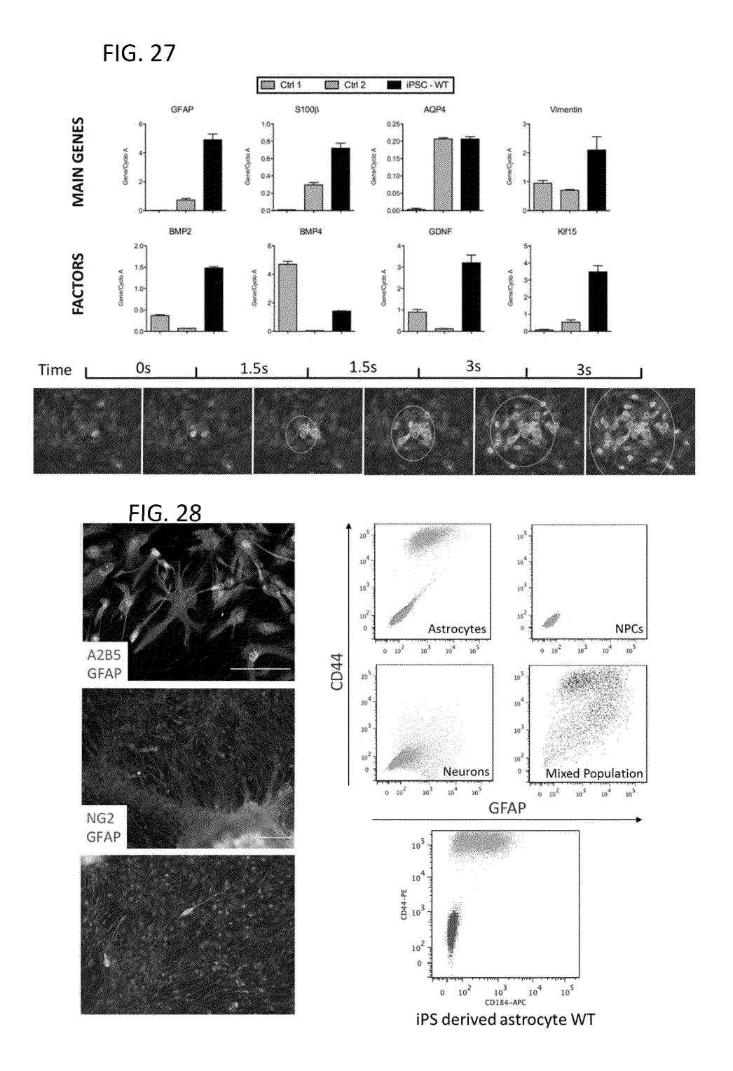

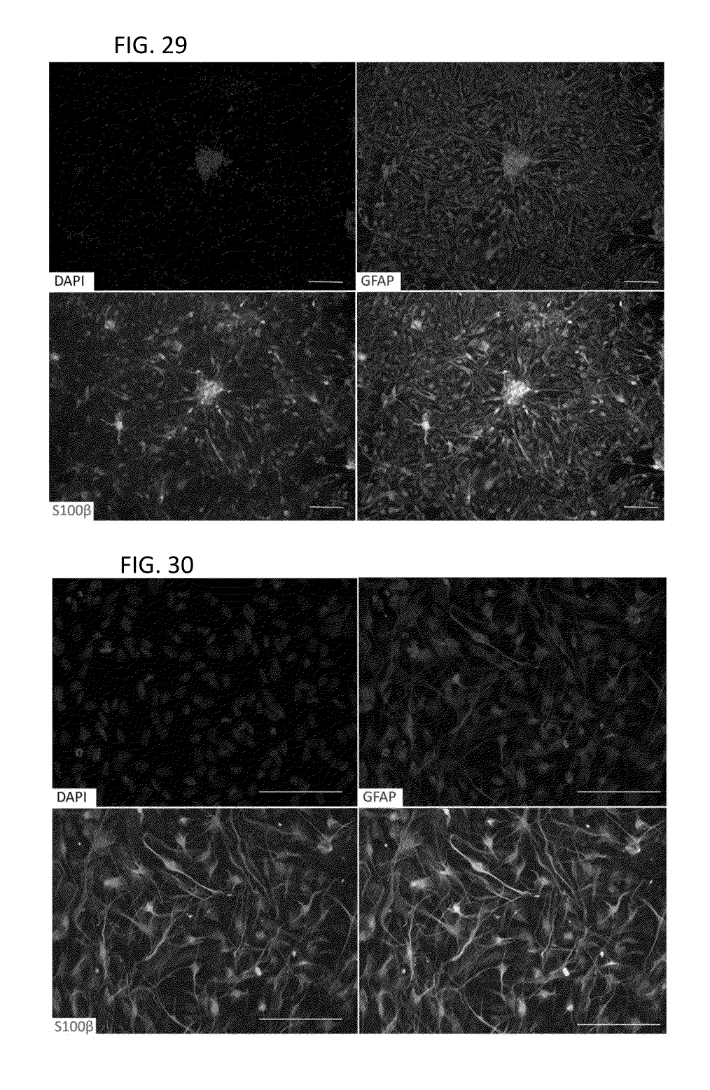

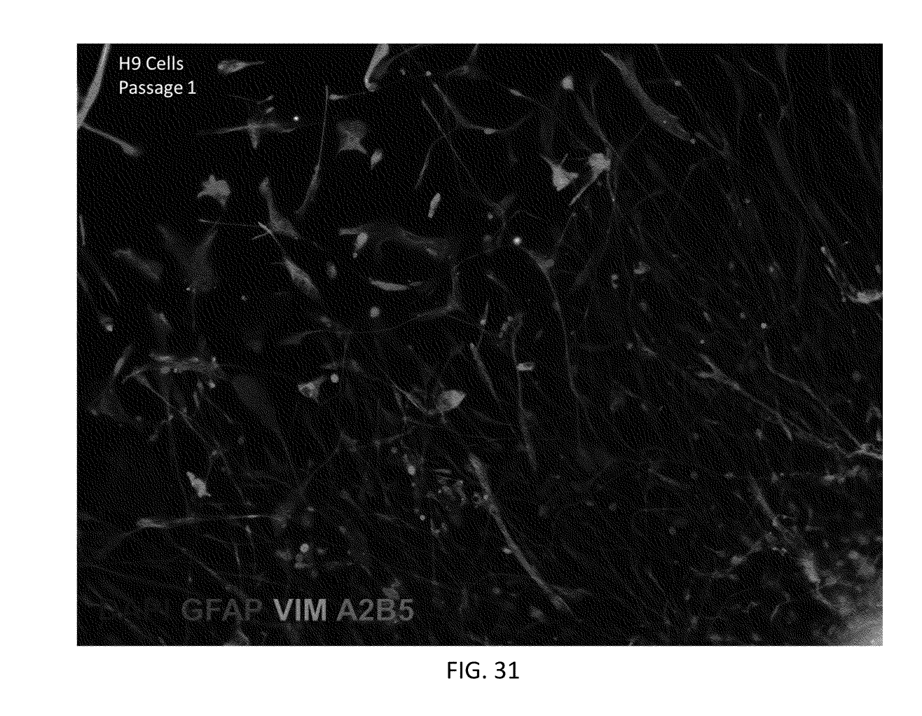

NPCs were derived from iPSCs as described previously³. NPCs from a confluent 100 mm diameter plate were incubated with PBS at 37° C. for 5 minutes and then scraped to form neurospheres using 9 mL of Neuronal media (1:1 N2 and B27) plus FGF, distributed in 3 mL per well in a six-well tissue culture coated plate. Cells were incubated with constant shaking (90 rpm). Media was changed as needed and once the neurospheres were well formed. Rock inhibitor was added to a final concentration of 5 μM for 48 hours concomitant with the removal of FGF from the media. After the removal of Rock inhibitor, neuronal media without FGF was used for one week. Next, astrocyte growth media (Lonza, Allendale, N.J.) was added to the spheres for two weeks with shaking Once the astrocytes started to differentiate from progenitor cells, they began to produce laminin. Astrocytes attached to the bottom of the plate. After two weeks of induction, when cells were found attached to the bottom of the six wells, the astrocytes were plated. Cells attached to the bottom of the six wells were discarded. The supernatant with the remaining spheres was plated in a double-coated plate (polyornithine and laminin), as instructed by the manufacturer, and kept in astrocyte growth media for cell expansion. When the spheres were plated, the astrocytes began to grow out of the sphere and spread on the plate to form a multilayer cell formation. At the first splitting of the astrocytes, the spheres were removed to generate a more pure population of cells, but the lack of neuronal signaling in the media generates astrocytes (FIGS. 27-31).

### Magnetic Cell Sorting.

Astrocytes were sorted using PE-conjugated anti-CD44 antibodies, and neurons were negatively sorted using PE-conjugated antibodies against CD184 and CD44. Cells were resuspended with Accutase (Cellgro), washed once with PBS, and separated into single cells using a pre-separation filter of 30 μm (MACS). Cells were incubated with the necessary antibodies for 15 minutes in the dark at 4° C. and washed twice with PBS to remove unbound antibodies before sorting.

### RNA Extraction and RT-PCR.

Total cellular RNA was extracted from approximately 5×10⁶ cells, either unsorted or sorted for CD markers. Cells were extracted using Trizol Reagent (Life Technologies, CA) according to the manufacturer's suggestions.

Neural Induction of iPSCs.

The protocol for neuronal differentiation was based in our previous work (Marchetto, et al. 2010) with few modifications. iPSCs have their media replaced to N2 media with dual SMAD inhibitors (Dorsomorphin and SB) and after 48 hours they are placed in suspension as embryoid bodies (EBs) on constant shaking (90 rpm). EBs are kept for 72 hours in N2 media with dual SMAD inhibitors then plated in matrigel plates with NG media.

### Quantitative PCR.

RNA was collected from 4 weeks old neural cultures of RTT and controls using RLT buffer and the extraction performed as described in manufacturer instructions (RNeasy Plus Kit, Qiagen). For cDNA conversion, 100 ng of pure RNA was used as template, following the instructions of the SuperScript II Reverse Transcriptase kit (Life Technologies). The resulting cDNA was subjected to standard quantitative PCR using Fast SYBR green Master Mix (Life Technologies). Each sample was run in triplicate in a 96-well format 7900HT Fast Real-Time PCR system (Applied Biosystems). The primers for the following genes were used: BDNF, NGFR, Fox-3 (NeuN), PSD-95, VGlut1, Synapsin 1, IGF1, EAAT2 and EAAT4. GAPDH was used as control. Primers sequences are listed in the supplementary table S7.

TABLE S7

| Gene | Primer forward | Primer reverse |
|---|---|---|
| BDNF | GGCTTGACATCATT | GCCGAACTTTCTGGTCCTCAT |
| | GGCTGAC | |
| EAAT2 | CCTGACGGTGTTTG | CAAGCGGCCACTAGCCTTAG |
| (SLC1A2) | GTGTCAT | |
| EAAT4 | ACAGTTCAAGACGC | CCAAGGCCCGAGTGACATTTT |
| (SLC1A6) | AGTACAG | |
| Fox-3 | ACTTACGGAGCGGT | GGCTGCGTAGCCTCCATAAA |
| (NeuN) | CGTGTAT | |
| GAPDH | AGGTCGGTGTGAAC | TGTAGACCATGTAGTTGAGGTCA |
| | GGATTTG | |
| IGF1 | GCTCTTCAGTTCGT | CGACTGCTGGAGCCATACC |
| | GTGTGGA | |
| NGFR | CCTACGGCTACTAC | CACACGGTGTTCTGCTTGT |
| | CAGGATG | |
| PSD-95 | TCGGTGACGACCCA | GCACGTCCACTTCATTTACAAAC |
| | TCCAT | |
| Synapin 1 | TGCTCAGCAGTACA | GACACTTGCGATGTCCTGGAA |
| | ACGTACC | |
| VGlut1 | CAGAGTTTTCGGCT | GCGACTCCGTTCTAAGGGTG |
| (SLC17A7) | TTGCTATTG | |

### Quantitative PCR of Astrocyte Related Genes.

RNA was collected from passages three or four of each culture, RTT or WT, previously immunocharacterized with main astrocyte markers: GFAP, S100β, vimentin and AQP4. Total RNA was extracted using the Trizol method (Life Technologies). Reverse transcriptase was performed using 3.0 μg of total RNA in the SuperScript First-Strand Synthesis System. Quantitative real-time PCR was performed using Fast SYBR green Master Mix (Life Technologies) using primers designed for specific genes using Primer3 software (v. 0.4.0) or available at the Harvard Primer Bank online. The PCR cycle conditions were as follows: 2 minutes, 30 seconds at 95° C., 30 seconds at 95° C., 30 seconds at 60° C., and 45 seconds at 72° C. for 50 cycles followed by the melting curve protocol to verify the specificity of amplicon generation. Gene expression was determined by generation of a standard curve using a mix of the cDNA from all samples used, and the analysis is made by relative expression with the housekeeping genes Cyclophilin A and GAPDH.

### Western Blotting.

Standard Western blotting techniques were used using the Odyssey machine (LiCor) for quantification. The following antibodies were used: anti-PSD-95 (mouse, NeuroMab); anti-VGlut1 (rabbit, Synaptic Systems); anti-NGFR (rabbit, Abcam); anti-Map2 (mouse, Sigma); anti-synapsin (rabbit, Millipore); anti-GAPDH (mouse, Sigma). Briefly, protein extracts from 4 week old neurons (RTT and controls) were collected with RIPA buffer (Pierce) supplemented with Proteinase Inhibitors (Roche). After homogenization, 10 μg of extract was loaded in a gradient SDS-PAGE for detection. After transfer to nitrocellulose and blocking, primary antibody was incubated overnight and secondary was incubated for 1 h in the next day. There were 5 washes with PBS-Tween 0.1% after each antibody.

### Karyotyping.

G-banding karyotype was performed at Molecular Diagnostic Services, Inc. (San Diego, Calif.). Cells were provided at subconfluent stage. This procedure was repeated every 10 passages of the iPSC and cell lines with abnormal karyotype were discarded.

### Multi-Electrode Array.

To check connectivity in RTT and control neural cultures, MED64 multi-electrode array (MEA) system from Panasonic was used. From a confluent 100 mm diameter plate of NPCs, cells were incubated with DPBS (minus Calcium and Magnesium) at 37° C. for 5 minutes and scraped out to form neurospheres using 9 mL of Neuronal media (N2:B27/1:1) plus FGF, being distributed in 3 mL per well in a six well plate. Cells were kept in the shaker with 90 rpm constantly inside a 37° C. incubator. Media was changed as needed and once the neurospheres were well formed, Rock inhibitor was added to a final concentration of 5 μM for 48 hours concomitant with the removal of FGF of the media. After the removal of Rock inhibitor, the media was changed back to neuronal media without FGF for a week. The spheres were then seeded on poly-ornithine/Laminin coated MEA and kept in the 37° C. incubator for additional 2 weeks. After this time, astrocytes and neurons spread all over the electrodes from the original neuropheres and a mature neuronal network was observed. Spontaneous field potential was recorded online using the software Mobius (Panasonic). A 0.03 mV threshold for spike detection was used. Extracted spikes were converted to raster plot and histogram with the software NeuroExplorer (Nex Technologies). For synchronization analyzes, synchronized burst were counted over a 5 minute interval and plotted using the Prism5 software (GraphPad). As definition, we count as a synchronized burst if a burst (at least 10 Hz frequency in a 5 sec interval) is present simultaneously in 4 different channels, implying connectivity between the channels.

Generation of Human iPS-Derived Astrocytes.

Initial steps are described in the multi-electrode array assay, and consistent with the generation and neuronal induction of neurospheres. After one week without FGF, the spheres have their media changed to astrocyte growth media (Lonza, Allendale, N.J.) for two weeks still under rotation on the shaker. Once the astrocytes start differentiating from progenitor cells, they begin to produce laminin, and even with the constant moving they can attach to the bottom of the plate. At this point after two weeks of induction when cells are found in the bottom of the six wells attached, the astrocytes are ready to be plated. The cells that attached to the bottom of the six well were discarded. The supernatant with the remaining spheres was plated in a double coated plate (poly ornithine and laminin as instructed by the manufacturer) and kept in astrocyte growth media for the cell expansion. When the spheres are plated, the astrocytes begin to grow from the spheres and spread in the plate to form a stable multilayer cell formation, capable of being stable in the same dish for up to six months. On the first splitting of the astrocytes, the spheres can be removed for more pure population of cells, but the lack of neuronal signaling in the media makes the astrocytes decrease in the capability of propagation allowing them to be split only a couple times after neurosphere removal.

### Magnetic Cell Sorting.

Astrocytes were sorted using CD44 PE conjugated antibodies and neurons were negatively sorted using CD184 and CD44, both also PE conjugated antibodies. Cells were ressuspended with accutase (Cellgro), washed once with PBS and made into single cells using a pre-separation filter of 30 μm (MACS). Cells were incubated with the antibodies to be sorted for 15 minutes in the dark at 4° C. and washed twice with PBS to remove the excess of antibodies

### RNA Extraction and RT-PCR.

Total cellular RNA was extracted from approximately 5×10⁶ cells unsorted or sorted for CD markers. Cells were extracted using Trizol Reagent (Life Technologies, CA).

In order to generate RTT-iPSCs models without potential problems with X chromosome inactivation and to have a homogenous population of MeCP2 mutant cells, we reprogrammed skin fibroblasts from two male patients and their respective non-affected fathers as controls. RTT-derived neurons from these male iPSCs showed similar phenotypes. Next, we investigate if RTT astrocytes derived from iPSCs are different from controls.

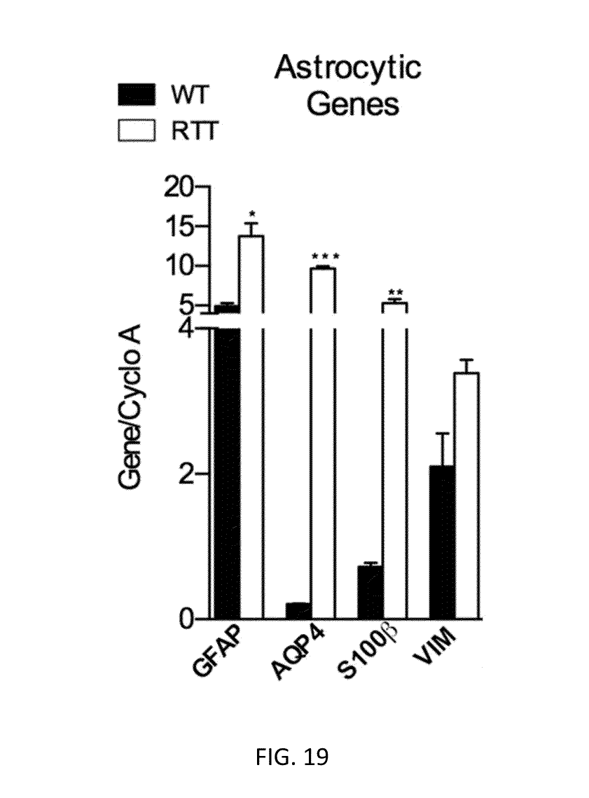

Our protocol for generating astrocytes relies on the natural course of astrocyte maturation. During neurogenesis in the developing brain, the proliferation of GFAP-positive cells is initiated, giving rise to mature astrocytes. Thus, our protocol take advantage of neurospheres that continuously secrete factors that stabilize astrocyte maintenance. FIG. 19 shows the efficiency of our differentiation protocol, as determined by the amount of cells expressing GFAP and vimentin, among other astrocyte markers. Expression of the glial progenitor A2B5 was observed in the perinuclear region of some GFAP-positive cells at early stages. SOX2 expression was observed in the outer layers of the neurospheres and also in the majority of Aldh1L1-positive cells arising from the sphere. The majority of NG2-positive cells were within the neurospheres alongside MAP2-positive neurons. After the first passage, cells surrounding the neurospheres are dissociated enzymatically and plated. The neurospheres are removed and the result is a confluent and homogeneous plate of GFAP-, S100β- and vimentin-positive astrocytes. Using two commercial sources of primary human astrocytes as standards, we validated the expression of select astrocyte marker genes in iPSC-derived astrocytes. At functional level, iPSC-derived astrocytes were able to propagate a calcium wave after mechanical stimulation, similar to the commercially available astrocytes (FIG. 27). More importantly, human iPSC-derived astrocytes integrated into the cortex of a rodent brain after transplantation.

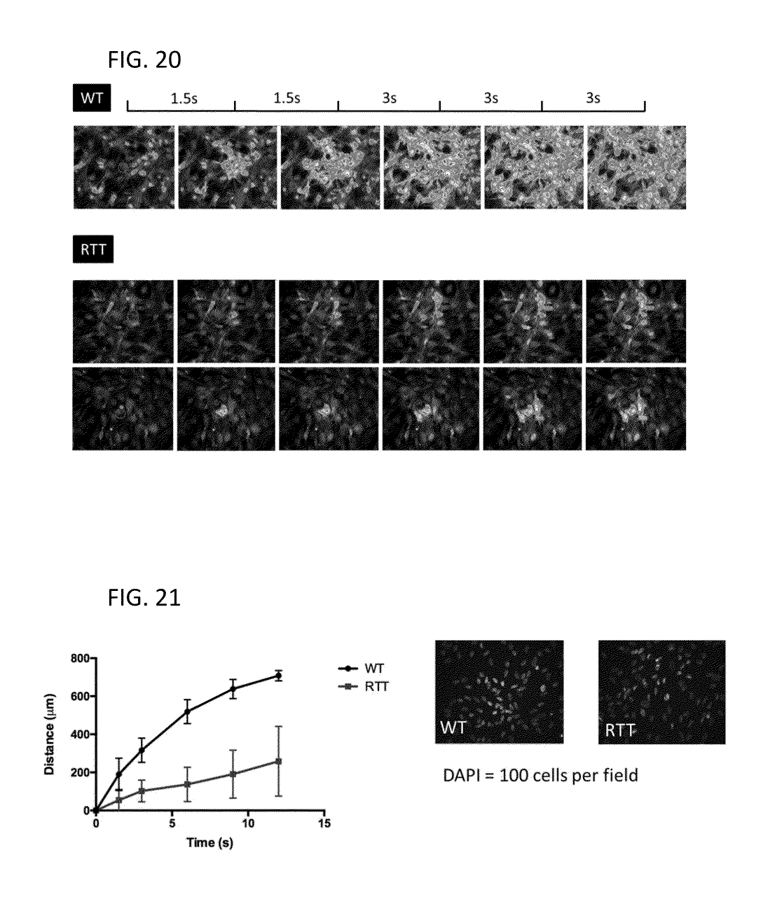

Using the protocol described above, astrocytes from both RTT and control (WT) iPSCs were derived and analyzed for target gene expression. RTT astrocytes downregulate certain secreted factors, such as bone morphogenetic proteins (BMPs). Genes involved in the glutamate pathway were also affected in RTT astrocytes (FIG. 19). Consistently, we measured the clearance of glutamate from culture media by RTT astrocytes. FIG. 19 shows that RTT astrocyte cultures accumulate glutamate in the media over time, indicating a deficiency in glutamate uptake and/or clearance by RTT cells. Finally, RTT astrocytes failed to propagate calcium waves after mechanical stimulation (FIGS. 20 and 21).

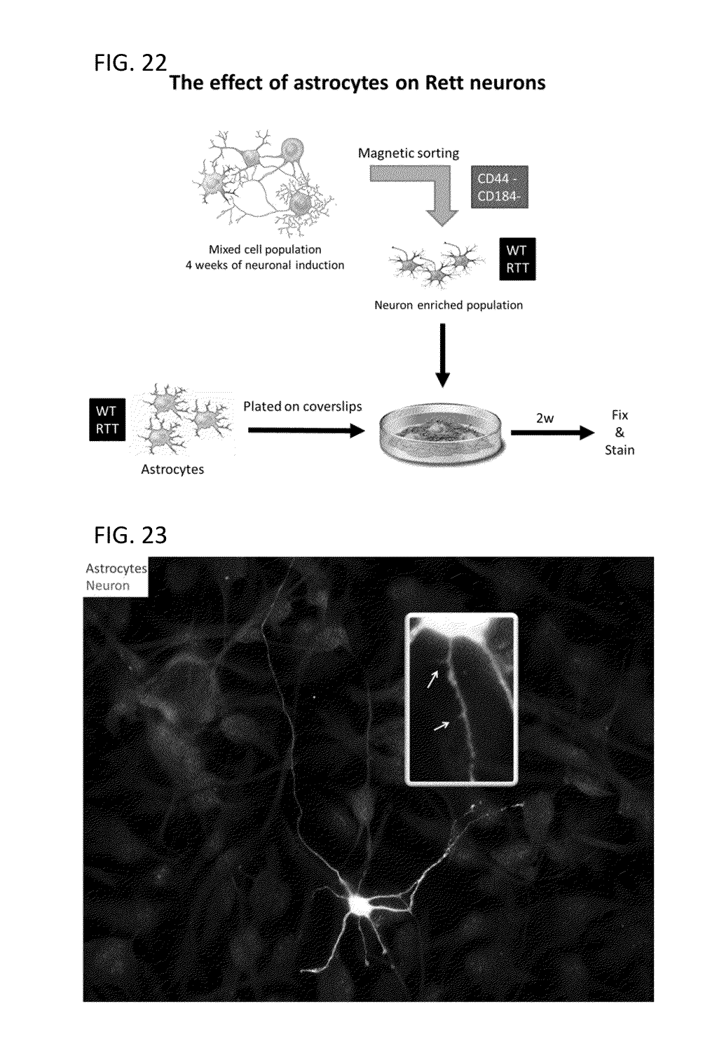

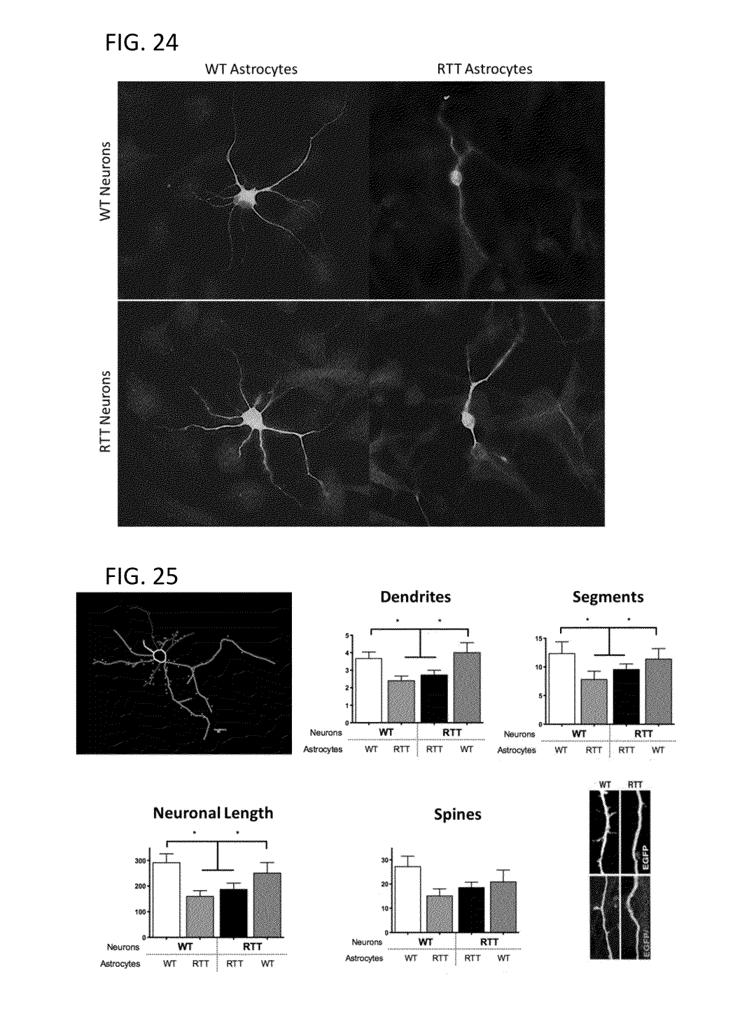

MeCP2-deficient astrocytes cause non-cell autonomous effects on neuronal dendrites and synaptic function. In MeCP2 knockout mice, the selective expression of MeCP2 in astrocytes rescues certain RTT deficits. Thus, we hypothesized that human RTT astrocytes would negatively impact co-cultured human neurons, while healthy astrocytes would promote RTT neuronal maturation. The complexity of dendritic arborization and presence of spines are important indicators of neuronal maturation. Sorted iPSC-derived neurons were plated on top of a monolayer of astrocytes. As healthy WT astrocytes create a monolayer for healthy WT neurons, neurons mature revealing complex dendritic arborization and spine formation (FIGS. 22 and 23). In contrast, RTT astrocytes negatively affect WT neurons, as can be clearly noted by morphological aspects of WT neurons. WT neurons also display immature RTT-like neuronal morphology, such as smaller soma size, bipolar, less arborized neurons with reduced number of spines (FIGS. 24 and 25). We also tested whether loss of function of MeCP2 in astrocytes was directly related to the neuronal phenotypes in our co-culture experiments. We used a lentiviral vector, carrying a shRNA against MeCP2³, to infect control astrocyte. Astrocytes derived from WT-iPSCs expressing the shMeCP2 showed a similar effect on co-cultured control neurons when compared to RTT astrocytes expressing a scramble shRNA shControl, FIG. 25. Our data strongly suggest that MeCP2 in astrocytes can strongly affect human neurons in a non-cell autonomous fashion.

We next attempted to rescue neuronal defects by culturing RTT neurons with WT astrocytes. Remarkably, RTT neurons in this environment showed a significant increase in soma size, dendrite number, and spine number, with a total neuronal length that is similar of WT neurons (FIG. 25).

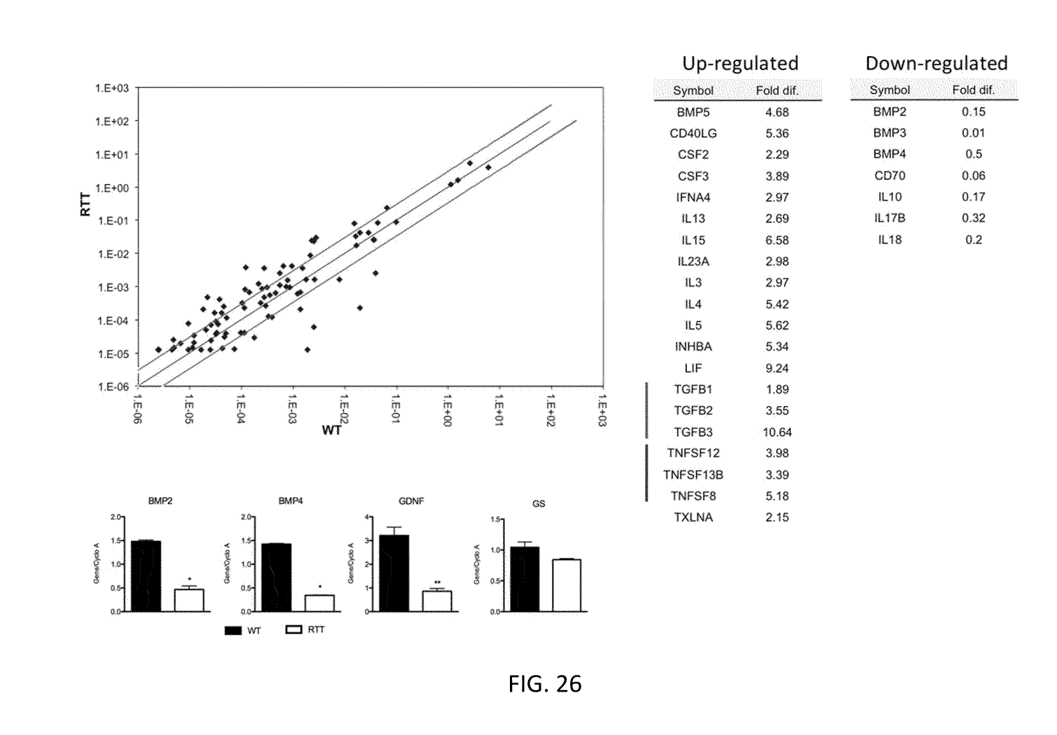

Next, we evaluated whether certain factors released from RTT astrocytes contribute to the neuronal phenotypes. Based on the initial gene expression profile and data from prior studies, we focused on dysregulation of cytokines. To determine which cytokines are affected by MeCP2 mutation, we measured cytokine expression from RTT and WT astrocytes. Among the 40 most differentially expressed cytokines, IL-8 and IL-10 showed the most dramatic differences between RTT and control WT astrocytes (FIG. 26). We validated the gene expression data using immunoassays from astrocyte supernatants. Next, we treated WT neurons with a combination of IL8 and IL10, or by mixing these two cytokines together. As a control, we treated neurons with cytokines that did not vary from RTT and WT.

### REFERENCES FOR EXAMPLE 3

1 Allaman, I., Belanger, M. & Magistretti, P. J. Astrocyte-neuron metabolic relationships: for better and for worse. Trends in neurosciences 34, 76-87, doi:10.1016/j.tins.2010.12.001 (2011).
2 Amir, R. E. et al. Rett syndrome is caused by mutations in X-linked MECP2, encoding methyl-CpG-binding protein 2. Nat Genet 23, 185-188 (1999).
3 Marchetto, M. C. et al. A model for neural development and treatment of Rett syndrome using human induced pluripotent stem cells. Cell 143, 527-539, doi:S0092-8674(10)01186-4 [pii] 10.1016/j.cell.2010.10.016 (2010).
4 Ballas, N., Lioy, D. T., Grunseich, C. & Mandel, G. Non-cell autonomous influence of MeCP2-deficient glia on neuronal dendritic morphology. Nature neuroscience 12, 311-317, doi:10.1038/nn.2275 (2009).
5 Muotri, A. R. et al. L1 retrotransposition in neurons is modulated by MeCP2. Nature 468, 443-446, doi:nature09544 [pii] 10.1038/nature09544 (2010).
6 Maezawa, I., Swanberg, S., Harvey, D., LaSalle, J. M. & Jin, L. W. Rett syndrome astrocytes are abnormal and spread MeCP2 deficiency through gap junctions. The Journal of neuroscience: the official journal of the Society for Neuroscience 29, 5051-5061, doi:10.1523/JNEUROSCI.0324-09.2009 (2009).
7 De Filippis, B. et al. Modulation of RhoGTPases improves the behavioral phenotype and reverses astrocytic deficits in a mouse model of Rett syndrome. Neuropsychopharmacology: official publication of the American College of Neuropsychopharmacology 37, 1152-1163, doi:10.1038/npp.2011.301 (2012).
8 Lioy, D. T. et al. A role for glia in the progression of Rett's syndrome. Nature 475, 497-500, doi:10.1038/nature10214 (2011).

1. A method for inhibiting a neurological disease or disorder in a subject having a deficiency or alteration in a glutamatergic pathway affecting neuron and/or glial function comprising administering an effective amount of a NMDA receptor antagonist(s) and/or modulator(s) of a glutamatergic pathway to the subject, thereby inhibiting the disease or disorder.

2. The method of claim 1, wherein the deficiency or alteration in a glutamatergic pathway affecting neuron and/or glial function is associated with TRPC6 mutation or haploid insufficiency.

3. The method of claim 2, wherein the deficiency or alteration in a glutamatergic pathway affecting neuron and/or glial function is associated with TRPC6 mutation or haploid insufficiency and MeCP2 mutation or insufficiency.

4. The method of claim 1, wherein the neurological disease or disorder is selected from a group consisting of Rett Syndrome (RTT), idiopathic autism, severe neonatal encephalopathy, schizophrenia, autism spectrum disorder (ASD) and X-linked mental retardation.

5. The method of claim 1, wherein the modulator of a glutamatergic pathway is a TRPC6 modulator(s) and/or MeCP2 modulator(s) that can modulate the phosphorylation state of CREB transcription factor to normalize neuronal or glial gene expression.

6. (canceled)

7. A method for inhibiting a neurological disease or disorder by the method of claims 1.

8.-9. (canceled)

10. The method of claim 1, wherein the NMDA receptor antagonist(s) comprises any one or a combination of 3,5-Dimethyl-tricyclo[3.3.1.13,7]decan-1-amine hydrochloride (Memantine hydrochloride), 1-Aminocyclobutane-1-carboxylic acid (ACBC), D-(−)-2-Amino-5-phosphonopentanoic acid (D-AP5), L-(+)-2-Amino-5-phosphonopentanoic acid (L-AP5), D-(−)-2-Amino-7-phosphonoheptanoic acid (D-AP7), N,N′-1,4-Butanediylbisguanidine sulfate (arcaine sulfate), (R)-4-Carboxyphenylglycine ((R)-4CPG), (S)-4-Carboxyphenylglycine ((S)-4CPG), (E)-(±)-2-Amino-4-methyl-5-phosphono-3-pentenoic acid (CGP 37849), (E)-(±)-2-Amino-4-methyl-5-phosphono-3-pentenoic acid ethyl ester (CGP 39551), [(1S)-1-[[(7-Bromo-1,2,3,4-tetrahydro-2,3-dioxo-5-quinoxalinyl)methyl]amino]ethyl]phosphonic acid hydrochloride (CGP 78608 hydrochloride), cis-4-[Phosphomethyl]-piperidine-2-carboxylic acid (CGS 19755), 7-Chloro-4-hydroxyquinoline-2-carboxylic acid (7-Chlorokynurenic acid), (2R,3S)-β-p-Chlorophenylglutamic acid ((2R,3S)-Chlorpheg), 6-Cyano-7-nitroquinoxaline-2,3-dione (CNQX), 1-[2-(4-Hydroxyphenoxy)ethyl]-4-[(4-methylphenyl)methyl]-4-piperidinol hydrochloride (Co 101244 hydrochloride; PD 174494; or Ro 63-1908), GEXXVAKMAAXLARXNIAKGCKVNCYP (Conantokin-R), GEXXYQKMLXNLRXAEVKKNA (Conantokin-T), 3-((R)-2-Carboxypiperazin-4-yl)-propyl-1-phosphonic acid ((R)-CPP), (RS)-3-(2-Carboxypiperazin-4-yl)-propyl-1-phosphonic acid ((RS)-CPP), D-4-[(2E)-3-Phosphono-2-propenyl]-2-piperazinecarboxylic acid (D-CPP-ene; Midafotel; or SDZ EAA 494), (9α,13α,14α)-3-Methoxy-17-methylmorphinan hydrobromide (Dextromethorphan hydrobromide), 5,7-Dichloro-4-hydroxyquinoline-2-carboxylic acid (5,7-Dichlorokynurenic acid), (±)-1-(1,2-Diphenylethyl)piperidine maleate, α-(4-Chlorophenyl)-4-[(4-fluorophenyl)methyl]-1-piperidineethanol (Eliprodil), 2-Phenyl-1,3-propanedioldicarbamate (Felbamate), N-[2-Amino-6-[[4-fluorophenyl)methyl]amino]-3-pyridinyl]carbamic acid ethyl ester maleate (Flupirtine maleate), 4,6-Dichloro-3-[(1E)-3-oxo-3-(phenylamino)-1-propenyl]-1H-indole-2-carboxylic acid sodium salt (Gavestinel; GV 150526A), (S)-(−)-3-Amino-1-hydroxypyrrolidin-2-one ((S)-(−)-HA-966), (R)-(+)-3-Amino-1-hydroxypyrrolidin-2-one ((R)-(+)-HA-966), (6aS,10aS)-3-(1,1-Dimethylheptyl)-6a,7,10,10a-tetrahydro-1-hydroxy-6,6-dimethyl-6H-dibenzo[b,d]pyran-9-methanol (HU 211; or Dexanabinol), N—(N-(4-Hydroxyphenylacetyl)-3-aminopropyl)-(N′-3-aminopropyl)-1,4-butanediamine (N-(4-Hydroxyphenylacetyl)spermine), (1R*,2S*)-erythro-2-(4-Benzylpiperidino)-1-(4-hydroxyphenyl)-1-propanol hemitartrate (Ifenprodil hemitartrate), (1S*,2S*)-threo-2-(4-Benzylpiperidino)-1-(4-hydroxyphenyl)-1-propanol hemitartrate (threo Ifenprodil hemitartrate), 2-(2-Chlorophenyl)-2-(methylamino)cyclohexanone hydrochloride (Ketamine hydrochloride), (S)-(+)-2-(2-Chlorophenyl)-2-(methylamino)cyclohexanone hydrochloride ((S)-(+)-Ketamine hydrochloride), trans-2-Carboxy-5,7-dichloro-4-phenylaminocarbonylamino-1,2,3,4-tetrahydroquinoline (L-689,560), 7-Chloro-3-(cyclopropylcarbonyl)-4-hydroxy-2(1H)-quinolinone (L-701,252), 7-Chloro-4-hydroxy-3-(3-phenoxy)phenyl-2(1H)-quinolinone (L-701,324), 4-(4-Chlorophenyl)-4-hydroxy-N,N-dimethyl-α,α-diphenyl-1-piperidinebutanamide hydrochloride (Loperamide hydrochloride), (2R*,4S*)-4-(1H-Tetrazol-5-ylmethyl)-2-piperidinecarboxylic acid (LY 233053), [3 S-(3α,4aα,6β,8aα)]-Decahydro-6-(phosphonomethyl)-3-isoquinolinecarboxylic acid (LY 235959), (5R,10S)-(+5-Methyl-10,11-dihydro-5H-dibenzo[a,d]cylcohepten-5,10-imine maleate ((−)-MK 801 maleate), (5 S,10R)-(+)-5-Methyl-10,11-dihydro-5H-dibenzo[a,d]cyclohepten-5,10-imine maleate ((+)-MK 801 maleate; MK-801; or Dizocilpine), 2-Amino-2-(2-chlorophenyl)cyclohexanone hydrochloride (Norketamine hydrochloride), D-[[1-(2-Nitrophenyl)ethyl]carbamoyl]-2-amino]-5-phosphonopentanoic acid (NPEC-caged-D-AP5), 4,4′-(Pentamethylenedioxy)dibenzamidine bis-2-hydroxyethanesulfonate salt (Pentamidine isethionate), 1-(1-Phenylcyclohexyl)piperidine hydrochloride (Phencyclidine hydrochloride), 4-(Phosphonomethyl)-2-piperazinecarboxylic acid (PMPA), (2S*,3R*)-1-(Phenanthren-2-carbonyl)piperazine-2,3-dicarboxylic acid (PPDA), (2R*,4S*)-4-(3-Phosphonopropyl)-2-piperidinecarboxylic acid (PPPA; or LY 257883), 2-Amino-N-(1-methyl-1,2-diphenylethyl)acetamide hydrochloride (Remacemide hydrochloride; or FPL 12924AA), 1-[2-(4-Chlorophenyl)ethyl]-1,2,3,4-tetrahydro-6-methoxy-2-methyl-7-isoquinolinol hydrochloride (Ro 04-5595 hydrochloride), (αR,βS)-α-(4-Hydroxyphenyl)-β-methyl-4-(phenylmethyl)-1-piperidinepropanol maleate (Ro 25-6981 maleate), 3,4-Dimethoxy-N-[4-(3-nitrophenyl)-2-thiazolyl]benzenesulfonamide (Ro 61-8048), 4-[3-[4-(4-Fluorophenyl)-1,2,3,6-tetrahydro-1(2H)-pyridinyl]-2-hydroxypropoxy]benzamide hydrochloride (Ro 8-4304 hydrochloride), (S)-α-Amino-2′,4′-dichloro-4-hydroxy-5-(phosphonomethyl)[1,1′-biphenyl]-3-propanoic acid (SDZ 220-040), (S)-α-Amino-2′-chloro-5-(phosphonomethyl)[1,1′-biphenyl]-3-propanoic acid (SDZ 220-581), N,N′-1,10-Decanediylbisguanidine sulfate (Synthalin sulfate), 3-Chloro-4-fluoro-N-[4-[[2-(phenylcarbonyl)hydrazino]carbonyl]benzyl]benzenesulfonamide (TCN 201), N-(Cyclohexylmethyl)-2-[(5-[(phenylmethyl)amino]-1,3,4-thiadiazol-2-yl}thio]acetamide (TCN 213), 1,3-Dihydro-5-[3-[4-(phenylmethyl)-1-2H-benzimidazol-2-one (TCS 46b), nitrous oxide (N₂O), Dextrorphan, Selfotel, Amantadine, Dextrallorphan, Eticyclidine, Gacyclidine, Ibogaine, Ethanol, Methoxetamine, Rolicyclidine, Tenocyclidine, Methoxydine (4-meo-pcp), Tiletamine, Xenon, Neramexane, Etoxadrol, Dexoxadrol, NEFA, Delucemine, 8A-PDHQ, Aptiganel (Cerestat; or CNS-1102), HU-211, Rhynchophylline, 1-Aminocyclopropanecarboxylic acid, 7-Chlorokynurenate, Kynurenic acid, and/or Lacosamide, and/or derivatives thereof.

11. (canceled)

12. The method of claim 1, wherein the modulator of a glutamatergic pathway is any one or more of Acetazolamide (N-(5-sulfamoyl-1,3,4-thiadiazol-2-yl)acetamide)), BIX-01294 (having the formula C₂₈H₃₈N₆O₂.3HCl (CAS No. 1392399-03-9, 935693-62-2), a G9a histone methyltransferease (G9aHMTase) inhibitor, Zonisamide (benzo[d]isoxazol-3-ylmethanesulfonamide), a sulfonamide anticonvulsant, Forskolin ((3R,4aR,5S,6S,6aS,10S,10aR,10bS)-6,10,10b-trihydroxy-3,4a,7,7,10a-pentamethyl-1-oxo-3-vinyldodecahydro-1H-benzo[f]chromen-5-yl acetate), a labdane diterpene, an adenylyl cyclase activator, Tubastatin A (N-Hydroxy-4-(2-methyl-1,2,3,4-tetrahydro-pyrido[4,3-b]indol-5-ylmethyl)-benzamide), 7,8 Dihydroxyflavone, Topiramate (2,3:4,5-Bis-O-(1-methylethylidene)-beta-D-fructopyranose sulfamate), a histone deacetylase HDAC6 inhibitor, AR-A014418 (N-[(4-Methoxyphenyl)methyl]-N′-(5-nitro-2-thiazolyl)urea), a glycogen synthase kinase 3 (GSK3) inhibitor, Amitriptyline (3-(10,11-dihydro-5H-dibenzo[a,d]cycloheptene-5-ylidene)-N,N-dimethylpropan-1-amine), a serotonin-norepinephrine reuptake inhibitor, a 5-HT_{2A}, 5-HT_{2C}, 5-HT₃, 5-HT₆, 5-HT₇, α1-adrenergic, H₁, H₂, H₄, and mACh receptor antagonist, σ1 receptor agonist, a sodium, calcium, and potassium channel blocker, a TrkA and TrkB receptor agonist, LM22A-3, a BDNF mimic, NBI-31772, an insulin-like growth factor 1 (IGF-1) potentiator, ING-135, an ampakine, an α-amino-3-hydroxy-5-methyl-4-isoxazolepropionic acid (AMPA) receptor potentiator, CX-546, LM22A-4, Aripiprazole (7-[4-[4-(2,3-Dichlorophenyl)piperazin-1-yl]butoxy]-3,4-dihydroquinolin-2(1H)-one), a dopamine and serotonin receptor modulator, Hyperzine A or huperzine A ((1R,9S,13E)-1-Amino-13-ethylidene-11-methyl-6-azatricyclo[7.3.1.0^{2,7}]trideca-2(7),3,10-trien-5-one), an acetylcholinesterase and NMDA inhibitor, MK-677 ((R)-1′-(2-methylalanyl-O-benzyl-D-seryl)-1-(methylsulfonyl)-1,2-dihydrospiro[indole-3,4′-piperidine]), a growth hormone secretagogue, Chlormezanone (2-(4-chlorophenyl)-3-methyl-1,1-dioxo-1,3-thiazinan-4-one), a GABA receptor potentiator, Cyctothiazide (3-(bicyclo[2.2.1]hept-5-en-2-yl)-6-chloro-3,4-dihydro-2H-1,2,4-benzothiadiazine-7-sulfonamide 1,1-dioxide), a positive modulator of AMPA receptor, Pioglitazone ((RS)-5-(4-[2-(5-ethylpyridin-2-yl)ethoxy]benzyl)thiazolidine-2,4-dione), an agonist of the peroxisome proliferator activated receptor gamma (PPARγ), Memantine (3,5-dimethyltricyclo[3.3.1.1^{3,7}]decan-lamine or 3,5-dimethyladamantan-1-amine), an NMDA receptor antagonist, a glutamate antagonist, a medium, plasma or extracellular fluid deficient in glutamine or glutamate, a glutamate antagonist, DON (6-Diazo-5-oxo-L-norleucine or (Z,5S)-5-Amino-1-diazonio-6-hydroxy-6-oxohex-1-en-2-olate; chemical formula C₆H₉N₃O₃ (CAS No. 157-03-9)), a glutamine antagonist, CBX (carbenoxolone, (3P)-3-[(3-carboxypropanoyl)oxy]-11-oxoolean-12-en-30-oic acid, or (2S,4aS,6aS,6bR,8aR,10S,12aS,12bR,14bR)-10-(3-carboxypropanoyloxy)-2,4a,6a,6b,9,9,12a-heptamethyl-13-oxo-1,2,3,4,4a,5,6,6a,6b,7,8,8a,9,10,11,12,12a,12b,13,14b-icosahydropicene-2-carboxylic acid); chemical formula C₃₄H₅₀O₇ (CAS No. 5697-56-3)), a gap-junction blocker, Valproic Acid (VPA), a histone deacetylase (HDAC) inhibitor, DAPT (LY-374973, N—[N-(3,5-Difluorophenacetyl)-L-alanyl]-S-phenylglycine t-butyl ester), a gamma-secretase inhibitor, Aminoguanidine, an iNOS inhibitor, Dizocilpine (INN) (MK-801 or [5R,10S]-[+]-5-methyl-10,11-dihydro-5H-dibenzo[a,d]cyclohepten-5,10-imine), a NMDA receptor antagonist, Curcumin ((1E,6E)-1,7-Bis(4-hydroxy-3-methoxyphenyl)-1,6-heptadiene-3,5-dione), Resveratrol (3,5,4′-trihydroxy-trans-stilbene), a curcuminoid, a natural phenol, an anti-inflammatory agent, Ceftriaxone ((6R,7R)-7-{[(2Z)-2-(2-amino-1,3-thiazol-4-yl)->2-(methoxyimino)acetyl]amino}-3-{[(2-methyl-5,6-dioxo-1,2,5,6-tetrahydro-1,2,4-triazin-3-yl)thio]methyl}-8-oxo-5-thia-1-azabicyclo[4.2.0]oct-2-ene-2-carboxylic acid), Epigallocatechin (Gallocatechol or gallocatechin), Gingerol ((S)-5-hydroxy-1-(4-hydroxy-3-methoxyphenyl)-3-decanone), Gly-Pro-Glu, or ‘Insulin-like growth factor 1 (IGF-1).

13. The method of claim 7, wherein administration of cytokine(s) and/or modulator(s) of cytokine activity restores synaptic deficiency, increases dendritic spine density, increases glutamatergic synapses, restores neurite soma size, restores neurite length, restores number of glutamate vesicles, restores VGLUT1 puncta or cluster along MAP2-positive processes of neurons, restores dendritic complexity measured as a function of number of crossings for each distance from the cell body, restores neuronal nuclei size, restores neuronal nuclei sphericity, restores neuronal spike frequency, increases transient Ca²⁺ concentration, increases repetitive intracellular Ca²⁺ concentration, increases amplitude of Ca²⁺ oscillation, restores Na⁺ current density, restores action potential, increases action potential burst trains, restores firing rate of neurons in whole cell patch clamp recording, restores synapsin puncta or cluster along MAP2-positive processes of neurons, restores PSD-95 expression level, restores neuronal networks, restores astrocyte networks, restores calcium signaling, restores calcium wave propagation to surrounding cells upon mechanical stimulation of an individual cell, and/or normalizes gene expression in the neuronal or glial cell within the central nervous system or peripheral nervous system of a patient or a combination thereof.

14. (canceled)

15. The method of claim 7, wherein the modulator(s) of cytokine activity is an agent which decreases production, decreases secretion, decreases half-life, decreases activity or neutralizes activity of any one or more of Bone morphogenetic protein 5 (BMP5), CD40 Ligand, colony stimulating factor 2 (CSF2 or Granulocyte macrophage colony-stimulating factor 2), CSF-3, interferon A4 (IFNA4), interleukin 13 (IL-13), IL-15, IL-23A, IL-3, IL-4, IL-5, Inhibin, beta A, (INHBA), Leukemia inhibitory factor (LIF), tumor growth factor beta-1 (TGF-B1), tumor growth factor beta-2 (TGF-β2), tumor growth factor beta-3 (TGF-β3), Tumor necrosis factor superfamily 12 (TNF-SF12), Tumor necrosis factor superfamily 13 (TNF-SF13B), Tumor necrosis factor superfamily 8 (TNF-SF8), or Alpha-taxilin (TXLNA).

16. (canceled)

17. The method of claim 15, wherein the agent is a nucleic acid, protein or synthetic chemical compound and/or derivatives thereof.

18. The method of claim 17, wherein the nucleic acid comprises a gene therapy vector, and a coding sequence or part of a coding sequence for any one or more of Bone morphogenetic protein 5 (BMP5), CD40 Ligand, colony stimulating factor 2 (CSF2 or Granulocyte macrophage colony-stimulating factor 2), CSF-3, interferon A4 (IFNA4), interleukin 13 (IL-13), IL-15, IL-23A, IL-3, IL-4, IL-5, Inhibin, beta A, (INHBA), Leukemia inhibitory factor (LIF), tumor growth factor beta-1 (TGF-β1), tumor growth factor beta-2 (TGF-β2), tumor growth factor beta-3 (TGF-β3), Tumor necrosis factor superfamily 12 (TNF-SF12), Tumor necrosis factor superfamily 13 (TNF-SF13B), Tumor necrosis factor superfamily 8 (TNF-SF8), Alpha-taxilin (TXLNA), BMP2, BMP3, BMP4, CD70, IL-10, IL-17B, or IL-18.

19.-25. (canceled)

26. A method for screening candidate drugs that inhibit a neurological disease associated with a MeCP2 mutation, haploid insufficiency or a X-linked gene mutation or aberrant activity comprising:
a. Inducing iPSC from a male subject to undergo neuronal differentiation;
b. Contacting the neuronal differentiated iPSC-derived cells or neurons with candidate drugs; and
c. Analyzing the treated cells in (b) for an increase in neuronal networks, dendritic spine density, synapses, soma size, neuronal excitation, or calcium signaling, wherein such increase is indicative of inhibiting the neurological disease when compared to mock treated cells or when compared to treated differentiated iPSC-derived cells or neurons from a wild-type or unaffected male subject.

27. The method of claim 26, wherein the iPSC from (a) exhibits reduced variability associated with dosage compensation of the X-chromosome in mammals which results in the differentiating or differentiated cell derived from an induced pluripotent stem cell (iPSC) of female origin either expressing genes from the maternal or paternal X-chromosome.

28.-38. (canceled)

39. A method for diagnosing whether a subject has an increased risk for developing Rett Syndrome (RTT), idiopathic autism, severe neonatal encephalopathy, schizophrenia, X-linked mental retardation, deficiency in glutamatergic pathways of the glial cells, neuronal networks with a deficiency in glutamatergic pathways affecting the formation of excitatory synapses, and/or a subset of neurological disorders with a deficiency in glutamatergic pathways affecting the formation of excitatory synapses, comprising detecting in the cells from the subject a mutation in a TRPC6 or MeCP2 gene and determining whether the cell exhibit decreased neuronal gene expression affecting one or more pathways comprising neurotrophin signaling pathway, IGF signaling pathway, pathway with synaptic protein, NeuN gene pathway, and glutamate-glutamine transport pathway.

40. The method of claim 39, wherein decreased neuronal gene expression affecting the neurotrophin signaling pathway comprises BDNF, NGFR, or NTF4.

41. The method of claim 39, wherein decreased neural gene expression affecting the IGF signaling pathway comprises IGF1 or IGF2.

42. The method of claim 39, wherein decreased neuronal gene expression affecting the pathway with synaptic protein comprises PSD-95, VGlut1, VGlut2, or syn1.
